(19) United States
(12) Patent Application Publication
(10) Pub. No.: US 2023/0270086 A1
(43) Pub. Date: Aug. 31, 2023

(54) TRANSGENIC ANIMALS EXPRESSING HEAVY CHAIN ANTIBODIES

(71) Applicant: KISOJI BIOTECHNOLOGY INC., Montréal (CA)

(72) Inventors: Wenyang HOU, Montréal (CA); Xin LI, Montréal (CA); Luis DA CRUZ, Montréal (CA); Ashwani GUPTA, Montréal (CA); David S. YOUNG, Montréal (CA)

(21) Appl. No.: 18/015,304

(22) PCT Filed: Jul. 12, 2021
(86) PCT No.: PCT/CA2021/050951

§ 371 Date: Jan. 9, 2023

Related U.S. Application Data
(60) Provisional application No. 63/076,096, filed on Sep. 9, 2020.
(60) Provisional application No. 63/051,180, filed on Jul. 13, 2020.

(51) Int. Cl. A01K 67/027; C07K 16/00

(52) U.S. Cl. CPC A01K 67/0275; C07K 16/00; A01K 2227/105; A01K 2217/05; A01K 2267/01; C07K 2317/569; C07K 2317/522; C07K 2317/22

(57) ABSTRACT

The present disclosure generally relates to transgenic animals comprising germline modifications at an immunoglobulin heavy chain (IgH) locus for expressing heavy chain antibodies (HCAbs) as well as nucleic acid constructs, cells and methods for generating same. The present disclosure also relates to binding agents comprising sequences derived from the heavy chain antibodies produced by the transgenic animals.

### TECHNICAL FIELD

The present disclosure generally relates to transgenic animals comprising germline modifications at an immunoglobulin heavy chain (IgH) locus for expressing heavy chain antibodies (HCAbs) as well as nucleic acid constructs, cells and methods for generating same. The present disclosure also relates to binding agents comprising sequences derived from the heavy chain antibodies produced by the transgenic animals.

### BACKGROUND

Camelids and cartilaginous fishes naturally produce antibodies composed of functional homodimeric heavy chain antibodies (HCAbs) (Hamers-Casterman et al., 1993; Muyldermans and Smider, 2016). The heavy chains of HCAbs lack the first constant domain (CH1) and differs from classical antibodies by only a few amino acids substitutions normally involved in light chain pairing (Muyldermans et al., 1994; Vu et al., 1997). These substitutions (Val37Phe/Tyr, Gly44Glu, Leu45Arg, and Trp47Gly) are present in framework region 2 (FR2). The antigen-binding domain of HCAbs is referred to as single domain antibody (sdAb), VHH or Nanobody®. sdAbs have a molecular weight of around 15 kDa which makes them amenable to applications that require enhanced tissue penetration or rapid clearance, such as radioisotope-based imaging.

The variable region of camelid HCAbs have longer CDRH1 and CDRH3 loops compared with the respective classical CDRs, increasing the paratope size. The longer CDRs bind epitopes, which are more concave than those of classical antibodies. They can also inhibit enzymes by entering clefts in catalytic sites (Sircar et al., The Journal of Immunology, 186, 2011).

Single domain antibodies are currently exploited as therapeutics and diagnostics in various antibody-like formats including multi-specific and multivalent formats.

As antibodies containing camelid sequences are expected to induce an immune response in humans, recent efforts were mainly focused at developing human single domain antibodies in transgenic mice. These mouse models were designed by inactivating or replacing portions of the mouse IgH locus to encode human variable (V), diversity (D), and joining (J) segments (see WO2016/062990A1, US2011/0145937A1).

Although, single domain antibodies can be obtained by immunization of camelids, this approach is time-consuming and expensive. Moreover, large amount of immunogen is required, and the repertoire of antibody obtained is limited.

Transgenic mice expressing single domain antibodies comprising camelid VHHs, camelid/human hybrid VHs or human VHs have been generated by random integration of rearranged or unrearranged minilocus into the genome of IgM-deficient mice (Zhou et al. J. Immunol., 175(6):3369-79 (2005); Janssens et al., PNAS 103(41):15130-15135 (2006); Drabek et al. Front. Immunol. 7:619 (2016), U.S. Pat. No. 8,502,014). These models suffer the limitation of having low expression and offering poor diversity in the pool of HCAbs generated by immunization.

### SUMMARY

The Applicant provides herein, among other things, transgenic non-human animals that comprise germline modifications at an immunoglobulin heavy chain (IgH) locus for producing camelid single domain antibodies.

Genetically modified animals are provided herein to facilitate the production of camelid single domain antibodies.

In some aspects of the disclosure, transgenic animals are used for expression of HCAbs of various isotype and of diverse genetic backgrounds.

In other aspect of the disclosure, transgenic animals are provided for increasing the diversity in the HCAb repertoire generated.

The pool of antigen-specific heavy chain only antibodies (HCAbs) generated upon immunization of the transgenic animals disclosed herein is analyzed and HCAb candidates are selected.

In some embodiments, the HCAbs are used in the making of a binding agent.

In some embodiments, the HCAbs are used in the making of a therapeutic.

In some embodiments, the HCAbs are used in the making of a diagnostic.

In some aspects and embodiments, the disclosure relates to a transgenic non-human animal that comprises germline modifications at an immunoglobulin heavy chain (IgH) locus.

In some embodiments, all modifications are on the same allele. In other embodiments both alleles may be the same. Yet in other embodiments, both alleles may be different.

In some embodiments, the transgenic non-human animal of the present disclosure comprises a germline modification selected, for example from the group consisting of:
a. deletion of the CH1 domain of an endogenous non-human animal gamma globulin gene, or;
b. deletion of the CH1 domain of at least one endogenous non-human animal gamma globulin in combination with a complete or partial deletion of at least one other endogenous non-human animal gamma globulin gene.

In other embodiments, the transgenic non-human animal of the present disclosure comprises a germline modification selected, for example from the group consisting of:
a. modification of the CH1 domain of an endogenous non-human animal gamma globulin gene, or;
b. modification of the CH1 domain of at least one endogenous non-human animal gamma globulin in combination with a complete or partial deletion of at least one other endogenous non-human animal gamma globulin gene.

In some embodiments, the modification of the CH1 domain results in a non-functional CH1 domain. In some embodiments, the non-functional CH1 domain modification is not able to pair with a light chain.

In some embodiments, the transgenic non-human animal of the present disclosure is a mouse comprising a germline modification selected, for example from the group consisting of:
a. deletion of the CH1 domain of an endogenous mouse γ3 gene, γ1 gene, γ2b gene and/or or γ2a gene, or;
b. deletion of the CH1 domain of at least one endogenous mouse gene selected from γ3 gene, γ1 gene, γ2b gene and/or or γ2a gene in combination with a complete or partial deletion of at least one endogenous mouse gene selected from γ3 gene, γ1 gene, γ2b gene and/or or γ2a gene.

In other embodiments, the transgenic non-human animal of the present disclosure is a mouse comprising a germline modification selected, for example from the group consisting of:
a. modification of the CH1 domain of an endogenous mouse γ3 gene, γ1 gene, γ2b gene and/or or γ2a gene, or;
b. modification of the CH1 domain of at least one endogenous mouse gene selected from γ3 gene, γ1 gene, γ2b gene and/or or γ2a gene in combination with a complete or partial deletion of at least one endogenous mouse gene selected from γ3 gene, yl gene, γ2b gene and/or or γ2a gene.

In an exemplary embodiment, the germline modification comprises deletion of the CH1 domain of an endogenous γ3 gene.

In an exemplary embodiment, the germline modification comprises deletion of the CH1 domain of an endogenous γ1 gene.

In another exemplary embodiment, the germline modification comprises deletion of the CH1 domain of an endogenous γ2b gene.

In another exemplary embodiment, the germline modification comprises deletion of the CH1 domain of an endogenous γ2a gene.

In another exemplary embodiment, the germline modification comprises deletion of the CH1 domain of an endogenous γ3 gene, γ1 gene, γ2b gene and γ2a gene.

In a further exemplary embodiment, the germline modification comprises deletion of the CH1 domain of an endogenous γ2a gene and deletion of an endogenous γ2b gene.

In another exemplary embodiment, the germline modification comprises deletion of the CH1 domain of an endogenous γ3 gene and γ2a gene and deletion of the γ2b gene.

In some embodiments the transgenic non-human animal is a transgenic mouse comprising endogenous mouse V, D and J segments and at least one endogenous mouse IgG constant region gene lacking a functional CH1 domain.

In some embodiments, the transgenic mouse is capable of expressing heavy chain only antibodies (HCAbs).

In some embodiments, the transgenic mouse does not comprise foreign V, D or J segments.

In some embodiments, the transgenic mouse does not comprise camelid V, D or J segments.

In some embodiments, the transgenic mouse comprises camelid V, D and/or J segments.

In some embodiments the transgenic non-human animal is a transgenic mouse capable of expressing heavy chain only antibodies (HCAbs) comprising a mouse VH polypeptide comprising camelid canonical framework mutations at position 37, 44, 45 and/or 47.

In some embodiments, the transgenic non-human animal has an IgH locus comprising unrearranged variable (V), diversity (D) and/or joining (J) gene segments from a mammal.

In some embodiment, the unrearranged camelid V, D and/or J gene segments include associated introns comprising recombination signal sequences (RSS) for VDJ rearrangement.

In some embodiment, the unrearranged camelid V segments include surrounding regulatory regions, intronic sequences, leader sequences and RSS.

In some embodiment, the unrearranged camelid D segments include surrounding camelid regulatory regions, camelid intronic sequences, camelid leader sequences and camelid RSS.

In some embodiment, the unrearranged camelid J segments include surrounding camelid regulatory regions, camelid intronic sequences, camelid leader sequences and camelid RSS.

In some embodiments, the V, D and/or J gene segments are from more than one mammal species.

In some embodiments, the mammal is a camelid. In some embodiments, the mammal is a human. In some embodiments, the mammal is a rodent. In some embodiments, the mammal is a non-human mammal.

In some embodiments, the IgH locus comprises endogenous V gene segments of the transgenic non-human animal. In other embodiments, all V gene segments are endogenous.

In some embodiments, the IgH locus comprises V gene segments that are foreign to the transgenic non-human animal.

In some embodiments, the foreign V gene segment(s) is(are) inserted into the transgenic non-human animal genome (e.g., at an IgH locus). In other embodiments, the foreign V gene segment(s) replace(s) one or more endogenous V gene segments of the transgenic non-human animal. Accordingly, in some embodiments, the transgenic non-human animal comprises endogenous V gene segments, foreign V gene segments and combination of endogenous and foreign V gene segments. In other embodiments, the foreign V gene segment(s) replace(s) all endogenous V gene segments of the transgenic non-human animal.

In some embodiments, the IgH locus comprises endogenous D gene segments of the transgenic non-human animal. In other embodiments, all D gene segments are endogenous.

In some embodiments, the IgH locus comprises D gene segments that are foreign to the transgenic non-human animal.

In some embodiments, the foreign D gene segment(s) is(are) inserted into the transgenic non-human animal genome (e.g., at an IgH locus). In other embodiments, the foreign D gene segment(s) replace(s) one or more endogenous D gene segments of the transgenic non-human animal. Accordingly, in some embodiments, the transgenic non-human animal comprises endogenous D gene segments, foreign D gene segments and combination of endogenous and foreign D gene segments. In other embodiments, the foreign D gene segment(s) replace(s) all endogenous D gene segments of the transgenic non-human animal.

In some embodiments, the IgH locus comprises endogenous J gene segments of the transgenic non-human animal. In other embodiments, all J gene segments are endogenous.

In some embodiments, the IgH locus comprises J gene segments that are foreign to the transgenic non-human animal.

In some embodiments, the foreign J gene segment(s) is(are) inserted into the transgenic non-human animal genome (e.g., at an IgH locus). In other embodiments, the foreign J gene segment(s) replace(s) one or more endogenous J gene segments of the transgenic non-human animal. Accordingly, in some embodiments, the transgenic non-human animal comprises endogenous J gene segments, foreign J gene segments and combination of endogenous and foreign J gene segments. In other embodiments, the foreign J gene segment(s) replace(s) all endogenous J gene segments of the transgenic non-human animal.

In some embodiments, the replacement or insertion of V, D and/or J gene segments occurs at a site where the natural V, D and/or J gene segments are respectively located.

In some embodiments, the transgenic non-human animal comprises variable (V), diversity (D) and/or joining (J) gene segments from a camelid or from another mammal.

For example, the V, D and/or J segments may be from a camelid, from a human, from a rodent or from a combination thereof.

Transgenic non-human animals carrying CH1 deletions disclosed herein may be modified to comprise camelid V, D and/or J gene segments. Alternatively, transgenic non-human animals carrying CH1 deletions disclosed herein may be modified to comprise human V, D and/or J segments. Moreover, transgenic non-human animals carrying CH1 deletions disclosed herein may be modified to comprise a combination of camelid and human V, D and/or J segments. Also, the transgenic non-human animals carrying CH1 deletions disclosed herein may be modified to comprise a combination of camelid and mouse V, D and/or J segments. In addition, the transgenic non-human animals carrying CH1 deletions disclosed herein may be modified to comprise a combination of human and mouse V, D and/or J gene segments.

Camelid V, D and/or J gene segments are particularly contemplated.

In some embodiments the camelid V, D and/or J segments are unrearranged.

In some embodiment, the unrearranged camelid V, D and/or J gene segments includes associated introns comprising recombination signal sequences for VDJ rearrangement.

In some embodiment, the unrearranged camelid V segments include surrounding regulatory regions, intronic sequences, leader sequences and RSS.

In some embodiment, the unrearranged camelid D segments include surrounding camelid regulatory regions, camelid intronic sequences, camelid leader sequences and camelid RSS.

In some embodiment, the unrearranged camelid J segments include surrounding camelid regulatory regions, camelid intronic sequences, camelid leader sequences and camelid RSS.

In some embodiments, the camelid is from the Lama genus.

In some embodiments, the camelid is from the Vicugna genus.

In some embodiments, the camelid is from the Camelus genus.

In some embodiments the camelid is from the species Lama glama. In some embodiments the camelid is from the species Vicugna pacos. In some embodiments the camelid is from the species Vicugna vicunia. In some embodiments the camelid is from the species Lama guanicoe. In some embodiments the camelid is from the species Camelus Bactrianus. In some embodiments the camelid is from the species Camelus Dromedarius.

In some embodiments, camelid V gene segments are inserted within the animal genome in such a manner that some or all endogenous V gene segments are preserved. In exemplary embodiments, all endogenous V gene segments are preserved. In exemplary embodiments, at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90 endogenous V segments are removed or replaced. Accordingly, in some embodiments, at least 1 endogenous V segment is removed or replaced. In some embodiments, at least 2 endogenous V segments are removed or replaced. In some embodiments, at least 3 endogenous V segments are removed or replaced. In some embodiments, at least 4 endogenous V segments are removed or replaced. In some embodiments, at least 5 endogenous V segments are removed or replaced. In some embodiments, at least 6 endogenous V segments are removed or replaced. In some embodiments, at least 7 endogenous V segments are removed or replaced. In some embodiments, at least 8 endogenous V segments are removed or replaced. In some embodiments, at least 9 endogenous V segments are removed or replaced. In some embodiments, at least 10 endogenous V segments are removed or replaced. In some embodiments, at least 15 endogenous V segments are removed or replaced. In some embodiments, at least 20 endogenous V segments are removed or replaced. In some embodiments, at least 25 endogenous V segments are removed or replaced. In some embodiments, at least 30 endogenous V segments are removed or replaced. In some embodiments, at least 40 endogenous V segments are removed or replaced. In some embodiments, at least 50 endogenous V segments are removed or replaced. In some embodiments, at least 60 endogenous V segments are removed or replaced. In some embodiments, at least 70 endogenous V segments are removed or replaced. In some embodiments, at least 80 endogenous V segments are removed or replaced. In some embodiments, at least 90 endogenous V segments are removed or replaced. In some embodiments, at least 100 endogenous V segments are removed or replaced. In some embodiments, more than 100 endogenous V segments are removed or replaced. In exemplary embodiments, all endogenous V segments are removed or replaced.

In some embodiments, camelid D gene segments are inserted within the animal genome in such a manner that some or all endogenous D gene segments are preserved. In exemplary embodiments, all endogenous D segments are preserved. In exemplary embodiments, at least 1 endogenous D segment is removed or replaced. In some embodiments, at least 2 endogenous D segments are removed or replaced. In some embodiments, at least 3 endogenous D segments are removed or replaced. In some embodiments, at least 4 endogenous D segments are removed or replaced. In some embodiments, at least 5 endogenous D segments are removed or replaced. In some embodiments, at least 6 endogenous D segments are removed or replaced. In some embodiments, at least 7 endogenous D segments are removed or replaced. In some embodiments, at least 8 endogenous D segments are removed or replaced. In some embodiments, at least 9 endogenous D segments are removed or replaced. In some embodiments, at least 10 endogenous D segments are removed or replaced. In some embodiments, at least 11 endogenous D segments are removed or replaced. In some embodiments, at least 12 endogenous D segments are removed or replaced. In some embodiments, at least 13 endogenous D segments are removed or replaced. In exemplary embodiments, all endogenous D segments are removed or replaced.

In some embodiments, camelid J gene segments are inserted within the animal genome in such a manner that some or all endogenous J gene segments are preserved. In exemplary embodiments, all endogenous J segments are preserved. In exemplary embodiments, at least 1 endogenous J segment is removed or replaced. In exemplary embodiments, at least 2 endogenous J segments are removed or replaced. In exemplary embodiments, at least 3 endogenous J segments are removed or replaced. In exemplary embodiments, at least 4 endogenous J segments are removed or replaced. In exemplary embodiments, all endogenous J segments are removed or replaced.

Alternatively, in some embodiments, the transgenic non-human animal of the present disclosure comprises an IgH locus comprising a) unrearranged heavy chain variable (V), diversity (D) and joining (J) gene segments comprising camelid D and/or J gene segments and b) at least one IgG constant region gene lacking a functional CH1 domain.

For example, the IgH locus of the transgenic non-human animal is modified by a) replacement of one or more endogenous non-human D and/or J gene segments for one or more unrearranged camelid D and/or J gene segments and b) partial or complete deletion of the CH1 domain of at least one IgG constant region gene.

Alternatively, the IgH locus of the transgenic non-human animal is modified by a) insertion of one or more unrearranged camelid D and/or J gene segments and b) partial or complete deletion of the CH1 domain of at least one IgG constant region gene.

In other embodiments, the IgH locus of the transgenic non-human animal is modified by a) replacement of one or more endogenous non-human D and/or J gene segments for one or more unrearranged camelid D and/or J gene segments or insertion of one or more unrearranged camelid D and/or J gene segments and b) modification of the CH1 domain of at least one IgG constant region gene.

In some embodiments, an IgH locus of the transgenic non-human animal is modified by replacement of all endogenous non-human D and J segments with non-human mammalian D and J gene segments. In some embodiments, an IgH locus of the transgenic non-human animal is modified by replacement of all endogenous non-human D and J segments with unrearranged non-human mammalian D and J gene segments.

In other embodiments, an IgH locus of the transgenic non-human animal is modified by replacement of all endogenous non-human D and J segments with unrearranged camelid D and J gene segments.

In some embodiments, at least one endogenous non-human D and/or J segments may be preserved.

In some embodiments, all endogenous non-human D and/or J segments may be preserved.

In an exemplary embodiment, the camelid D gene segments is from a single camelid species.

In another exemplary embodiment, the camelid D gene segments is from at least two camelid species.

In another exemplary embodiment, the camelid D gene segments is from at least three camelid species.

In another exemplary embodiment, the camelid D gene segments is from at least four camelid species.

In another exemplary embodiment, the camelid D gene segments is from at least five camelid species.

In an exemplary embodiment, the camelid J gene segments is from a single camelid species.

In another exemplary embodiment, the camelid J gene segments is from at least two camelid species.

In another exemplary embodiment, the camelid J gene segments is from at least three camelid species.

In another exemplary embodiment, the camelid J gene segments is from at least four camelid species.

In another exemplary embodiment, the camelid J gene segments is from at least five camelid species.

In accordance with the present disclosure, the camelid D and J gene segments is from a single camelid species.

Also in accordance with the present disclosure, the camelid D and J gene segments is from at least two camelid species.

Also in accordance with the present disclosure, the camelid D and J gene segments is from at least three camelid species.

In some embodiments, an IgH locus of the transgenic non-human animal is modified by replacement of one or more endogenous non-human V gene segments with V gene segments of multiple mammal species.

In some embodiments, an IgH locus of the transgenic non-human animal is modified by insertion of V gene segments of multiple mammal species.

Modifications of the IgH locus include for example, replacement of one or more endogenous non-human V gene segments with one or more camelid V gene segments or insertion of camelid V gene segments.

In some instances, all endogenous non-human V segments are replaced for camelid V gene segments.

Such replacement or insertion is usually carried out at an endogenous V site such that the camelid V gene segments are located in the same genomic area as the endogenous V segments.

In some embodiments, the transgenic non-human animal of the present disclosure comprises a) unrearranged heavy chain variable (V), diversity (D) and joining (J) gene segments comprising V, D and/or J gene segments from multiple camelid species and b) at least one IgG constant region gene lacking a functional CH1 domain.

In some embodiments, the IgG constant region gene of the transgenic non-human animal is an endogenous IgG constant region gene lacking a functional CH1 domain. However, in other embodiments, non-endogenous IgG constant region (e.g., a human IgG constant region or else) gene lacking a functional CH1 domain can also be used.

In some embodiments, the camelid V gene segments of the transgenic non-human animal is from a single camelid species.

Alternatively, in some embodiments, the camelid V gene segments are from at least two, at least three or at least four camelid species. Accordingly, in some embodiments, camelid V gene segments are from at least two camelid species. In some embodiments, camelid V gene segments are from at least three camelid species. In some embodiments, camelid V gene segments are from at least four camelid species. In some embodiments, camelid V gene segments are from at least five camelid species.

In some embodiments, the V gene segments encode VH polypeptides. In some particular embodiments, the VH is a camelid VH.

In other embodiments, the V gene segments encode VHH polypeptides. In some particular embodiments, the VHH is a camelid VHH.

In yet other embodiments of the disclosure the V gene segments encode VH and VHH polypeptides. In some particular embodiments, the VHs and VHHs are camelid VHs and VHHs.

In some embodiments, the camelid VHs and/or VHHs are from an alpaca, a llama, a Bactrian, a dromedary, a Vicunia or combination thereof. Accordingly, in some embodiments, the camelid VHs and/or VHHs comprise VHs and/or VHHs from an alpaca. In some embodiments, the camelid VHs and/or VHHs comprise VHs and/or VHHs from a llama. In some embodiments, the camelid VHs and/or VHHs comprise VHs and/or VHHs from a Bactrian. In some embodiments, the camelid VHs and/or VHHs comprise VHs and/or VHHs from a dromedary. In some embodiments, the camelid VHs and/or VHHs comprise VHs and/or VHHs from a Vicunia.

In some embodiments, the transgenic non-human animal comprises, for example, V segments from an alpaca, V segments from a Bactrian, V segments from a llama, and/or V segments from a dromedary, a Vicunia or combination thereof.

In some embodiments, the transgenic non-human animal comprises camelid D gene segments from an alpaca.

In some embodiments, the transgenic non-human animal comprises camelid D gene segments from a Bactrian.

In some embodiments, the transgenic non-human animal comprises camelid D gene segments from a llama.

In some embodiments, the transgenic non-human animal comprises camelid D gene segments from a dromedary.

In some embodiments, the transgenic non-human animal comprises camelid D gene segments from a Vicunia.

In some embodiments, the transgenic non-human animal comprises camelid J gene segments from an alpaca.

In some embodiments, the transgenic non-human animal comprises camelid J gene segments from a Bactrian.

In some embodiments, the transgenic non-human animal comprises camelid J gene segments from a llama.

In some embodiments, the transgenic non-human animal comprises camelid J gene segments from a dromedary.

In some embodiments, the transgenic non-human animal comprises camelid J gene segments from a Vicunia.

In some embodiments, the transgenic non-human animal comprises camelid D and J gene segments from an alpaca.

In some embodiments, the transgenic non-human animal comprises camelid D and J gene segments from a Bactrian.

In some embodiments, the transgenic non-human animal comprises camelid D and J gene segments from a llama.

In some embodiments, the transgenic non-human animal comprises camelid D and/or J gene segments from a dromedary.

In some embodiments, the transgenic non-human animal comprises camelid D and/or J gene segments from a Vicunia.

In some exemplary embodiments, the IgH locus of the transgenic animal disclosed herein comprises from one to at least seven (including 1, 2, 3, 4, 5, 6 or 7) alpaca D gene segments. In some embodiments, the IgH locus of the transgenic animal disclosed herein comprises 1 alpaca D gene segment. In some embodiments, the IgH locus of the transgenic animal disclosed herein comprises 2 alpaca D gene segments. In some embodiments, the IgH locus of the transgenic animal disclosed herein comprises 3 alpaca D gene segments. In some embodiments, the IgH locus of the transgenic animal disclosed herein comprises 4 alpaca D gene segments. In some embodiments, the IgH locus of the transgenic animal disclosed herein comprises 5 alpaca D gene segments. In some embodiments, the IgH locus of the transgenic animal disclosed herein comprises 6 alpaca D gene segments. In some embodiments, the IgH locus of the transgenic animal disclosed herein comprises 7 alpaca D gene segments. In some embodiments, the IgH locus of the transgenic animal disclosed herein comprises more than 7 alpaca D gene segments.

In other exemplary embodiments, the IgH locus of the transgenic animal disclosed herein comprises from one to at least seven (including 1, 2, 3, 4, 5, 6 or 7) alpaca J gene segments. In some embodiments, the IgH locus of the transgenic animal disclosed herein comprises 1 alpaca J gene segment. In some embodiments, the IgH locus of the transgenic animal disclosed herein comprises 2 alpaca J gene segments. In some embodiments, the IgH locus of the transgenic animal disclosed herein comprises 3 alpaca J gene segments. In some embodiments, the IgH locus of the transgenic animal disclosed herein comprises 4 alpaca J gene segments. In some embodiments, the IgH locus of the transgenic animal disclosed herein comprises 5 alpaca J gene segments. In some embodiments, the IgH locus of the transgenic animal disclosed herein comprises 6 alpaca J gene segments. In some embodiments, the IgH locus of the transgenic animal disclosed herein comprises 7 alpaca J gene segments. In some embodiments, the IgH locus of the transgenic animal disclosed herein comprises more than 7 alpaca J gene segments.

In yet other exemplary embodiments, the IgH locus of the transgenic animal disclosed herein comprises from one to at least seven (including 1, 2, 3, 4, 5, 6 or 7) Bactrian D gene segments. In some embodiments, the IgH locus of the transgenic animal disclosed herein comprises 1 Bactrian D gene segment. In some embodiments, the IgH locus of the transgenic animal disclosed herein comprises 2 Bactrian D gene segments. In some embodiments, the IgH locus of the transgenic animal disclosed herein comprises 3 Bactrian D gene segments. In some embodiments, the IgH locus of the transgenic animal disclosed herein comprises 4 Bactrian D gene segments. In some embodiments, the IgH locus of the transgenic animal disclosed herein comprises 5 Bactrian D gene segments. In some embodiments, the IgH locus of the transgenic animal disclosed herein comprises 6 Bactrian D gene segments. In some embodiments, the IgH locus of the transgenic animal disclosed herein comprises 7 Bactrian D gene segments. In some embodiments, the IgH locus of the transgenic animal disclosed herein comprises more than 7 Bactrian D gene segments.

In some exemplary embodiments, the IgH locus of the transgenic animal disclosed herein comprises from one to at least seven (including 1, 2, 3, 4, 5, 6, 7 or more than 7) Bactrian J gene segments. In some embodiments, the IgH locus of the transgenic animal disclosed herein comprises 1 Bactrian J gene segment. In some embodiments, the IgH locus of the transgenic animal disclosed herein comprises 2 Bactrian J gene segments. In some embodiments, the IgH locus of the transgenic animal disclosed herein comprises 3 Bactrian J gene segments. In some embodiments, the IgH locus of the transgenic animal disclosed herein comprises 4 Bactrian J gene segments. In some embodiments, the IgH locus of the transgenic animal disclosed herein comprises 5 Bactrian J gene segments. In some embodiments, the IgH locus of the transgenic animal disclosed herein comprises 6 Bactrian J gene segments. In some embodiments, the IgH locus of the transgenic animal disclosed herein comprises 7 Bactrian J gene segments. In some embodiments, the IgH locus of the transgenic animal disclosed herein comprises more than 7 Bactrian J gene segments.

In other exemplary embodiments, the IgH locus of the transgenic animal disclosed herein comprises from one to at least seven (including 1, 2, 3, 4, 5, 6, 7 or more than 7) alpaca V gene segments. In some embodiments, the IgH locus of the transgenic animal disclosed herein comprises 1 alpaca V gene segment. In some embodiments, the IgH locus of the transgenic animal disclosed herein comprises 2 alpaca V gene segments. In some embodiments, the IgH locus of the transgenic animal disclosed herein comprises 3 alpaca V gene segments. In some embodiments, the IgH locus of the transgenic animal disclosed herein comprises 4 alpaca V gene segments. In some embodiments, the IgH locus of the transgenic animal disclosed herein comprises 5 alpaca V gene segments. In some embodiments, the IgH locus of the transgenic animal disclosed herein comprises 6 alpaca V gene segments. In some embodiments, the IgH locus of the transgenic animal disclosed herein comprises 7 alpaca V gene segments. In some embodiments, the IgH locus of the transgenic animal disclosed herein comprises more than 7 alpaca V gene segments.

In additional exemplary embodiments, the IgH locus of the transgenic animal disclosed herein comprises from one to at least ten (including 1, 2, 3, 4, 5, 6, 7, 8, 9, 10 or more than 10) Bactrian V gene segments. In some embodiments, the IgH locus of the transgenic animal disclosed herein comprises 1 Bactrian V gene segment. In some embodiments, the IgH locus of the transgenic animal disclosed herein comprises 2 Bactrian V gene segments. In some embodiments, the IgH locus of the transgenic animal disclosed herein comprises 3 Bactrian V gene segments. In some embodiments, the IgH locus of the transgenic animal disclosed herein comprises 4 Bactrian V gene segments. In some embodiments, the IgH locus of the transgenic animal disclosed herein comprises 5 Bactrian V gene segments. In some embodiments, the IgH locus of the transgenic animal disclosed herein comprises 6 Bactrian V gene segments. In some embodiments, the IgH locus of the transgenic animal disclosed herein comprises 7 Bactrian V gene segments. In some embodiments, the IgH locus of the transgenic animal disclosed herein comprises 8 Bactrian V gene segments. In some embodiments, the IgH locus of the transgenic animal disclosed herein comprises 9 Bactrian V gene segments. In some embodiments, the IgH locus of the transgenic animal disclosed herein comprises 10 Bactrian V gene segments. In some embodiments, the IgH locus of the transgenic animal disclosed herein comprises more than 10 Bactrian V gene segments.

In other exemplary embodiments, the IgH locus of the transgenic animal disclosed herein comprises from one to at least ten (including 1, 2, 3, 4, 5, 6, 7, 8, 9, 10 or more than 10) llama V gene segments. In some embodiments, the IgH locus of the transgenic animal disclosed herein comprises 1 llama V gene segment. In some embodiments, the IgH locus of the transgenic animal disclosed herein comprises 2 llama V gene segments. In some embodiments, the IgH locus of the transgenic animal disclosed herein comprises 3 llama V gene segments. In some embodiments, the IgH locus of the transgenic animal disclosed herein comprises 4 llama V gene segments. In some embodiments, the IgH locus of the transgenic animal disclosed herein comprises 5 llama V gene segments. In some embodiments, the IgH locus of the transgenic animal disclosed herein comprises 6 llama V gene segments. In some embodiments, the IgH locus of the transgenic animal disclosed herein comprises 7 llama V gene segments. In some embodiments, the IgH locus of the transgenic animal disclosed herein comprises 8 llama V gene segments. In some embodiments, the IgH locus of the transgenic animal disclosed herein comprises 9 llama V gene segments. In some embodiments, the IgH locus of the transgenic animal disclosed herein comprises 10 llama V gene segments. In some embodiments, the IgH locus of the transgenic animal disclosed herein comprises more than 10 llama V gene segments.

In some exemplary embodiments, the IgH locus of the transgenic animal disclosed herein comprises from one to at least seven (including 1, 2, 3, 4, 5, 6, 7 or more than 7) dromedary V gene segments. In some embodiments, the IgH locus of the transgenic animal disclosed herein comprises 1 dromedary V gene segment. In some embodiments, the IgH locus of the transgenic animal disclosed herein comprises 2 dromedary V gene segments. In some embodiments, the IgH locus of the transgenic animal disclosed herein comprises 3 dromedary V gene segments. In some embodiments, the IgH locus of the transgenic animal disclosed herein comprises 4 dromedary V gene segments. In some embodiments, the IgH locus of the transgenic animal disclosed herein comprises 5 dromedary V gene segments. In some embodiments, the IgH locus of the transgenic animal disclosed herein comprises 6 dromedary V gene segments. In some embodiments, the IgH locus of the transgenic animal disclosed herein comprises 7 dromedary V gene segments. In some embodiments, the IgH locus of the transgenic animal disclosed herein comprises more than 7 dromedary V gene segments.

In some exemplary embodiments, the IgH locus of the transgenic animal disclosed herein comprises from one to at least seven (including 1, 2, 3, 4, 5, 6, 7 or more than 7) Vicunia V gene segments. In some embodiments, the IgH locus of the transgenic animal disclosed herein comprises 1 Vicunia V gene segment. In some embodiments, the IgH locus of the transgenic animal disclosed herein comprises 2 Vicunia V gene segments. In some embodiments, the IgH locus of the transgenic animal disclosed herein comprises 3 Vicunia V gene segments. In some embodiments, the IgH locus of the transgenic animal disclosed herein comprises 4 Vicunia V gene segments. In some embodiments, the IgH locus of the transgenic animal disclosed herein comprises 5 Vicunia V gene segments. In some embodiments, the IgH locus of the transgenic animal disclosed herein comprises 6 Vicunia V gene segments. In some embodiments, the IgH locus of the transgenic animal disclosed herein comprises 7 Vicunia V gene segments. In some embodiments, the IgH locus of the transgenic animal disclosed herein comprises more than 7 Vicunia V gene segments.

In some embodiments, the V gene segments, D gene segments and/or J gene segments encode a naturally occurring sequence.

In other embodiments, the V gene segments, D gene segments and/or J gene segments encode a mutated or non-naturally occurring sequence.

In some exemplary embodiments, the IgG constant region gene of the transgenic animal is the endogenous non-human IgG constant region gene or a portion thereof.

In accordance with the present disclosure, the IgG constant region gene lacking a functional CH1 domain may be a mouse γ3 constant region gene, a mouse γ1 constant region gene, a mouse γ2b constant region gene or a mouse γ2a constant region gene.

In accordance with the present disclosure, the IgG constant region gene lacking a functional CH1 domain may be a rat γ1 constant region gene, a rat γ2b constant region gene, a rat γ2a constant region gene or a rat γ2c constant region gene.

For example, in some embodiments, the IgH locus of the transgenic animal comprises a γ3 constant region gene comprising a partial or complete deletion in the region encoding the CH1 domain in one or both alleles.

Alternatively, in some embodiments, the IgH locus of the transgenic animal comprises a yl constant region gene comprising a partial or complete deletion in the region encoding the CH1 domain in one or both alleles.

Moreover, in some embodiments, the IgH locus of the transgenic animal comprises a γ2b constant region gene comprising a partial or complete deletion in the region encoding the CH1 domain in one or both alleles.

Also, in some embodiments the IgH locus of the transgenic animal comprises a γ2a constant region gene comprising a partial or complete deletion in the region encoding the CH1 domain in one or both alleles.

Also, in other embodiments the IgH locus of the transgenic animal comprises a γ2c constant region gene comprising a partial or complete deletion in the region encoding the CH1 domain in one or both alleles.

In some embodiments, the transgenic animal comprises at least some endogenous gamma globulin genes that are identical to that of a non-transgenic animal counterpart. In some embodiments, at least one or all of the endogenous gamma globulin genes are modified to allow expression of HCAbs.

In some exemplary embodiments, at least two IgG constant region genes of the transgenic animal comprise a partial or complete deletion in the region encoding the CH1 domain.

In other exemplary embodiments, at least three IgG constant region genes of the transgenic animal comprise a partial or complete deletion in the region encoding the CH1 domain.

In further exemplary embodiments, all IgG constant region genes of the transgenic animal comprise a partial or complete deletion in the region encoding the CH1 domain.

In other exemplary embodiments, at least one IgG constant region gene of the transgenic animal comprises a partial or complete deletion in the region encoding the CH1 domain and at least one other IgG constant region gene is completely or partially deleted.

For example, the genome of the transgenic non-human animal of the present disclosure may have at least one allele having a γ3 constant region gene comprising a partial or complete deletion in the region encoding the CH1 domain, a γ1 constant region gene comprising a partial or complete deletion in the region encoding the CH1 domain, a γ2b constant region gene comprising a partial or complete deletion in the region encoding the CH1 domain, and/or a γ2a constant region gene comprising a partial or complete deletion in the region encoding the CH1 domain or combination thereof.

In other exemplary embodiments, one allele of the transgenic non-human animal genome comprises a partial or complete deletion of the γ3 and γ2b constant region genes and the γ1 and γ2a constant region genes comprise a partial or complete deletion in the region encoding the CH1 domain.

In yet other exemplary embodiments, one allele of the transgenic non-human animal genome comprises an IgH locus comprising a γ2b constant region gene comprising a partial or complete deletion in the region encoding the CH1 domain.

In yet other exemplary embodiments, one allele of the transgenic non-human animal genome comprises an IgH locus comprising a γ3 constant region gene comprising a partial or complete deletion in the region encoding the CH1 domain and optionally a γ2a constant region gene comprising a partial or complete deletion in the region encoding the CH1 domain and/or a γ2b constant region gene comprising a partial or complete deletion in the region encoding the CH1 domain.

The modifications in the IgH locus may occur in one or both alleles. As such, in some embodiments, the other allele of the transgenic non-human animal genome comprises an identical IgH locus or an identical IgG constant region gene. Alternatively, in other embodiments, the IgH locus of both alleles is different. In some embodiments, each allele carries different modifications at an IgH locus or in the IgG constant region gene.

In some embodiments, the other allele of the transgenic non-human animal genome comprises a wild type IgH locus or a wild type IgG constant region gene.

As such, in some exemplary embodiments, the other allele comprises wild type endogenous γ3, γ1, γ2b and γ2a constant region genes.

In other exemplary embodiments, the other allele of the transgenic non-human animal genome comprises an IgH locus comprising a modification selected from a partial or complete deletion in the region encoding the CH1 domain of at least one or all IgG constant region genes, a complete or partial deletion of at least one or all other IgG constant region genes or a combination thereof.

In some embodiments, the other allele comprises a complete deletion of the γ3, γ1 and γ2b constant region genes and a γ2a constant region gene comprising a partial or complete deletion in the region encoding the CH1 domain.

In other embodiments, the other allele comprises a γ3 and γ2a constant region gene comprising a partial or complete deletion in the region encoding the CH1 domain and a partial or complete deletion of the γ2b constant region gene.

In additional embodiments, the other allele comprises a partial or complete deletion of the γ3, γ1 and γ2b constant region gene and a γ2a constant region gene comprising a partial or complete deletion in the region encoding the CH1 domain.

In other embodiments, the other allele comprises a γ3 constant region gene comprising a partial or complete deletion in the region encoding the CH1 domain.

In yet other embodiments, the other allele comprises a partial or complete deletion of the γ3, γ1 and γ2b constant region genes.

In some embodiments, the other allele comprises a γ3 and γ2a constant region gene comprising a partial or complete deletion in the region encoding the CH1 domain and a partial or complete deletion of γ2b constant region gene.

In other exemplary embodiments, both alleles of the transgenic non-human animal genome comprise an IgH locus comprising a γ2b constant region gene comprising a partial or complete deletion in the region encoding the CH1 domain.

In yet other exemplary embodiments, both alleles of the transgenic non-human animal genome comprise an IgH locus comprising γ3 and γ2a constant region genes comprising a partial or complete deletion in the region encoding the CH1 domain and a partial or complete deletion of γ2b constant region gene.

In yet an additional exemplary embodiment, both alleles of the transgenic non-human animal genome comprise an IgH locus comprising γ3, γ1, γ2b and γ2a constant region genes comprising a partial or complete deletion in the region encoding the CH1 domain.

In another exemplary embodiment, both alleles of the transgenic non-human animal genome comprise an IgH locus comprising γ3, γ1, γ2b and γ2a constant region genes comprising a complete deletion in the region encoding the CH1 domain.

In other aspects and embodiments, the transgenic non-human animal IgH locus comprises at least one different V gene segment on each allele.

Also in accordance with the present disclosure, the transgenic non-human animal IgH locus comprises at least one V gene segment of one species in one of its alleles and at least one V gene segment of another species in the other allele.

In aspects and embodiments of the present disclosure, the transgenic non-human animal comprises a germline modification of an IgM constant region gene. The modification comprises, for example, replacement of the IgM CH1 domain for a camelid CH1 domain.

In other aspects and embodiments of the present disclosure, the transgenic non-human animal comprises a germline modification of an IgA constant region gene. The modification comprises, for example, replacement of the IgA CH1 domain for a camelid CH1 domain.

In yet other aspects and embodiments of the present disclosure, the transgenic non-human animal comprises a germline modification of an IgE constant region gene. The modification comprises, for example, replacement of the IgE CH1 domain for a camelid CH1 domain.

In further aspects and embodiments of the present disclosure, the transgenic non-human animal comprises a germline modification of an IgD constant region gene. The modification comprises, for example, replacement of the IgD CH1 domain for a camelid CH1 domain.

In accordance with the present disclosure, the transgenic non-human animal comprises at least about 10 kb of camelid V gene segments of llama, Bactrian and/or alpaca species.

In accordance with the present disclosure, the transgenic non-human animal comprises at least about 20 kb of camelid V gene segments of llama, Bactrian and/or alpaca species.

In accordance with the present disclosure, the transgenic non-human animal comprises at least about 30 kb of camelid V gene segments of llama, Bactrian and/or alpaca species.

In accordance with the present disclosure, the transgenic non-human animal comprises at least about 40 kb of camelid V gene segments of llama, Bactrian and/or alpaca species.

In accordance with the present disclosure, the transgenic non-human animal comprises at least about 50 kb of camelid V gene segments of llama, Bactrian and/or alpaca species.

In some embodiments, the transgenic non-human animal comprises less than 50 kb of camelid V gene segments of llama, Bactrian and/or alpaca species.

Further in accordance with the present disclosure, the V gene segments, D gene segments and J gene segments are capable of VDJ rearrangement. For example, the endogenous V gene segments may rearrange with camelid D and J segments. Alternatively, the camelid V gene segments may rearrange to camelid D and J.

In some embodiments, the transgenic non-human animal is heterozygous.

In other embodiments, the transgenic non-human animal is homozygous.

In exemplary embodiments, the transgenic non-human animal is a transgenic rat.

In other exemplary embodiments, the transgenic non-human animal is a transgenic mouse.

In accordance with exemplary embodiments the present disclosure, the transgenic non-human animal is a transgenic mouse having an IgH locus comprising an IgG constant region gene encoding a mouse IgG1, a mouse IgG2a, a mouse IgG2b or a mouse IgG3 constant region lacking a CH1 domain.

In an exemplary embodiment, the transgenic mouse has at least two of its IgG constant regions selected from the constant region of an IgG1 gene, an IgG2a gene, an IgG2b gene or an IgG3 gene that lack a CH1 domain.

In another exemplary embodiment, the transgenic mouse has at least three of its IgG constant regions selected from the constant region of an IgG1 gene, an IgG2a gene, an IgG2b gene or an IgG3 gene that lack a CH1 domain.

In yet another exemplary embodiment, each of the transgenic mouse IgG1 gene, IgG2a gene, IgG2b gene or IgG3 gene lack a CH1 domain.

In another exemplary embodiment, the transgenic mouse has at least one of its IgG1 gene, IgG2a gene, IgG2b gene or IgG3 gene partially or completely deleted.

In some exemplary embodiments, all endogenous mouse D and J gene segments of the transgenic mouse are replaced with unrearranged camelid D and J gene segments.

In other exemplary embodiments, the IgH locus of the transgenic mouse comprises one or more mouse V gene segments and unrearranged camelid V gene segments.

In yet other exemplary embodiments, all endogenous mouse V gene segments of the transgenic mouse are replaced with unrearranged camelid V gene segments.

In other embodiments, the transgenic mouse has at least one endogenous mouse IgG constant region gene lacking a functional CH1 domain.

In additional embodiments, all endogenous mouse IgG constant region genes of one allele of the transgenic mouse lack a functional CH1 domain.

In other embodiments, all endogenous mouse IgG constant region genes of both alleles of the transgenic mouse lack a functional CH1 domain.

In some exemplary embodiments, the transgenic mouse is heterozygous and has one allele comprising a partial or complete deletion in the region encoding the CH1 domain of at least one IgG constant region genes and optionally a complete or partial deletion of at least one other IgG constant region genes and the other allele is wild type.

Also, in other exemplary embodiments, the transgenic mouse is heterozygous and has one allele comprising a partial or complete deletion in the region encoding the CH1 domain of at least one IgG constant region genes and optionally a complete or partial deletion of at least one or all other IgG constant region genes and the other allele optionally comprises a partial or complete deletion in the region encoding the CH1 domain of at least one IgG constant region genes or a complete or partial deletion of at least one or all other IgG constant region genes or a combination thereof.

In other exemplary embodiments, the transgenic mouse is homozygous.

In further exemplary embodiments, the transgenic non-human animal of the present disclosure is a transgenic mouse having a germline modification at the IgH locus comprising a) replacement of the endogenous mouse D and J gene segments for unrearranged camelid D and J gene segments a) replacement of one or more of the endogenous mouse V gene segments for one or more unrearranged camelid V gene segments or insertion of one or more unrearranged camelid V gene segments and c) deletion of the CH1 domain of at least one or all of endogenous mouse γ1, γ2a, γ2b and γ3 gene so that a polypeptide expressed from said endogenous mouse γ1, γ2a, γ2b and γ3 gene does not comprise a functional CH1 domain.

In other exemplary embodiments, the transgenic non-human animal of the present disclosure is a transgenic mouse having a germline modification at the IgH locus comprising a) insertion of unrearranged camelid D and/or J gene segments at an endogenous mouse D and/or J site a) replacement of one or more of the endogenous mouse V gene segments for one or more unrearranged camelid V gene segments or insertion of one or more unrearranged camelid V gene segments and c) deletion of the CH1 domain of at least one or all of endogenous mouse γ1, γ2a, γ2b and γ3 gene so that a polypeptide expressed from said endogenous mouse γ1, γ2a, γ2b and γ3 gene does not comprise a functional CH1 domain.

In another exemplary embodiments, the transgenic non-human animal of the present disclosure is a transgenic mouse having a germline modification at the IgH locus comprising a) replacement of the endogenous mouse D and J gene segments for unrearranged camelid D and J gene segments a) replacement of one or more of the endogenous mouse V gene segments for one or more unrearranged camelid V gene segments or insertion of one or more unrearranged camelid V gene segments and c) modification of the CH1 domain of at least one or all of endogenous mouse yl, γ2a, γ2b and γ3 gene so that a polypeptide expressed from said endogenous mouse γ1, γ2a, γ2b and γ3 gene does not comprise a functional CH1 domain.

In another exemplary embodiments, the transgenic non-human animal of the present disclosure is a transgenic mouse comprising germline modifications at an immunoglobulin heavy chain (IgH) locus, wherein the modification comprises deletion of the CH1 domain of each of the endogenous mouse γ3 gene, γ1 gene, γ2b gene and γ2a gene, replacement of mouse D and J gene segments for unrearranged camelid D and J gene segments, insertion of camelid V gene segments from multiple camelid species and optionally deletion of at least one or all endogenous mouse V gene segments.

In some embodiments, the camelid V segments encodes camelid VH and camelid VHH polypeptides.

In some embodiments the transgenic non-human animal is capable of expressing heavy chain only antibodies (HCAbs) or nucleic acids encoding same following immunization with an antigen.

In some embodiments the camelid V segments encodes VH and/or VHH polypeptides from an alpaca, a Bactrian and a llama.

In some embodiments the camelid V segments encodes VH and/or VHH polypeptides from an alpaca, a Bactrian, a llama and a dromedary.

In some embodiments the camelid V segments encodes VH and/or VHH polypeptides from an alpaca, a Bactrian, a llama, a Vicunia and a dromedary.

In exemplary embodiments, the transgenic mouse has an MHC haplotype characterized as H-2^{b}.

In other exemplary embodiments, the transgenic mouse has the genetic background of a C57BL/6 mouse strain.

In some embodiments, the transgenic mouse has the genetic background of an inbred strain including for example and without limitations C3H, FVB or 129/Sv.

In some embodiments, the transgenic mouse has the genetic background of an outbred strain including for example and without limitations CD-1 or CF-1.

In another aspect, the present disclosure relates to a method for obtaining a heavy chain only antibody (HCAb) or an antigen-binding domain of a HCAb, nucleic acids encoding a HCAb, an antigen-binding domain of the HCAb or a portion thereof.

In exemplary embodiments, the method comprises immunizing the transgenic non-human animal disclosed herein with an antigen.

The transgenic non-human animal may produce a plurality of HCAbs upon immunization with the antigen. The plurality of HCAbs may comprise, for example, at least one HCAb species comprising a V portion encoded by a V segment of a first camelid species and a second HCAb species comprising V portion encoded by a V segment of a second camelid species.

In some embodiments, the method of the present disclosure comprises a step of collecting total RNA or messenger RNAs from the transgenic non-human animal.

In some embodiments, serum sample and/or spleen tissues are collected and RNAs are extracted to construct one or more library of variable heavy chains (VHs).

In some embodiments, the HCAbs are selected based on their binding property toward the antigen.

In some embodiments, the method comprises a step of determining the amino acid sequence or nucleic acid sequence of one or more complementarity determining regions or variable region of the HCAb species.

In an exemplary embodiment, the method is computer-based and comprises a software for organizing the sequence information in clusters based on predetermined parameters.

In some embodiments, the method of the present disclosure comprises a step of selecting one or more sequences of a HCAb to make a binding agent.

Exemplary embodiments of binding agents include for example and without limitations, an antibody (including bi-, tri-, multi-specific antibody), a single domain antibody, a single chain Fv, a chimeric antigen receptor (CAR), a bispecific T cell engager construct (BiTE), a bispecific killer cell engager (BiKE), a trispecific killer cell engager (TriKE) or an antigen binding fragment thereof.

In an exemplary embodiment, the binding agent as a format as set forth in U.S. Provisional appl. No. 62/951,701 and in PCT/CA2020/051753 published on Jun. 24, 2021 under number WO2021119832A1 or as described in Deyev, S. M et al. (BioEssays 30:904-918, 2008), the entire content of all of which is incorporated herein by reference.

In some embodiments, the binding agent comprises, for example, an endogenous VH portion.

In some embodiments, the binding agent comprises, for example, a camelid VHH portion.

In some embodiments, the binding agent comprises, for example, a camelid VH portion.

In some embodiments, the binding agent comprises, for example, a camelid D portion.

In some embodiments, the binding agent comprises, for example, a camelid J portion.

In some embodiments the binding agent is a multivalent and/or multi-specific antibody.

Also, in accordance with the present disclosure, the method may be carried out on a transgenic mouse comprising germline modifications at an IgH locus comprising a) replacement of one or more of the endogenous mouse V gene segments for one or more unrearranged camelid V gene segments or insertion of unrearranged camelid V gene segments, b) replacement of at least one or all of the endogenous mouse D and J segments with camelid D and J segments and c) deletion or modification of the CH1 domain of at least one or all of endogenous mouse γ1, γ2a, γ2b or γ3 gene so that a polypeptide expressed from said endogenous mouse γ1, γ2a, γ2b or γ3 gene does not comprise a functional CH1 domain.

Methods for making a binding agent are also encompassed by the present disclosure.

In some embodiments, the method comprises immunizing the transgenic non-human animal of the present disclosure with an antigen, obtaining the amino acid sequence or nucleic acid sequence of one or more complementarity determining regions or variable region of at least one HCAb species and generating a binding agent comprising the amino acid sequence.

In an exemplary embodiment, the amino acid sequence or nucleic acid sequence of one or more complementarity determining regions or variable region of a plurality of HCAb species is obtained and a binding agent comprising a most representative or a common sequence is generated.

In another exemplary embodiment, the amino acid sequence or nucleic acid sequence of one or more complementarity determining regions or variable region of a plurality of HCAb species is obtained and a binding agent comprising a least represented or a unique sequence is generated.

The present disclosure also relates to a binding agent comprising an amino acid sequence or encoded by a nucleic acid sequence obtained by the method disclosed herein or isolated or obtained from the transgenic non-human animal disclosed herein.

The present disclosure also relates to a binding agent comprising an amino acid sequence or encoded by a nucleic acid sequence obtained by immunizing the transgenic non-human animal disclosed herein with an antigen.

In some embodiments, the antigen is an antigen expressed by human cells.

In some embodiments, the antigen is a tumor antigen.

In some embodiments, the antigen is a checkpoint protein.

In some embodiments, the antigen is a protein expressed at the surface of immune cells.

In some embodiment, the antigen is from a pathogen and includes for example and without limitations, bacterial antigens, viral antigens, parasite antigens.

The present disclosure also relates to a nucleic acid construct for targeted replacement of gene segments at an IgH locus.

The present disclosure also relates to a nucleic acid construct for targeted insertion of gene segments at an IgH locus.

In some embodiments, the nucleic acid construct of the present disclosure comprises genomic non-human D and/or J segments. In some embodiments, the nucleic acid construct of the present disclosure comprises genomic human D and/or J segments.

In some embodiments, the nucleic acid construct of the present disclosure comprises genomic camelid D and/or J segments.

In some embodiments, the nucleic acid construct of the present disclosure comprises genomic endogenous mouse D and/or J segments.

In some embodiments, the nucleic acid construct of the present disclosure comprises genomic endogenous mouse D and/or J segments and genomic camelid D and/or J segments.

In some embodiments, the nucleic acid construct of the present disclosure comprises genomic non-human V, D and/or J segments. In some embodiments, the nucleic acid construct of the present disclosure comprises genomic human V, D and/or J segments.

In other embodiments, the nucleic acid construct of the present disclosure comprises genomic camelid V, D and/or J segments.

In an embodiment, the nucleic acid construct is a DNA construct.

In accordance with the present disclosure, the DNA construct comprises genomic camelid V segments and introns comprising recombination signal sequences for VDJ rearrangement.

In an exemplary embodiment, the DNA construct comprises camelid V segments from at least one species.

In another exemplary embodiment, the DNA construct comprises camelid V segments from at least two species.

In yet another exemplary embodiment, the DNA construct comprises camelid V segments from at least three species.

In yet another exemplary embodiment, the DNA construct comprises camelid V segments from at least four species.

In yet another exemplary embodiment, the DNA construct comprises camelid V segments from at least five species.

In accordance with the present disclosure, the camelid V segments encode camelid VH or camelid VHH polypeptides.

In an additional exemplary embodiment, the DNA construct comprises D and J segments from one camelid species.

In an additional exemplary embodiment, the DNA construct comprises D and J segments from two camelid species.

In an additional exemplary embodiment, the DNA construct comprises D and J segments from three camelid species.

In an additional exemplary embodiment, the DNA construct comprises D and J segments from four camelid species.

In an additional exemplary embodiment, the DNA construct comprises D and J segments from five camelid species.

In a further exemplary embodiment, the DNA construct comprises from one to at least seven D gene segments of alpacas.

In another exemplary embodiment, the DNA construct comprises from one to at least seven J gene segments of alpacas.

In another exemplary embodiment, the DNA construct comprises from one to at least seven Bactrian D gene segments.

In a further exemplary embodiment, the DNA construct comprises from one to at least seven Bactrian J gene segments.

In another exemplary embodiment, the DNA construct comprises from one to at least seven Llama D gene segments.

In a further exemplary embodiment, the DNA construct comprises from one to at least seven Llama J gene segments.

In another exemplary embodiment, the DNA construct comprises from one to at least seven dromedaries D gene segments.

In a further exemplary embodiment, the DNA construct comprises from one to at least seven dromedaries J gene segments.

In another exemplary embodiment, the DNA construct comprises from one to at least seven Vicunia D gene segments.

In a further exemplary embodiment, the DNA construct comprises from one to at least seven Vicunia J gene segments.

In yet a further exemplary embodiment, the DNA construct comprises from one to at least ten alpaca V gene segments.

In an additional exemplary embodiment, the DNA construct comprises from one to at least ten Bactrians V gene segments.

In yet an additional exemplary embodiment, the DNA construct comprises from one to at least ten llama V gene segments.

In a further exemplary embodiment, the DNA construct comprises from one to at least ten dromedaries V gene segments.

In a further exemplary embodiment, the DNA construct comprises from one to at least ten Vicunia V gene segments.

In an exemplary embodiment, the DNA construct comprises in a 5′ to 3′ fashion mouse VH segments, alpaca VH and/or VHH segments, alpaca D segments and alpaca J segments.

In another exemplary embodiment, the DNA construct comprises in a 5′ to 3′ fashion mouse VH segments, Bactrian VH and/or VHH segments, alpaca VH and/or VHH segments, alpaca D segments and alpaca J segments.

In another exemplary embodiment, the DNA construct comprises in a 5′ to 3′ fashion mouse VH segments, llama VH and/or VHH segments, alpaca VH and/or VHH segments, alpaca D segments and alpaca J segments.

In further exemplary embodiment, the DNA construct comprises in a 5′ to 3′ fashion mouse VH segments, llama VH and/or VHH segments, Bactrian VH and/or VHH segments, alpaca VH and/or VHH segments, alpaca D segments and alpaca J segments.

In another exemplary embodiment, the DNA construct comprises in a 5′ to 3′ fashion mouse VH segments, Bactrian VH and/or VHH segments, llama VH and/or VHH segments, alpaca VH and/or VHH segments, alpaca D segments and alpaca J segments.

In yet another exemplary embodiment, the DNA construct comprises in a 5′ to 3′ fashion mouse VH segments, llama VH and/or VHH segments, Bactrian VH and/or VHH segments, alpaca VH and/or VHH segments, alpaca D segments, Bactrian D segments, Bactrian J segments, and alpaca J segments.

In an exemplary embodiment, the DNA construct comprises in a 5′ to 3′ fashion mouse VH segments, llama VH and/or VHH segments, Bactrian VH and/or VHH segments, alpaca VH and/or VHH segments, Bactrian D segments and Bactrian J segments.

In another exemplary embodiment, the DNA construct comprises in a 5′ to 3′ fashion, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

In yet another exemplary embodiment, the DNA construct comprises in a 5′ to 3′ fashion, alpaca VH and/or VHH segments, Bactrian VH and/or VHH segments, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

In accordance with the present disclosure, the DNA construct is provided as an artificial chromosome. For example, in some embodiments, the DNA construct is provided as a bacterial artificial chromosome (BAC). In some embodiments, the DNA construct is provided as a yeast artificial chromosome (YAC). In some embodiments, the DNA construct is provided as a mammalian artificial chromosome (MAC).

The present disclosure also relates to the use of the DNA construct disclosed herein for modifying embryonic non-human stem cells or for making a transgenic non-human animal.

The present disclosure also relates to isolated embryonic non-human stem cells modified by the DNA construct disclosed herein.

In exemplary embodiments, the isolated embryonic non-human stem cells of the present disclosure comprise germline modifications at an immunoglobulin heavy chain (IgH) locus that comprise a) unrearranged heavy chain variable (V), diversity (D) and joining (J) gene segments and wherein the D and/or J gene segments comprise camelid D and/or J gene segments and b) at least one IgG constant region gene lacking a functional CH1 domain.

The modifications are performed on the endogenous IgG constant region.

The embryonic non-human stem cells disclosed herein may be used for making a transgenic non-human animal.

The present disclosure also provides a process for producing a transgenic non-human animal.

In some embodiments, the process comprises the steps of injecting the embryonic non-human stem cells disclosed herein into a mouse blastocyst, implanting the mouse blastocysts or embryo into a pseudopregnant mouse and selecting the mouse progeny carrying the germline modifications.

The present disclosure also relates to a cell isolated from the transgenic non-human animal disclosed herein.

In some aspects, methods of making a transgenic animal are provided comprising use of the nucleic acid construct as described herein.

In some embodiments, the method of making a transgenic animal comprises introducing a nucleic acid construct into a stem cell, the nucleic acid comprising a genomic camelid D and/or J segments and optionally comprises genomic camelid V segments and wherein the nucleic acid construct comprises introns comprising recombination signal sequences for VDJ rearrangement.

In other exemplary embodiments, the method of making a transgenic animal comprises implanting a pseudopregnant mouse with a blastocyst microinjected with the genetically modified embryonic stem cells disclosed herein.

In other exemplary embodiments, the method of making a transgenic animal comprises implanting a pseudopregnant mouse with a blastocyst microinjected with the embryonic stem cells genetically modified with the nucleic acid construct disclosed herein.

In some embodiment, the method may comprise selecting chimeric mice from litter.

In some embodiment, the method may comprise generating F1 heterozygous animals by backcrossing a chimeric mouse with a wild type mouse.

In some embodiments, the method may comprise generating F2 homozygous animals by crossing F1 animals.

For example, blastocyst microinjected with embryonic stem cells genetically modified with the nucleic acid construct disclosed herein are implanted into a pseudopregnant mouse, chimeric mice are selected from litter and optionally F1 heterozygous animals are generated by backcrossing a chimeric mouse with a wild type mouse and optionally F2 homozygous animals are generated by crossing F1 animals.

In another example, blastocyst microinjected the embryonic stem cells disclosed herein are implanted into a pseudopregnant mouse, chimeric mice are selected from litter and optionally F1 heterozygous animals are generated by backcrossing a chimeric mouse with a wild type mouse and optionally F2 homozygous animals are generated by crossing F1 animals.

In some embodiments, the nucleic acid comprises V, D and/or J genetic sequences from at least two, three or four distinct species.

In some embodiments, the nucleic acid comprises V, D and/or J genetic sequences from at least two, three or four camelid species.

### BRIEF DESCRIPTION OF THE DRAWINGS

FIG. 1: Schematic representation of targeted integration of the Bacterial Artificial Chromosome (BAC1) construct with CH1 domain deletions (Exon 2) in mouse γ3, γ1, γ2b, and γ2a constant regions on mouse IgH locus.

FIG. 2: Schematic representation of a stepwise strategy to delete CH1 domains of γ3, γ1, γ2b, and γ2a constant regions via CRISPR-targeting in mouse ES cells.

FIG. 3: Schematic representation of a CRISPR-targeting strategy to delete CH1 domains of γ3, γ2b, and γ2a constant regions in mouse fertilized eggs.

FIG. 4: Schematic representation of genetic modifications of the IgH locus of representative transgenic lines.

FIG. 5: Western blot analysis on serum samples obtained from pre-immunized Transgenic 4 animals (TG 4) or Transgenic 6 animals (TG 6) (reducing conditions).

FIGS. 6A-6C: Western blot analysis on serum samples obtained from pre-immunized Transgenic 4 animals (TG 4) (FIG. 6A), Transgenic 6 animals (TG 6) (FIG. 6B) or Transgenic 2 animals (TG 2) (FIG. 6C) (non-reducing conditions).

FIGS. 6D-6E: Western blot analysis on serum samples obtained from pre-immunized Transgenic 4 animals (TG 4) and Transgenic 6 animals (TG 6) (FIG. 6D) or with Transgenic 2 animals (TG 2) (FIG. 6E) with light chain detection (non-reducing conditions).

FIGS. 6F-6I: ELISA quantification of IgG3 (FIG. 6F), IgG1 (FIG. 6G), IgG2b (FIG. 6H) and IgG2a (FIG. 6I) antibodies in sera samples obtained from Transgenic 2 animals (TG 2).

FIG. 7A: Graph showing antibody titres after immunization of Transgenic 6 animals with Target 1 antigens as measured by ELISA (dilution of 1/100 and 1/15000). Each line represents a distinct Transgenic 6 animal.

FIG. 7B: Western blot analysis on serum samples from Transgenic 6 animals immunized with Target 1 with an anti-IgG2a or an anti-IgG3.

FIG. 7C: Graph showing antibody titres after immunization of Transgenic 6 animals with CD3 antigens (Target 2) as measured by ELISA (dilution of 1/150 and 1/12150). Each line represents a distinct Transgenic 6 animal.

FIG. 7D: Graph showing antibody titres after immunization of Transgenic 6 animals with Target 3 antigens as measured by ELISA (dilution of 1/100 and 1/15000). Each line represents a distinct Transgenic 6 animal.

FIG. 7E: Graph showing the serum cross-reactivity from Transgenic 6 animals immunized with wild type SARS-CoV-2 spike proteins to spike glycoprotein variant B.1.351 (Beta) as measured by ELISA (day 1 and day 38 serum samples with dilutions of 1/100, 1/4000, and 1/640000).

FIG. 7F: Graph showing the serum cross-reactivity from Transgenic 6 animals immunized with SARS-CoV-2 wild type spike proteins to spike glycoprotein variant B.1.1.7 (Alpha) as measured by ELISA (day 1 and day 38 serum samples with dilutions of 1/100, 1/4000, and 1/640000).

FIG. 7G: Graph showing serum neutralization of SARS-CoV-2 wild type spike protein to the binding of human ACE2 (hACE2) target.

FIG. 7H: Graph showing antibody titres after immunization of Transgenic 2 animals with Target 3 antigens as measured by ELISA (dilution of 1/100 and 1/15000). Each line represents a distinct Transgenic 2 animal.

FIG. 7I: Graph showing the cross-reactivity of serum obtained from Transgenic 2 animals immunized with SARS-CoV-2 wild type spike proteins to spike glycoprotein variant B.1.351 (Beta) as measured by ELISA (day 1 and day 38 serum samples with dilutions of 1/100, 1/4288, and 1/643393).

FIG. 7J: Graph showing the cross-reactivity of serum obtained from Transgenic 2 animals immunized with SARS-CoV-2 wild type spike proteins to spike glycoprotein variant B.1.1.7 (Alpha) as measured by ELISA (day 1 and day 38 serum samples with dilutions of 1/100, 1/4288, and 1/643393).

FIG. 8A: Next generation sequencing (NGS) analysis comparing immune libraries derived from Transgenic 6 animals and alpacas immunized with Target 1.

FIG. 8B: Schematic illustrating the overlapping sequences in Transgenic 6 animals and alpaca immune libraries.

FIG. 9A: Graph showing binding of selected sdAb-Fcs from Transgenic 6 library to recombinant Target 1 of different species as measured by ELISA.

FIG. 9B: Graph showing binding of selected sdAb-Fcs from Transgenic 6 library to cells expressing Target 1 as measured by FACS.

FIG. 10A: Scheme illustrating treatment of NCG mice implanted with MDA-MB-453 triple-negative breast tumor cells with selected sdAb-Fcs obtained from Transgenic 6 animals immune library.

FIG. 10B: Graph showing tumor volume over time in established immuno-oncology model of MDA-MB-453 in NCG mice treated with selected anti-Target 1 sdAb-Fcs labelled sdAb1-sdAb4.

FIG. 11A: Schematic showing genomic organization of camelid V segments and surrounding camelid regulatory sequences.

FIGS. 11B and 11C: Schematic illustration of exemplary DNA constructs used for generating transgenic animals (constant region locus is not illustrated).

FIG. 12: Schematic illustration of targeted integration of exemplary Bacterial Artificial Chromosome constructs with multi-species VHH/VH gene segments and entire D and J gene segments from alpaca.

FIGS. 13A-C: Schematic representation of exemplary genetic modifications of the IgH locus of representative transgenic lines; insertion of Bactrian and alpaca VH/VHH segments and replacement of mouse D/J segments with alpaca D/J segments (FIG. 13A), insertion of Llama, Bactrian and alpaca VH/VHH segments and replacement of mouse D/J segments with alpaca D/J segments (FIG. 13B) or insertion of Bactrian, Llama and alpaca VH/VHH segments and replacement of mouse D/J segments with alpaca D/J segments (Bac4b construct) (FIG. 14C).

FIGS. 14A-B: Schematic representation of genetic modifications made on D and J gene of transgenic animals; replacement of alpaca D and J segments with Bactrian D and J segments in ES clones carrying BAC4b to generate BAC6 ES clones and transgenic animals (FIG. 14A) and insertion of Bactrian D and J segments in transgenic mice comprising of alpaca D and J segments (FIG. 14B).

FIG. 15A: Schematic representation of genetic modifications made on the ES clone carrying the BAC4a modification to generate BAC5 ES clones (constant region locus is not illustrated).

FIG. 15B: Schematic representation of genetic modifications made on the ES clone carrying the BAC5 modification to generate BAC7 ES clones (constant region locus is not illustrated).

FIG. 16A: Western blot detection using an anti-camelid VHH antibody on serum samples of BAC4b pre-immunized serum samples from chimeric animals.

FIG. 16B: Western blot detection using an anti-camelid VHH antibody on serum samples of BAC4b pre-immunized F1 heterozygous animals.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “treatment” for purposes of this disclosure refers to both therapeutic treatment and prophylactic or preventative measures. Those in need of treatment include those already with the disorder as well as those prone to have the disorder or those in whom the disorder is to be prevented.

The term “about” or “approximately” with respect to a given value means that variation in the value is contemplated. In some embodiments, the term “about” or “approximately” shall generally mean a range within +/−20 percent, within +/−10 percent, within +/−5, +/−4, +/−3, +/−2 or +/−1 percent of a given value or range.

It is to be understood herein that the term “at least” with respect to a given value intends to include the value and superior values. For example, the term “at least one” include “at least two”, “at least three”, “at least four”, “at least five”, “at least six”, “at least seven”, “at least eight”, “at least nine”, “at least ten”, “etc. For example, the term “at least 80%” include “at least 81%”, “at least 82%”, “at least 83%”, “at least 84%”, “at least 85%”, “at least 86%”, “at least 87%”, “at least 88%”, “at least 89%”, “at least 90%”, “at least 91%”, “at least 92%”, “at least 93%”, “at least 94%”, “at least 95%” “at least 96%”, “at least 97%”, “at least 98%”, “at least 99%”, “at least 99.1%”, “at least 99.2%”, at least 99.3%” at least 99.4%” at least 99.5%” at least 99.6%”, at least 99.7%” at least 99.8%”, at least 99.9%”, and 100%.

As used herein, the term “binding agent” refers to a compound that comprises an antigen-binding domain of an antibody or antigen binding fragment thereof.

As used herein the term “antigen-binding domain” refers to a domain of an antibody that is involved in binding to an antigen and includes a CDRH3, a combination of CDRH1, CDRH2, and CDRH3 or a complete variable region of an antibody or antigen binding fragment thereof.

As used herein the term “antibody” encompasses monoclonal antibody, polyclonal antibody, humanized antibody, chimeric antibody, human antibody, single domain antibody (such as VHHs, VHs, VL, nanobodies, or single domain antibodies from camelids or shark and the like), etc. The term “antibody” encompasses molecules that have a format similar to that of a naturally occurring antibody (e.g., IgGs, IgM, IgD, IgA, IgE, single domain antibody etc.) or other formats such as bispecific antibodies, minibodies, diabodies, tirabodies, tetrabodies and the like.

As used herein the term “transgene” refers to a gene or portion thereof that is introduced into the genome of a host such as a non-human animal.

As used herein the term “transgenic non-human animal” or “transgenic animal” refers to a non-human animal that carries one or more transgene(s) and encompasses chimeric animals, heterozygous animals and homozygous animals.

The terms “VH” refers to the variable region of a classical antibody heavy chain.

The term “VHH” refers to the variable region of a heavy chain only antibody.

The term VH segment refers to a V segment of a classical antibody heavy chain.

The term VHH segment refers to a V segment of a heavy chain only antibody.

It is to be understood that the term V segment as used herein refers to VH segment or to VHH segment.

The term VH polypeptide refers to the amino acid sequence encoded by a VH segment.

The term VHH polypeptide refers to the amino acid sequence encoded by a VHH segment.

The term “endogenous” with respect to a gene or segment refers to the natural gene or segment of an animal genome.

The term “endogenous V site” refers to the site or location where the V segments are located in an animal genome.

The term “endogenous D site” refers to the site or location where the D segments are located in an animal genome.

The term “endogenous J site” refers to the site or location where the J segments are located in an animal genome.

The term “non-endogenous” with respect to a gene or segment refers to a foreign gene or segment.

The term “wild type” refers to a sequence that has not been modified (i.e., non-modified or unmodified) or that occurs in nature.

The term “functional CH1 domain” refers to a CH1 domain that comprises amino acid residues that allow pairing with a light chain.

The term “deletion of the CH1 domain” refers to deletion of one or more amino acid residues of the CH1 domain that are responsible for pairing of the heavy chain with the light chain, deletion of a portion comprising such amino acid residues (i.e., referred to as partial deletion) or deletion of the whole CH1 domain (referred to as complete deletion).

The term “modification of the CH1 domain” refers to amino acid mutations or substitutions that prevents pairing of the heavy chain with the light chain.

The term “complete or partial deletion” with respect to a given gene refers to a deletion that result in the given protein or exon usually encoded by the gene not being expressed.

In the present disclosure, the genome of animals is modified so as to express single domain antibodies. Some of the transgenic animals disclosed herein may advantageously produce single domain antibodies of various genetic background and isotypes.

Generation of the transgenic animals of the present disclosure involves designing or generating nucleic acid constructs comprising the desired modifications and obtaining genetically modified embryonic stem cells or fertilized eggs. These are microinjected into a blastocyst-stage embryo and implanted into a pseudopregnant female mouse. Chimeras comprising the transgene are selected for subsequent breeding. Heterozygous or homozygous animals having the desired genetic modifications are obtained.

In the present disclosure, transgenic animals carrying a modified IgH locus are disclosed herein.

### Nucleic Acid Constructs

The nucleic acid construct disclosed herein therefore comprises sequences that allow modification of an IgH locus of an animal.

In exemplary embodiments, DNA constructs are designed to allow modification of an IgH locus in mice or mice embryonic stem cells.

The DNA construct comprises sequences for homologous recombination and usually antibiotic resistance genes or markers allowing selection of cells that have incorporated the transgene. For example, the DNA construct may comprise loxP sites and homology arms so that the gene(s) of interest are inserted at a desired location within the genome of the animal such as for example at a mice IgH locus.

In some instances, the DNA construct is co-injected with CRISPR construct targeting the inner homologous sequence to enhance the incorporation.

In other instances, the DNA construct comprises sequences for gene targeting with gene-editing tools such as the clustered regularly interspaced palindromic repeats (CRISPR)-Cas9 system, zinc finger nucleases (ZFNs) system, transcription activator-like effector nucleases (TALENS) system or the like.

The DNA construct of the present disclosure comprises for example a gene having a deletion or modification of the CH1 domain of an endogenous mouse γ3 gene, γ1 gene, γ2b gene and/or or γ2a gene.

Deletion of the CH1 domain includes partial deletion or complete deletion of the CH1 domain. Complete deletion of the CH1 domain is particularly contemplated.

Modifications of the CH1 domain include mutations in the amino acid residues involved in pairing with a light chain resulting in a significant decrease or absence or pairing. Other modifications in the CH1 domain include nucleic acid mutations that result in the CH1 exon not being incorporated into the messenger RNA.

Alternatively, the DNA construct comprises a gene having a deletion or modification of the CH1 domain of at least one endogenous mouse gene selected from γ3 gene, γ1 gene, γ2b gene and/or or γ2a gene in combination with a complete or partial deletion of at least one endogenous mouse gene selected from γ3 gene, γ1 gene, γ2b gene and/or or γ2a gene.

In some instances, the DNA construct can also comprise V, D and/or J gene segments such as unrearranged V, D and/or J gene segments and associated introns comprising recombination signal sequences for VDJ rearrangement.

The DNA construct can comprise multiple D and/or J gene segments. In some instances, the multiple D and/or J gene segments all originate from a single species. In other instances, the multiple D and/or J gene segments originate from at least two species. In yet other instances, the multiple D and/or J gene segments originate from at least three species (including 4 and 5 species).

The DNA construct can comprise multiple V gene segments. In some instances, the multiple V gene segments all originate from a single species. In other instances, the multiple V gene segments originate from at least two species. In yet other instances, the multiple V gene segments originate from at least three species. In other instances, the multiple V gene segments originate from at least four species. In further instances, the multiple V gene segments originate from at least five species.

The DNA construct can thus comprise one or more unrearranged camelid D and/or J gene segments and a gene comprising a partial or complete deletion or modification of the CH1 domain of at least one IgG constant region gene.

Alternatively, the DNA construct can comprise one or more unrearranged camelid V, D and/or J gene segments and a gene comprising a partial or complete deletion or modification of the CH1 domain of at least one IgG constant region gene.

In some instances, the modified IgG constant region gene included in the DNA constructs is a modified mouse IgG constant region gene.

In other instances, the modified IgG constant region gene included in the DNA constructs is a modified human IgG constant region gene.

The DNA construct can comprise any of the modified immunoglobulin gamma genes disclosed herein.

The DNA construct can comprise any of the V, D and J segments combination disclosed herein.

DNA constructs that are currently used in genetic manipulations include artificial chromosomes such as for example and without limitation, bacterial artificial chromosomes, yeast artificial chromosomes, or mammalian artificial chromosomes.

A sequence integrated within an animal's genome may be identified as a transgene.

### V, D and J Segments and Transgenes

As disclosed herein, the transgenic non-human animal comprises unrearranged V, D and/or J gene segments from camelids or from another mammal such as for example, a human or a rodent or a combination thereof.

In some embodiments, the transgenic non-human animal comprises genomic V, D and/or J gene segments from camelids.

The transgenic non-human animal thus comprises genomic camelid V, D and/or J gene segments that includes original camelid regulatory sequences associated with each V, D and/or J segments.

For examples, each camelid V segment includes approximately 5 kb upstream and approximately 5 kb downstream of the V segment and includes camelid regulatory sequences, camelid intronic sequences, camelid leader sequences and camelid recombination signal sequences surrounding the V segment.

Therefore, the unrearranged camelid V segments include surrounding camelid regulatory regions, surrounding camelid intronic sequences, surrounding camelid leader sequences and surrounding camelid RSS.

In some embodiment, the unrearranged camelid D segments include surrounding camelid regulatory regions, camelid intronic sequences, camelid leader sequences and camelid RSS.

For examples, each camelid D segment includes camelid regulatory sequences, camelid intronic sequences, camelid leader sequences and camelid recombination signal sequences surrounding the D segment.

In some embodiment, the unrearranged camelid J segments include surrounding camelid regulatory regions, camelid intronic sequences, camelid leader sequences and camelid RSS.

For examples, each camelid J segment includes camelid regulatory sequences, camelid intronic sequences, camelid leader sequences and camelid recombination signal sequences surrounding the J segment.

The camelid regulatory sequences, camelid intronic sequences, camelid leader sequences and/or camelid recombination signal sequences of the DNA construct, transgene or transgenic non-human animal therefore correspond to the genomic camelid regulatory sequences, genomic camelid intronic sequences, genomic camelid leader sequences and/or genomic camelid recombination signal sequences.

The unrearranged camelid V gene segments therefore comprises associated introns comprising recombination signal sequences for VDJ rearrangement.

Each camelid V gene segments comprises its originals regulatory sequences.

The transgene may be introduced within an animal's genome by knock-out/knock-in technology at the IgH locus.

The V segment of the heavy chain encodes a major portion of an antibody variable region including framework 1 (FR1), CDRH1, framework 2 (FR2), CDRH2, framework 3 (FR3) and a portion of the CDR3.

The D and J segment encodes the rest of CDR3 while the J segment also encode framework four (4).

Some of the transgenic non-human animals of the present disclosure carry a transgene comprising camelid D and/or J segments. In some instances, all camelid D and/or J segments are from one camelid species. In other instances, the camelid D and/or J segments are from multiple camelid species. In exemplary embodiments, all endogenous D and/or J segments are replaced for camelid D and/or J segments. In other exemplary embodiments, some endogenous D and/or J segments are preserved, and camelid D and/or J segments are inserted. In yet other exemplary embodiments, all endogenous D and/or J segments are preserved, and camelid D and/or J segments are inserted.

Some of the transgenic non-human animals of the present disclosure may carry a combination of V gene segments from multiple species. For example, the transgenic non-human animal may comprise endogenous V gene segments in addition to foreign V gene segments. The transgenic mice of the present disclosure comprise for example, mice V segments as well as camelid V segments. However, any combination of V segments from rodents, camelids or human is also encompassed herein.

Some of the transgenic non-human animals of the present disclosure carry a transgene comprising camelid V gene segments. In some instances, the V gene segments are all from one camelid species. In other instances, the V gene segments are from at least two camelid species. In yet other instances, the V gene segments are from at least three camelid species. In additional instances, the V gene segments are from at least four camelid species. In additional instances, the V gene segments are from at least five camelid species.

In some exemplary embodiments, the transgenic non-human animal comprises a transgene comprising V, D and J gene segments from a camelid. In yet other exemplary embodiments, the transgene comprises V, D and J gene segments from a human. In additional exemplary embodiments, the transgene comprises V, D and J gene segments from a rodent.

In exemplary embodiments, the V segments are from a rodent and the D and J segments are from a camelid. In exemplary embodiments, the V segments are from a rodent and the D and J segments are from a rodent and from a camelid. In exemplary embodiments, the V segments are from a rodent and from a camelid and the D and J segments are from a rodent and from a camelid.

In exemplary embodiments, the V D and/or J gene segments are selected from alpacas, from Bactrians, from llamas, Vicunia and/or from dromedaries or combination thereof.

In an exemplary embodiment, the V, D and J segments are combined in such a manner that the DNA construct or transgene comprises in 5′ to 3′ fashion mouse VH segments, alpaca VH and/or VHH segments, alpaca D segments and alpaca J segments.

In another exemplary embodiment, the V, D and J segments are combined in such a manner that the DNA construct or transgene comprises in 5′ to 3′ fashion mouse VH segments, Bactrian VH and/or VHH segments, alpaca VH and/or VHH segments, alpaca D segments and alpaca J segments.

In another exemplary embodiment, the V, D and J segments are combined in such a manner that the DNA construct or transgene comprises in 5′ to 3′ fashion mouse VH segments, llama VH and/or VHH segments, alpaca VH and/or VHH segments, alpaca D segments and alpaca J segments.

In yet another exemplary embodiment, the V, D and J segments are combined in such a manner that the DNA construct or transgene comprises in 5′ to 3′ fashion mouse VH segments, llama VH and/or VHH segments, Bactrian VH and/or VHH segments, alpaca VH and/or VHH segments, alpaca D segments and alpaca J segments.

In a further exemplary embodiment, the V, D and J segments are combined in such a manner that the DNA construct or transgene comprises in 5′ to 3′ fashion mouse VH segments, Bactrian VH and/or VHH segments, llama VH and/or VHH segments, alpaca VH and/or VHH segments, alpaca D segments and alpaca J segments.

In an additional exemplary embodiment, the V, D and J segments are combined in such a manner that the DNA construct or transgene comprises in 5′ to 3′ fashion mouse VH segments, llama VH and/or VHH segments, Bactrian VH and/or VHH segments, alpaca VH and/or VHH segments, alpaca D segments, Bactrian D segments, Bactrian J segments, and alpaca J segments.

In an exemplary embodiment, the V, D and J segments are combined in such a manner that the DNA construct or transgene comprises in 5′ to 3′ fashion mouse VH segments, llama VH and/or VHH segments, Bactrian VH and/or VHH segments, alpaca VH and/or VHH segments, Bactrian D segments and Bactrian J segments.

In an exemplary embodiment, the V, D and J segments are combined in such a manner that the DNA construct or transgene comprises in 5′ to 3′ fashion mouse VH segments, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

In an exemplary embodiment, the V, D and J segments are combined in such a manner that the DNA construct or transgene comprises in 5′ to 3′ fashion, alpaca VH and/or VHH segments, Bactrian VH and/or VHH segments, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

The DNA construct or transgene can comprise for example, from one to at least seven D gene segments of alpacas.

Alternatively, the DNA construct or transgene can comprise from one to at least seven J gene segments of alpacas.

In a further exemplary embodiment, the DNA construct or transgene may comprise from one to at least seven (e.g., 1, 2, 3, 4, 5, 6, 7 or more than 7) Bactrian D gene segments.

In yet a further exemplary embodiment, the DNA construct or transgene may comprise from one to at least seven (e.g., 1, 2, 3, 4, 5, 6, 7 or more than 7) Bactrian J gene segments.

In a further exemplary embodiment, the DNA construct or transgene may comprise from one to at least seven (e.g., 1, 2, 3, 4, 5, 6, 7 or more than 7) dromedaries D gene segments.

In yet a further exemplary embodiment, the DNA construct or transgene may comprise from one to at least seven (e.g., 1, 2, 3, 4, 5, 6, 7 or more than 7) dromedaries J gene segments.

In a further exemplary embodiment, the DNA construct or transgene may comprise from one to at least seven (e.g., 1, 2, 3, 4, 5, 6, 7 or more than 7) llama D gene segments.

In yet a further exemplary embodiment, the DNA construct or transgene may comprise from one to at least seven (e.g., 1, 2, 3, 4, 5, 6, 7 or more than 7) llama J gene segments.

In a further exemplary embodiment, the DNA construct or transgene may comprise from one to at least seven (e.g., 1, 2, 3, 4, 5, 6, 7 or more than 7) Vicunia D gene segments.

In yet a further exemplary embodiment, the DNA construct or transgene may comprise from one to at least seven (e.g., 1, 2, 3, 4, 5, 6, 7 or more than 7) Vicunia J gene segments.

In another exemplary embodiment, the DNA construct or transgene may comprise from one to at least six (e.g., 1, 2, 3, 4, 5, 6 or more than 6) alpaca V gene segments.

In another exemplary embodiment, the DNA construct or transgene may comprise all V gene segments of an alpaca.

In yet another exemplary embodiment, the DNA construct or transgene may comprise from one to at least ten (e.g., 1, 2, 3, 4, 5, 6, 7, 8, 9, 10 or more than 10) Bactrians V gene segments.

In another exemplary embodiment, the DNA construct or transgene may comprise all V gene segments of a Bactrian.

In a further exemplary embodiment, the DNA construct or transgene may comprise from one to at least ten (e.g., 1, 2, 3, 4, 5, 6, 7, 8, 9, 10 or more than 10) llama V gene segments.

In another exemplary embodiment, the DNA construct or transgene may comprise all V gene segments of a llama.

In a further exemplary embodiment, the DNA construct or transgene may comprise from one to at least six (e.g., 1, 2, 3, 4, 5, 6 or more than 6) dromedary V gene segments.

In another exemplary embodiment, the DNA construct or transgene may comprise all V gene segments of a dromedary.

In a further exemplary embodiment, the DNA construct or transgene may comprise from one to at least six (e.g., 1, 2, 3, 4, 5, 6 or more than 6) Vicunia V gene segments.

In another exemplary embodiment, the DNA construct or transgene may comprise all V gene segments of a Vicunia.

Exemplary and non-limiting embodiments of camelid VH/VHH, D and J polypeptides encoded by V, D and/or J segments are provided herein.

In some embodiments, the DNA construct, transgene or transgenic animal comprises V, D and J segments as outlined in Table 1 (mouse VH, D or J may also be included). However, additional configurations are possible. The order and number of V segments in Table 1 may vary. The order and number of D segments in Table 1 may vary. The order and number of J segments in Table 1 may vary.

TABLE 1

| V segments | D segments | J segments |
|---|---|---|
| One alpaca VH | Seven alpaca D segments | Seven alpaca J segments |
| One alpaca VHH | | |
| Three Bactrian VHs | Seven alpaca D segments | Seven alpaca J segments |
| Three Bactrian VHHs | | |
| One alpaca VH | | |
| One alpaca VHH | | |
| Two llama VHs | Seven alpaca D segments | Seven alpaca J segments |
| Three llama VHHs | | |
| One alpaca VH | | |
| One alpaca VHH | | |
| Two llama VHs | Seven alpaca D segments | Seven alpaca J segments |
| Three llama VHHs | | |
| Three Bactrian VHs | | |
| Three Bactrian VHHs | | |
| One alpaca VH | | |
| One alpaca VHH | | |
| Two Bactrian VHs | Seven alpaca D segments | Seven alpaca J segments |
| Two Bactrian VHHs | | |
| Two llama VHs | | |
| Three llama VHHs | | |
| One alpaca VH | | |
| One alpaca VHH | | |
| Two llama VHs | Seven Bactrian D segments | Seven Bactrian J segments |
| Three llama VHHs | | |
| Three Bactrian VHs | | |
| Three Bactrian VHHs | | |
| One alpaca VH | | |
| One alpaca VHH | | |
| Two llama VHs | Seven alpaca D segments | Seven Bactrian J segments |
| Three llama VHHs | Seven Bactrian D segments | Seven alpaca J segments |
| Three Bactrian VHs | | |
| Three Bactrian VHHs | | |
| One alpaca VH | | |
| One alpaca VHH | | |
| One llama VHs | Seven alpaca D segments | Seven alpaca J segments |
| Two llama VHHs | | |
| One dromedary VH | | |
| Three dromedary VHHs | | |
| Two alpaca VHs | | |
| Two alpaca VHHs | | |
| Three Bactrian VHs | Seven alpaca D segments | Seven alpaca J segments |
| Three Bactrian VHHs | | |
| Two alpaca VHs | | |
| Two alpaca VHHs | | |
| Three Bactrian VHHs | Seven alpaca D segments | Seven alpaca J segments |
| Two Bactrian VHs | | |
| Two llama VHHs | | |
| Two llama VHs | | |
| One alpaca VHH | | |
| One alpaca VH | | |
| Three Bactrian VHHs | Seven alpaca D segments | Seven alpaca J segments |
| Three Bactrian VHs | | |
| Four llama VHHs | | |
| Three llama VHs | | |
| Three alpaca VHHs | | |
| Three alpaca VHs | | |
| Three dromedary VHHs | | |
| One dromedary VH | | |
| Three Bactrian VHHs | Seven Bactrian D segments | Seven Bactrian J segments |
| Two Bactrian VHs | | |
| Two llama VHHs | | |
| Two llama VHs | | |
| One alpaca VHH | | |
| One alpaca VH | | |
| Six Bactrian VHHs | Seven alpaca D segments | Seven alpaca J segments |
| Six Bactrian VHs | | |
| Four llama VHHs | | |
| Three llama VHs | | |
| Five alpaca VHHs | | |
| Five alpaca VHs | | |
| Three dromedary VHHs | | |
| One dromedary VH | | |

In some embodiments, the camelid V, D and/or J segments may be modified especially in the framework regions. Modifications of the framework regions include replacing camelid framework regions with sequences that are at least 80% identical to a naturally occurring sequence. Other modifications include replacing camelid framework regions for frameworks that are from about 80% to about 100% (e.g., about 80%, 85%, 90%, 95%, 99%, or 100%) identical to human framework regions so as to produce humanized HCAbs having camelid CDRs.

### Embryonic Stem Cells

Embryonic stem cells are selected based on the desired animal species and desired genetic background.

Genetically modified embryonic stem cells are obtained by electroporation of a DNA construct comprising the modified gene(s) as well as sequences for homologous recombination and selection.

In some embodiments, the embryonic stem cell is an isolated embryonic non-human stem cell comprising a germline modifications at an immunoglobulin heavy chain (IgH) locus which comprises a) replacement of one or more of the endogenous mouse V gene segments for one or more unrearranged camelid V gene segments or insertion of unrearranged camelid V gene segments, b) replacement of at least one or all of the endogenous mouse D and J segments with camelid D and J segments and c) deletion or modification of the CH1 domain of at least one or all of endogenous mouse γ1, γ2a, γ2b and γ3 gene so that a polypeptide expressed from said endogenous mouse γ1, γ2a, γ2b and γ3 gene does not comprise a functional CH1 domain.

In some embodiments, the embryonic stem cell is an isolated embryonic non-human stem cell comprising a germline modifications at an immunoglobulin heavy chain (IgH) locus which comprises deletion of the CH1 domain of each of the endogenous γ3 gene, γ1 gene, γ2b gene and γ2a gene, replacement of mouse D and J gene segments for unrearranged camelid D and J gene segments, insertion of camelid V gene segments from multiple camelid species and optionally deletion of at least one or all endogenous mouse V gene segments.

Alternatively, embryonic stem cells may be genetically modified with gene-editing tools such as the clustered regularly interspaced palindromic repeats (CRISPR)-Cas9 system, zinc finger nucleases (ZFNs) system, transcription activator-like effector nucleases (TALENS) system or the like.

The presence of the transgene in the ES cell genome is confirmed by sequencing and ES cells carrying the correct sequence are amplified for subsequent use. Quality control tests such as karyotyping are also usually performed.

Embryonic stem cells may be totipotent, multipotent or pluripotent. However, totipotent embryonic stem cells are usually used for generating a transgenic non-human animal.

Embryonic stem cells comprising the genetic modifications described herein are encompassed by the present disclosure.

Embryonic stem cells may be derived from any of the transgenic animals disclosed herein.

Embryonic stem cells that are already genetically modified (whether by homologous recombination or isolated from transgenic animals) may be used to make further genetic modifications as needed.

### Transgenic Non-Human Animals

Transgenic non-human animals of the present disclosure include small animals that are amenable to genetic manipulation. However large animals such as cows, sheep and the like may also be suitable. Accordingly, in some embodiments, a method of making a transgenic non-human animal is provided, comprising the use of any one or more of the nucleic acid constructs disclosed herein.

For the purpose of the present application, rodents such as rats and mice are particularly selected. However, other small animals may be suitable such as rabbits or chickens.

The selection of small animal for expression of antibodies is associated with several advantages. For example, a small amount of antigen is sufficient to generate an immune response and a small amount of blood from immunized animals may be sufficient to represent the full antibody repertoire. In addition, modification of the genome to include V, D and/or J segments from multiple camelid species as disclosed herein increases the diversity of single domain antibodies produced. Moreover, they are characterized by a short reproduction cycle.

Finally, producing single domain antibodies comprising sequences from multiple camelid species in transgenic animals is more advantageous than producing them in camelids since generating the same diversity of antibodies would require separate immunization for each camelid species.

Expressing single domain antibodies in mice of various genetic background as disclosed herein is also expected to increase the diversity.

Various methods for obtaining a transgenic non-human animal are available.

One of such method involves the use of genetically modified embryonic stem cells. Another method involves the use of genetically modified fertilized eggs.

Genetically modified embryonic stem cells or fertilized eggs are microinjected into a blastocyst-stage embryo which is then implanted into a pseudopregnant female mouse. Chimeras comprising the transgene in their germ cells are selected for subsequent breeding.

The transgenic non-human animals of the present disclosure comprise germline modifications at an immunoglobulin heavy chain (IgH) locus.

In some embodiments, transgenic non-human animals are generated with all modifications on the same allele. In some embodiments, transgenic non-human animals are generated with modifications on both alleles. In some embodiments, both alleles may be the same. In other embodiments, both alleles are different.

The modifications include for example a deletion or modification of the CH1 domain of an endogenous immunoglobulin gamma gene. Other modifications include for example a deletion or modification of the CH1 domain of an endogenous immunoglobulin gamma gene in combination with partial or complete deletion or modification of at least one other endogenous immunoglobulin gamma gene.

The modifications include for example a deletion or modification of the CH1 domain of an endogenous non-human animal γ3 gene, γ1 gene, γ2b gene and/or or γ2a gene.

Other modifications include deletion or modification of the CH1 domain of at least one endogenous non-human animal gene selected from γ3 gene, γ1 gene, γ2b gene and/or or γ2a gene in combination with a complete or partial deletion of at least one endogenous non-human animal gene selected from γ3 gene, γ1 gene, γ2b gene and/or or γ2a gene.

In some instances, modifications in the endogenous immunoglobulin gamma gene are also accompanied with modification in the variable region.

For example, some modifications include a) replacement of one or more endogenous non-human D and/or J gene segments for one or more unrearranged camelid D and/or J gene segments and b) partial or complete deletion or modification of the CH1 domain of at least one IgG constant region gene. Other modifications include for example, a) insertion of one or more unrearranged camelid D and/or J gene segments at an IgH locus and b) partial or complete deletion or modification of the CH1 domain of at least one IgG constant region gene.

Other modifications include a) replacement of one or more endogenous non-human V, D and/or J gene segments for one or more unrearranged camelid V, D and/or J gene segments and b) partial or complete deletion or modification of the CH1 domain of at least one IgG constant region gene.

Again, these modifications may also be combined with partial or complete deletion of at least one endogenous immunoglobulin gamma gene.

Modifications in the IgH locus may be present in both alleles such as in the case of homozygous animals. As such, both allele of the transgenic non-human animal genome may comprise an identical IgH locus.

Alternatively, modifications in the IgH locus may be present in a single allele such as in the case of heterozygous animals.

In some instances, one allele of the transgenic non-human animal genome may comprise a modified IgH locus and the other allele may be wild type.

In other instances, both allele of the transgenic non-human animal genome may comprise identical constant region genes and different V, D and/or J segments.

In yet other instances, both allele of the transgenic non-human animal genome may comprise different constant region genes and identical V, D and/or J segments.

In yet other instances, both allele of the transgenic non-human animal genome may comprise different constant region genes and different segment amongst V, D and/or J segments.

Transgenic non-human animals carrying any of the modification in the constant region disclosed herein and/or carrying V, D and/or J segments or transgenes disclosed herein are encompassed by the present disclosure.

In exemplary embodiments, transgenic non-human animals of the present disclosure comprise a germline modification at an IgH locus selected from the group consisting of:
a. deletion of the CH1 domain of an endogenous mouse γ3 gene, γ1 gene, γ2b gene and/or or γ2a gene, or;
b. deletion of the CH1 domain of at least one endogenous mouse gene selected from γ3 gene, γ1 gene, γ2b gene and/or or γ2a gene in combination with a complete or partial deletion of at least one endogenous mouse gene selected from γ3 gene, yl gene, γ2b gene and/or or γ2a gene.

In another exemplary embodiments, transgenic non-human animals of the present disclosure comprise a germline modification at an IgH locus selected from the group consisting of:
a. modification of the CH1 domain of an endogenous mouse γ3 gene, γ1 gene, γ2b gene and/or or γ2a gene, or;
b. modification of the CH1 domain of at least one endogenous mouse gene selected from γ3 gene, γ1 gene, γ2b gene and/or or γ2a gene in combination with a complete or partial deletion of at least one endogenous mouse gene selected from γ3 gene, yl gene, γ2b gene and/or or γ2a gene.

It is to be understood herein that any deletion or modification of the CH1 domain are encompassed by the present disclosure so long as the CH1 domain is deleted from the final polypeptide sequence or so long that the deletion or modification results in a heavy chain that is not able to pair with a light chain.

Some of the transgenic non-human animals of the present disclosure may be modified to express heavy chain variable regions from various species, including for example, camelid VH/VHH, D and/or J polypeptides or human VH, D and/or J polypeptides or combination thereof.

Some of the transgenic non-human animals of the present disclosure may be modified to express modified heavy chain variable regions, including for example, non-naturally occurring or modified camelid VH/VHH, D and/or J polypeptides, non-naturally occurring or modified human VH, D and/or J polypeptides or non-naturally occurring or modified VH, D and/or J polypeptides from rodents.

In some exemplary embodiments, transgenic non-human animals of the present disclosure comprise a) unrearranged heavy chain variable (V), diversity (D) and joining (J) gene segments and where the D and/or J gene segments comprise camelid D and/or J gene segments and b) at least one IgG constant region gene lacking a functional CH1 domain.

In yet other exemplary embodiments, transgenic non-human animals of the present disclosure comprise a) unrearranged heavy chain variable (V), diversity (D) and joining (J) gene segments and where the V, D and/or J gene segments comprise camelid V, D and/or J gene segments and b) at least one IgG constant region gene lacking a functional CH1 domain.

In some embodiments the IgG constant region gene of the transgenic non-human animals comprises an endogenous IgG constant region gene.

In some embodiments, the transgenic non-human animals of the present disclosure comprise:
a. a γ3 constant region gene comprising a partial or complete deletion in the region encoding the CH1 domain;
b. a γ1 constant region gene comprising a partial or complete deletion in the region encoding the CH1 domain;
c. a γ2b constant region gene comprising a partial or complete deletion in the region encoding the CH1 domain;
d. a γ2a constant region gene comprising a partial or complete deletion in the region encoding the CH1 domain or;
e. combination thereof.

In some embodiments, the transgenic non-human animals of the present disclosure comprise a partial or complete deletion in the region encoding the CH1 domain of at least two of the γ3, γ1, γ2b, γ2a constant region gene.

In some embodiments, the transgenic non-human animals of the present disclosure comprise a partial or complete deletion in the region encoding the CH1 domain of at least three of the γ3, γ1, γ2b, γ2a constant region gene.

In some embodiments, the transgenic non-human animals of the present disclosure comprise a partial or complete deletion in the region encoding the CH1 domain of each of the γ3, yl, γ2b, γ2a constant region gene.

In some embodiments, the endogenous D and J segments of the transgenic non-human animals are replaced with unrearranged camelid D and J segments. In other embodiments, camelid D and J segments can be inserted without deleting the endogenous D and J segments resulting in at least some or all of the endogenous D and J segments being preserved.

In some embodiments the transgenic non-human animals comprise unrearranged D and J segments from alpacas.

In other embodiments, the transgenic non-human animals comprise unrearranged D and J segments from Bactrians.

In some embodiments, the transgenic non-human animals comprise unrearranged D and J segments from llamas.

In other embodiments, the transgenic non-human animals comprise unrearranged D and J segments from dromedaries.

In other embodiments, the transgenic non-human animals comprise unrearranged D and J segments from Vicunias.

In other embodiments, the endogenous V segments are replaced with unrearranged camelid V segments. However, unrearranged camelid V segments can be inserted without deleting the endogenous V segments resulting in at least some or all of the endogenous V segments being preserved.

In some embodiments, the unrearranged V segments encode one or more VH and/or VHH polypeptide from an alpaca.

In some embodiments, the unrearranged V segments encode one or more VH and/or VHH polypeptide from Bactrians.

In some embodiments, the unrearranged V segments encode one or more VH and/or VHH polypeptide from a llama.

In some embodiments, the unrearranged V segments encode one or more VH and/or VHH polypeptide from dromedaries.

In some embodiments, the unrearranged V segments encode one or more VH and/or VHH polypeptide from Vicunias.

In some embodiments, the transgenic non-human animals comprise unrearranged V, D and/or J segments from multiple camelid species including for example and without limitations, from alpacas, llamas, Bactrians, Vicunias or dromedaries.

In some embodiments, the transgenic non-human animals comprise unrearranged D and J segments from alpaca and from Bactrians.

In some embodiments, the transgenic non-human animals comprise unrearranged alpaca VH and/or VHH segments, alpaca D segments and alpaca J segments.

In some embodiments, the transgenic non-human animals comprise unrearranged Bactrian VH and/or VHH segments, alpaca VH and/or VHH segments, alpaca D segments and alpaca J segments.

In some embodiments, the transgenic non-human animals comprise unrearranged llama VH and/or VHH segments, alpaca VH and/or VHH segments, alpaca D segments and alpaca J segments.

In some embodiments, the transgenic non-human animals comprise unrearranged llama VH and/or VHH segments, Bactrian VH and/or VHH segments, alpaca VH and/or VHH segments, alpaca D segments and alpaca J segments.

In some embodiments, the transgenic non-human animals comprise unrearranged Bactrian VH and/or VHH segments, llama VH and/or VHH segments, alpaca VH and/or VHH segments, alpaca D segments and alpaca J segments.

In some embodiments, the transgenic non-human animals comprise unrearranged llama VH and/or VHH segments, Bactrian VH and/or VHH segments, alpaca VH and/or VHH segments, alpaca D segments, Bactrian D segments, Bactrian J segments, and alpaca J segments.

In some embodiments, the transgenic non-human animals comprise unrearranged llama VH and/or VHH segments, Bactrian VH and/or VHH segments, alpaca VH and/or VHH segments, Bactrian D segments and Bactrian J segments.

In some embodiments, the transgenic non-human animals comprise unrearranged V segments encoding one or more mouse VH polypeptide.

In some embodiments, the transgenic non-human animals comprise unrearranged V segments encoding one or more human VH polypeptide.

The transgenic non-human animal may also carry additional genetic modifications. For example, the transgenic non-human animal may comprise partial or complete deletion of the immunoglobulin kappa and/or immunoglobulin lambda locus.

Also additional V, D and/or J segments may be inserted by further genetically modifying the transgenic non-human animal disclosed herein.

### Heavy Chain Only Antibodies (HCAbs)

In some aspects, transgenic animals of the present disclosure are immunized with an antigen of interest using standard immunization protocols.

A blood sample from the immunized transgenic animal is collected and the amino acid or nucleic acid sequence of one or of a plurality of antibody species is determined.

In some instances, the sequence of one or more complementarity determining regions is obtained. For example, the sequence of the CDRH3 region is determined. In yet other instances, the sequence of the CDRH1, CDRH2 and CDRH3 region is obtained. In yet other instances, the sequence of the entire variable region is obtained. In other instances, the sequence of the entire antibody is obtained.

Using computer-based technology, the sequence is organized in clusters. The biological function (e.g., binding, specificity, affinity, effectiveness or else) of a representative antibody species, of a fraction of the antibody pool or of the entire antibody pool within the cluster is determined. In some instances, a computer-based prediction model of the biological function is established.

The sequence information of one or more selected single domain antibody is used to make binding agents such as for example and without limitations, an antibody (including bi-, tri-, multi-specific antibody), a single domain antibody, a single chain Fv, a chimeric antigen receptor (CAR), a bispecific T cell engager (BiTE), a bispecific killer cell engager (BiKE), a trispecific killer cell engager (TriKE), a binding agent having the format as disclosed in U.S. Provisional appl. No. 62/951,701 (the entire content of which is incorporated herein by reference), or an antigen binding fragment thereof.

The present disclosure therefore provides in other aspects and embodiments, binding agents that comprise an amino acid sequence obtained from at least one single domain antibody generated by the transgenic non-human animal of the present disclosure.

Further embodiments, features, and advantages, as well as the structure and operation of the various embodiments, are described in detail below with reference to accompanying drawings.

### EXAMPLES

### Example 1—Preparation of DNA Constructs for Targeted Modification of Mouse IgH Locus

BAC1 is an engineered bacterial artificial chromosome (BAC) construct that contains entire constant regions of mouse γ3, γ1, γ2b, and γ2a with deletions of the CH1 domain (exon2 for all four subclasses). The BAC1 construct is 92 kb in length and comprises in a 5′ to 3′ fashion, the γ3 constant region in which exon 2 (CH1 domain) is replaced with a Neomycin/Kanamycin resistance gene cassette flanked by 2 loxP sites, followed by CH1-deleted γ1 and γ2b genes, and the γ2a constant region in which exon 2 (CH1 domain) is replaced with an hygromycin resistance gene cassette flanked by 2 loxP5171 sites.

BAC2 is an engineered bacterial artificial chromosome construct that contains Alpaca (Vicugna pacos) VHH3-1 (IMGT gene, IGHV3-3), and VH3-1 (IMGT gene, IGHV3-1) variable heavy chain gene segments, the entire Alpaca IGHD gene segments (IMGT ID: IGHD1-IGHD8) and Alpaca IGHJ gene segments (IMGT ID: IGHJ1-IGHJ7). The BAC2 construct is 100 kb in length and include in a 5′ to 3′ fashion a 5 kb arm of mouse homologous sequence targeting mouse genomic IGHV5-1 and IGHV2-1 genes, followed by a Neomycin/Kanamycin resistance gene cassette flanked by two FRT sites, the Alpaca genomic DNA fragment insert, the hygromycin resistance gene cassette flanked by two FRT-F3 sites and a 5 kb arm of mouse homologous sequences.

BAC3a is a multi-species construct that includes Alpaca and Bactrian VHH and VH gene segments. BAC3a is a 147 kb construct that is based on the BAC2 construct modified by inserting a 47 kb Bactrian DNA fragment between the Neomycin/Kanamycin resistance gene cassette and the Alpaca genomic DNA fragment. The Bactrian DNA fragment contains three VHH gene segments (BctVhh_*1, BctVhh_*2, BctVhh_*3) and three VH gene segments (BctVh_*1, BctVh_*2, BctVh_*3).

BAC3b is a multi-species construct that includes Alpaca and llama VHH and VH gene segments. BAC3b is a 160 kb construct that is based on the BAC2 construct modified by inserting a 60 kb llama DNA fragment between the Neomycin/Kanamycin resistance gene cassette and the Alpaca genomic DNA fragment. The llama DNA fragment contains two VHH gene segments (LmVhh3_*3 and LmVhh3_*4) and two VH gene segments (lmVh_*1, lmVh_*2).

BAC4a is a multi-species construct that includes Alpaca, Bactrian, and Llama VHH and VH gene segments. The BAC4a construct is 196 kb that is based on the BAC3a construct modified by inserting a 49 kb Llama DNA fragment between the Neomycin/Kanamycin resistance gene cassette and the Bactrian DNA fragment. The Llama DNA fragment contains two VHH gene segments (LmVhh3_*3 and LmVhh3_*4) and two VH gene segments (LmVh_*1 and LmVh_*2).

BAC4b is a multi-species construct that includes Alpaca, Bactrian, and Llama VHH and VH gene segments. The BAC4b construct is 212 kb that is based on the BAC3b construct modified by inserting a 52 kb Bactrian DNA fragment between the Neomycin/Kanamycin resistance gene cassette and the llama DNA fragment. The Bactrian DNA fragment contains three VHH gene segments (BacVhh3_*10, BacVhh3_*11 and BacVhh3_*12) and two VH gene segments (BacVh_*4 and BacVh_*5).

BAC5 is a multi-species construct that includes llama, dromedary, and alpaca VHH and VH gene segments. The BAC5 construct is 148 kb in length targeting 5′ upstream to the Bac4a construct in the identified Bac4a positive ES cell clone to introduce additional VHH and VH genes. The 42 kb llama DNA fragment contains two VHH gene segments (LmVhh3_*1, and LmVhh3_*2) and one VH gene segments (LmVh_*3). The 54 kb dromedary DNA fragment contains three VHH gene segments (DmdVhh3_*5, DmdVhh3_*6 and DmdVhh3_*7) and one VH gene segment (DmdVh_*1). The 33 kb alpaca DNA fragment contains two VHH gene segments (alVhh3_*1 and alVhh3_*2) and two VH gene segments (alVh_*1 and alVh_*2).

BAC6 is an engineered BAC construct that contains the DNA fragment including the entire Bactrian IGHD gene segments and Bactrian IGHJ gene segments based on sequence alignment analysis to known alpaca and dromedary genomic sequences. The BAC6 construct is 59 kb in length and include in a 5′ to 3′ fashion a 3 kb arm of a 5′ alpaca homologous sequence targeting upstream of alpaca D/J gene segments, followed by a hygromycin resistance gene cassette flanked by two Loxp511 sites, the Bactrian genomic DNA fragment insert, the 3 kb arm of mouse homologous sequences.

BAC7 is a multi-species construct that includes alpaca and Bactrian VHH and VH gene segments. The BAC7 construct is 129 kb in length targeting 5′ upstream of the Bac5 construct in the identified BAC5 positive ES cell clone to introduce additional VHH and VH genes. The 48 kb alpaca DNA fragment contains two VHH gene segments (alVhh3_*3, and alVhh3_*5) and two VH gene segments (alVh_*3, alVh_*4). The 72 kb Bactrian DNA fragment contains three VHH gene segments (BacVhh3_*9, BacVhh3_*4 and BacVhh3_*5) and three VH gene segments (BacVh_*6, BacVh_*7, and BacVh_*8).

### Example 2—Generation of Genetically Modified ES Cells and Fertilized Eggs

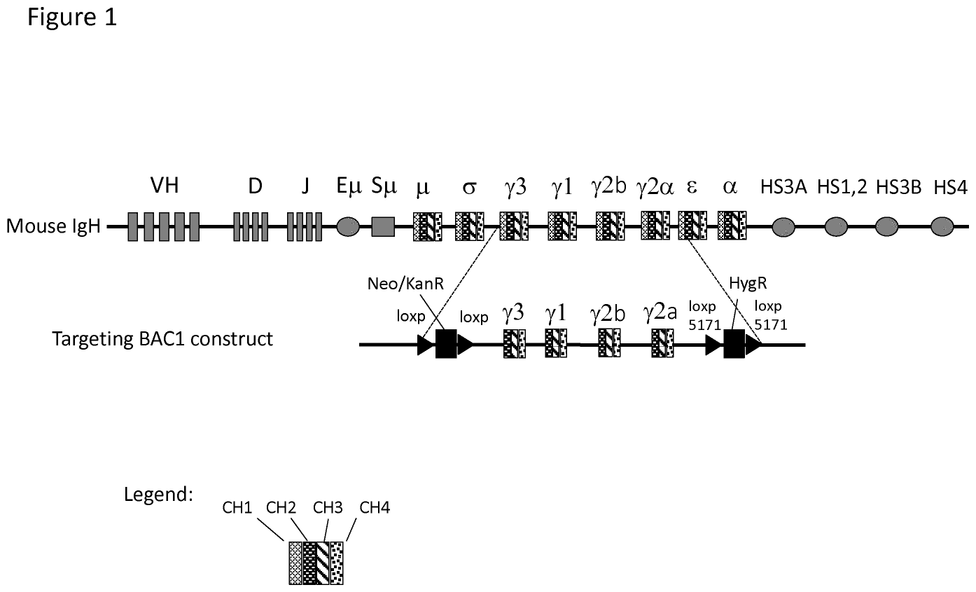

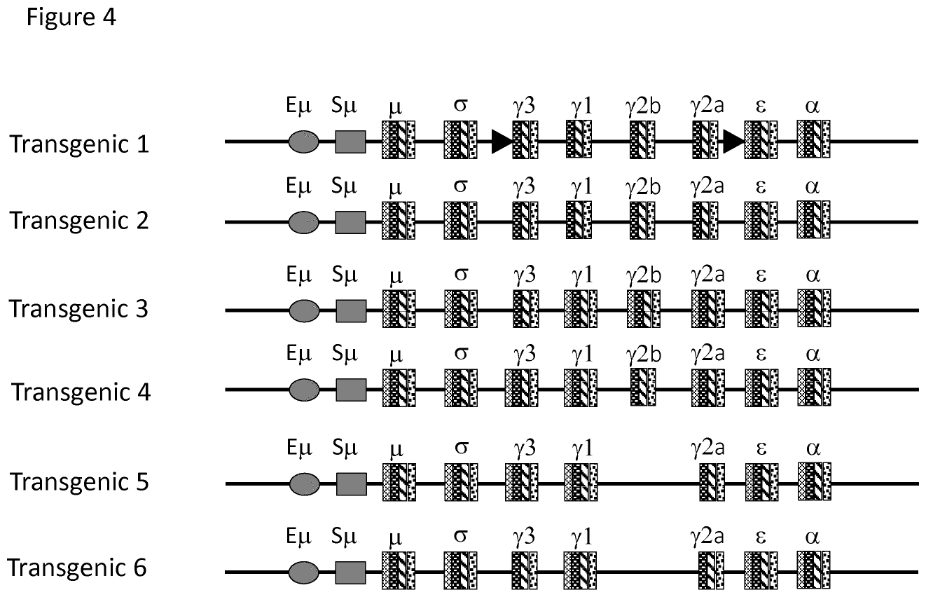

Genetically modified embryonic stem cells were obtained by targeted integration of BAC1 comprising deletion of the CH1 exon of each of γ3, γ1, γ2b and/or γ2a constant regions to the mouse IgH locus using Cre/Loxp recombination (FIG. 1). BAC1 and two constructs expressing Cas9 or single guide RNA (sgRNA) sequences targeting the inner mouse homologous regions were co-electroporated into the ES cells. Transfected ES cells were submitted to neomycin (G418) and Hygromycin selection. A total of 228 clones were isolated and screened by 5′ and 3′ long range PCR. The PCR positive clones were further analyzed by Southern blot analysis using both internal and external probes. A clone (#183) was confirmed with correct insertion (Transgenic 1 on FIG. 4).

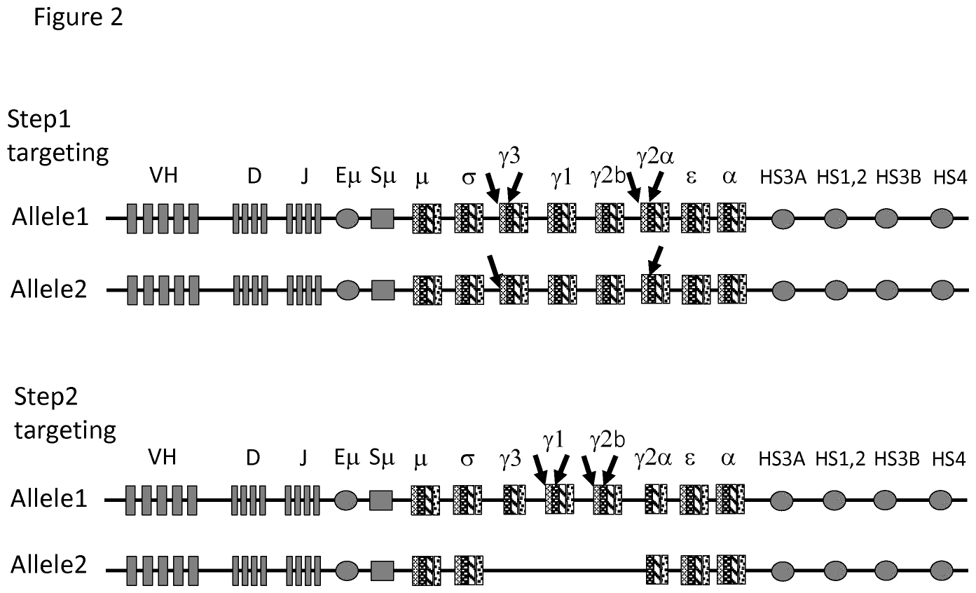

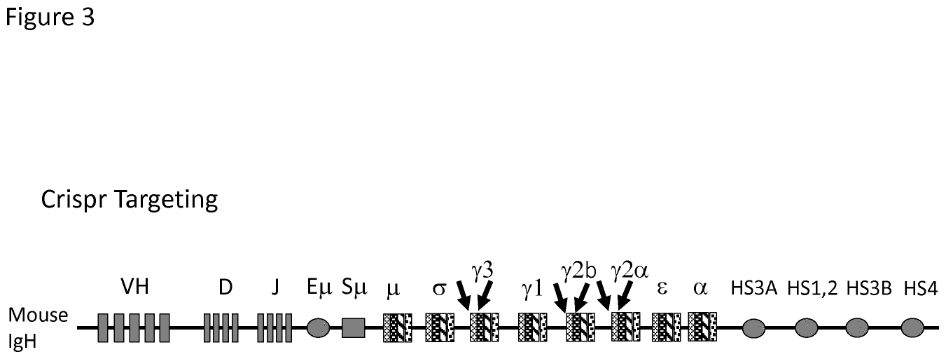

In parallel, CRISPR-targeted deletions of specific CH1 exons or of entire constant region genes were carried out in embryonic stem cells or in fertilized embryos (FIG. 2 and FIG. 3). Briefly, constructs expressing Cas9 and sgRNA sequences targeting the flanking regions of the CH1 exons of each of γ3, γ1, γ2b and/or γ2a constant regions were co-electroporated into the ES cell or injected into the pronuclear regions of fertilized mouse embryos. ES cell clones and injected animals were screened by PCR genotyping. One ES clone (13A10) with successful integration (Transgenic 2 on FIG. 4) was identified using the ES cell targeting approach. Four transgenic lines carrying variable mutations in the constant regions were identified (Transgenic 3, Transgenic 4, Transgenic 5, and Transgenic 6 in FIG. 4) using the embryo-targeting approach.

### Example 3—Generation of Transgenic Mice with Modified Constant Regions

Transgenic mice were obtained by implantation of blastocysts microinjected with ES clone 13A10 into a pseudopregnant mouse. Both ES clone 13A10 and blastocysts are under C57/B6 genetic background. Generation of chimeric FOs was confirmed by PCR genotyping. Chimeric mice were backcrossed with wild type C57/B6 animals to generate F1 heterozygous animals confirmed by PCR genotyping. Homozygous F2 animals were generated by F1 heterozygous crossing.

Alternatively, transgenic mice are obtained by implantation of blastocysts microinjected with ES clone #183 into a pseudopregnant mouse. High-degree chimeras were generated and bred with wild type C57/B6 animals to generate F1 heterozygous animals. PCR genotyping on F1 tail biopsy samples confirmed germline transmission of the desired mutations carried by BAC1 construct.

Using the approaches described in Example 2 and Example 3, heterozygous ES cells, heterozygous animals or homozygous animals having “Transgenic 1”, “Transgenic 2”, “Transgenic 3”, “Transgenic 4”, “Transgenic 5” or “Transgenic 6” genomic organization are obtained (FIG. 4). More particularly, homozygous transgenic animals having “Transgenic 2”, “Transgenic 3”, “Transgenic 4”, “Transgenic 5” or “Transgenic 6” genomic organization have been obtained and are referred to herein as “Transgenic 2 animal(s)”, “Transgenic 3 animal(s)”, “Transgenic 4 animal(s)”, “Transgenic 5 animal(s)” or “Transgenic 6 animal(s)”.

### Example 4—Expression of Single Domain Antibodies

Expression of heavy chains lacking CH1 domain or single domain antibodies from homozygous transgenic animals was verified by Western blot under reducing or non-reducing conditions using the experimental conditions exemplified below.

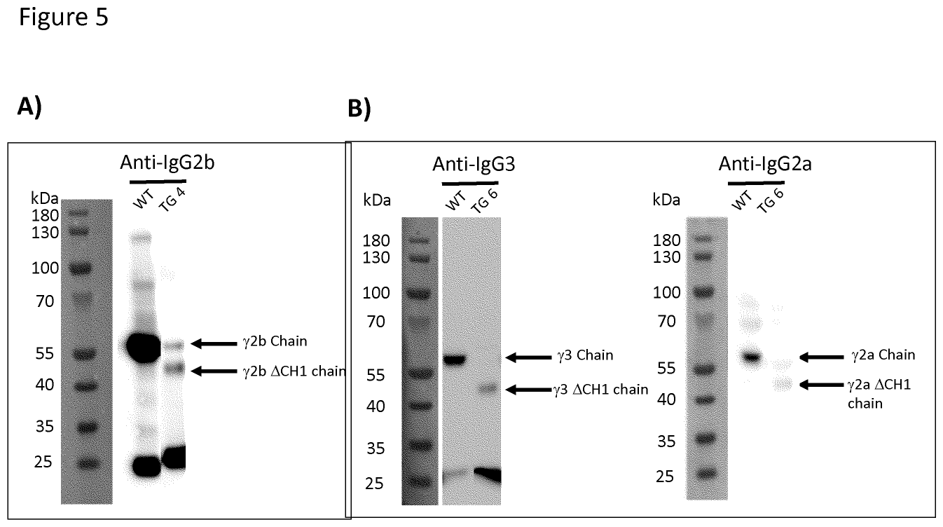

Briefly, serum samples were diluted at a ratio of 1/50 in water and 5 μL of diluted serum samples were loaded on gel (Bis-Tris 4-12%) under reducing conditions. Secondary antibodies of HRP-conjugated goat pAbs anti-mouse IgG2a (Abcam ab97245), HRP-conjugated goat pAbs anti-mouse IgG2b (Abcam ab97250), and HRP-conjugated goat pAbs anti-mouse IgG3 (Abcam ab97260) were used at 1/20,000 dilution for detection. The Transgenic 4 animal carrying CH1 deletion in γ2b showed expression of truncated IgG2b heavy chain (FIG. 5A). Transgenic 6 animal carrying CH1 deletions in both γ3 and γ2a showed expression of truncated IgG3 and IgG2a heavy chains (FIG. 5B).

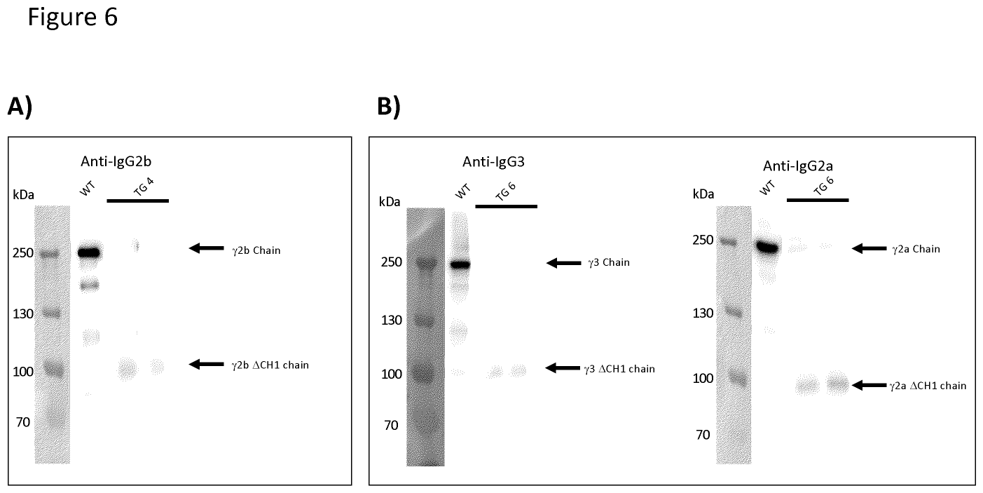

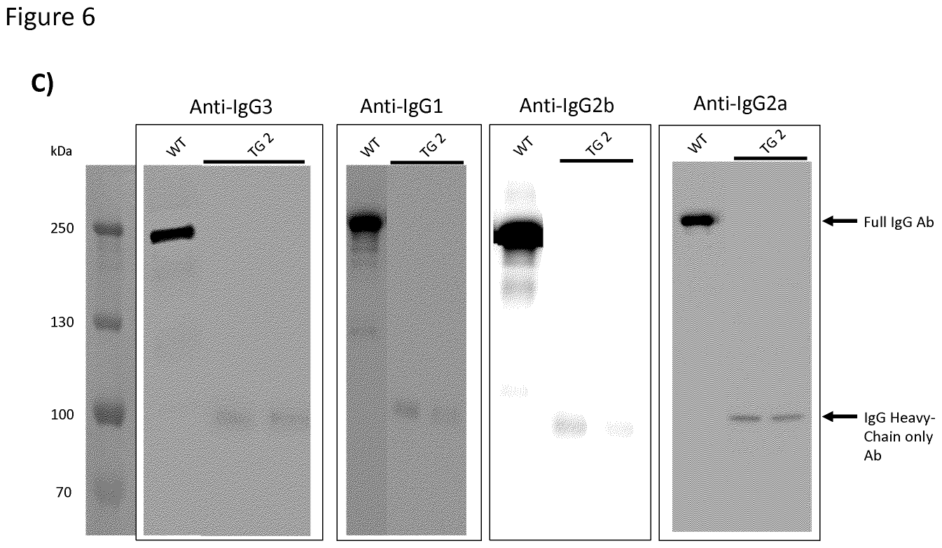

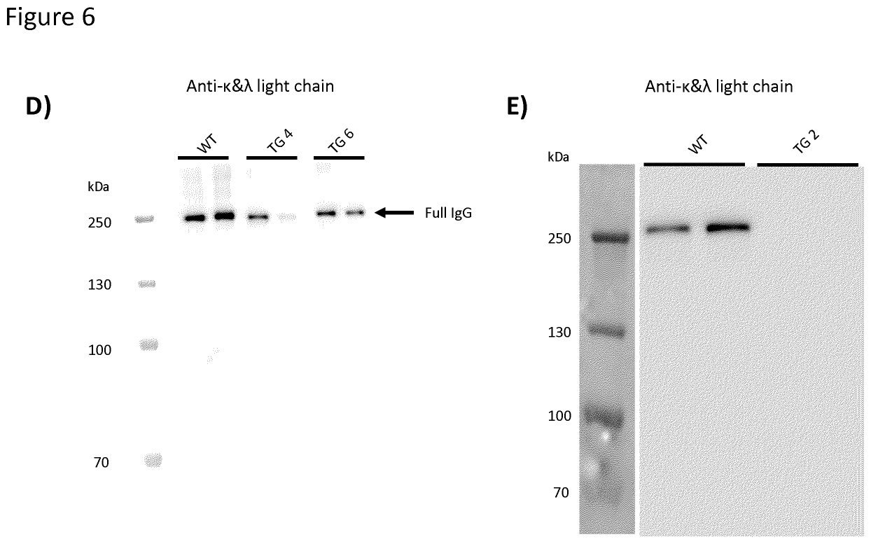

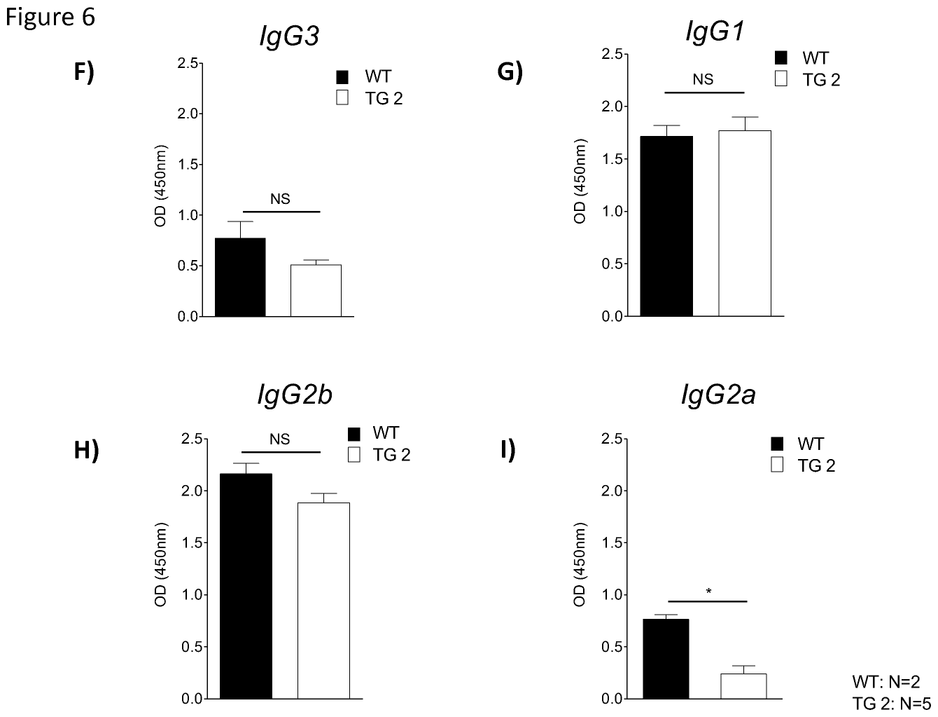

Alternatively, under non-reducing condition, serum samples were diluted at a ratio of 1/50 in water and 12 μL of diluted serum samples were loaded on gel (Tris glycine 8%). Secondary antibodies of RP-conjugated goat pAbs anti-mouse IgG2a (Abcam ab97245), RP-conjugated goat pAbs anti-mouse IgG2b (Abcam ab97250), and HRP-conjugated goat pAbs anti-mouse IgG3 (Abcam ab97260) were used at 1/10,000 dilution for detection or using RP-conjugated goat pAbs anti-mouse IgG light chain (Millipore, AP200P) at 1/10,000 dilution. The Transgenic 4 animal carrying CH1 deletion in γ2b showed expression of single domain antibodies IgG2b subclass (FIG. 6A). The Transgenic 6 animal carrying CH1 deletions in γ3 and γ2a showed expression of single domain antibodies from IgG3 and IgG2a subclasses (FIG. 6B). The Transgenic 2 animal carrying CH1 deletions in γ3, γ1, γ2b and γ2a showed expression of single domain antibodies from IgG3, IgG1, IgG2b and IgG2a subclasses (FIG. 6C).

In summary, our Western blot analysis on serum samples obtained from pre-immunized Transgenic 2 animal, Transgenic 4 animal or Transgenic 6 animal showed expression of heavy chains lacking CH1 domain (FIG. 5, reduced condition for Transgenic 4 animal and Transgenic 6 animal) and heavy chain only antibodies lacking CH1 domain (FIGS. 6A-6C, non-reduced condition for Transgenic 4 animal (FIG. 6A), Transgenic 6 animal (FIG. 6B) and for Transgenic 2 animal (FIG. 6C)).

Finally, Western blot analysis on serum samples obtained from pre-immunized Transgenic 2 animal, Transgenic 4 animal or Transgenic 6 animal did not show expression of single domain antibodies when detecting antibodies against κ and λ light chain were used, indicating the single domain antibodies expressed in Transgenic 2 animal, Transgenic 4 animal or Transgenic 6 animal do not associate with κ or λ light chain (FIG. 6D; non-reduced condition for Transgenic 4 animal and Transgenic 6 animal and FIG. 6E; non-reduced condition for Transgenic 2 animal).

Quantification of antibody subclass was performed with an ELISA detection kit. Serum samples were diluted at a concentration of 250 ng/μL in TBS and 50 μL of diluted antibodies were mixed with the detection antibodies which are specific to each of the IgG subclasses (Rapid ELISA Mouse mAb Isotyping Kit, cat. 37503, Thermofisher). Expression of each antibody subclass in the Transgenic 2 animals was compared to the wild type control serum sample. As illustrated in FIGS. 6F-6I, expression of IgG3, IgG1 and IgG2b subclass antibodies was comparable between Transgenic 2 animals and wild type control; expression of IgG2a subclass antibodies was decreased in Transgenic 2 animals compared to wild type control animals.

### Example 5—Expression of Single Domain Antibodies in Transgenic Mice Carrying CH1 Deletions

Expression of antigen-specific single domain antibody was assessed by immunizing transgenic animals with a tumor specific antigen (Target 1) expressed in human cells, with a control antigen expressed on immune cells (CD3; Target 2), or with a viral antigen (SARS-Cov-2 spike; Target 3).

Briefly, a group of 5 homozygous Transgenic 6 animals (female 8-12 weeks old) was used for an immunization experiment with a human target (Target 1). Before each immunization, 80-100 μL of blood samples were taken by tail bleeding to assess the titres of immunization response. For each immunization, 100 μL of emulsified human recombinant protein at concentration of 0.5 μg/μL was intraperitoneally injected. A total of 4 immunizations were performed during the course of 5 weeks. 3 days after the last injection, animals were sacrificed. Bone marrow and spleen tissues were collected. To make the phagemid library, total RNA was extracted from bone marrow and spleen tissues collected from immunized animals and was made into cDNA samples. Mouse VH-specific primers were used to amplify the sdAb variable sequences using cDNA templates. Then the total sdAb amplicon library was subcloned into pMECS-GG vector, and electroporated into E. coli TG1 cells (Agilent 200123) to make the phagemid library.

A series of panning were performed with the phagemid immune library. In brief, the first three rounds of panning were done using human recombinant Target 1 protein with a control protein, followed by a fourth round of panning done using a Target 1-positive cell line and a control of a Target 1-negative cell line. Both random cloning picking and NGS analysis were conducted after completion of panning.

Serum samples were collected and dilutions of 1/100 and 1/15000 were used to evaluate the antibody titre of anti-Target 1 single domain antibodies by ELISA. Secondary goat anti-mouse IgG antibody (Jackson immunoresearch, 111-035-062) was used to detect serum antibodies. Western blot experiments were carried out to detect IgG2a or IgG3 antibody subclasses. The blot was blocked with 5% milk/PBS-Tween 0.1% and then was probed with a polyclonal goat anti-mouse IgG2a or IgG3 antibody (ab97245, ab97260, Abcam).

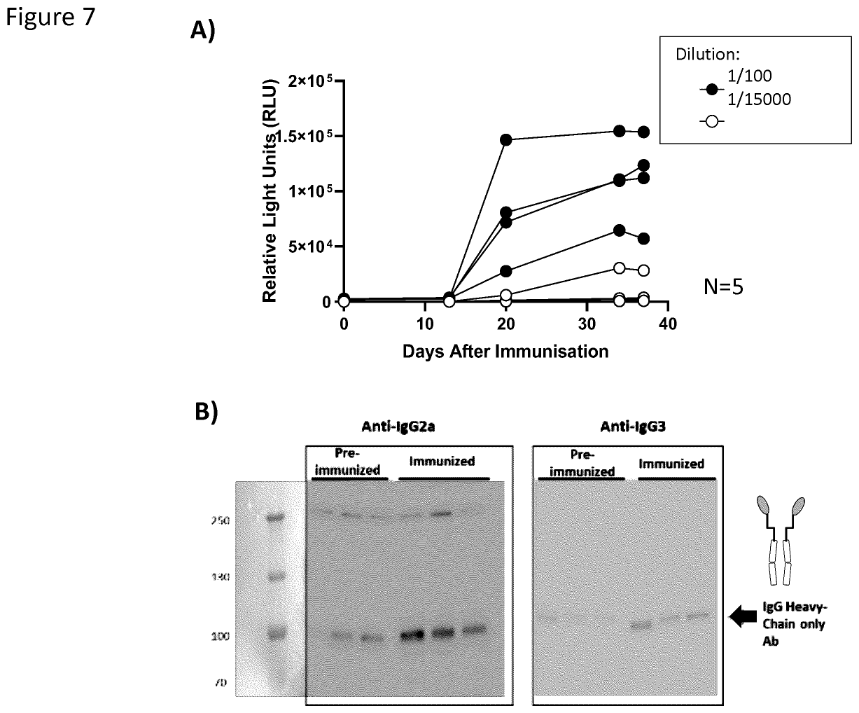

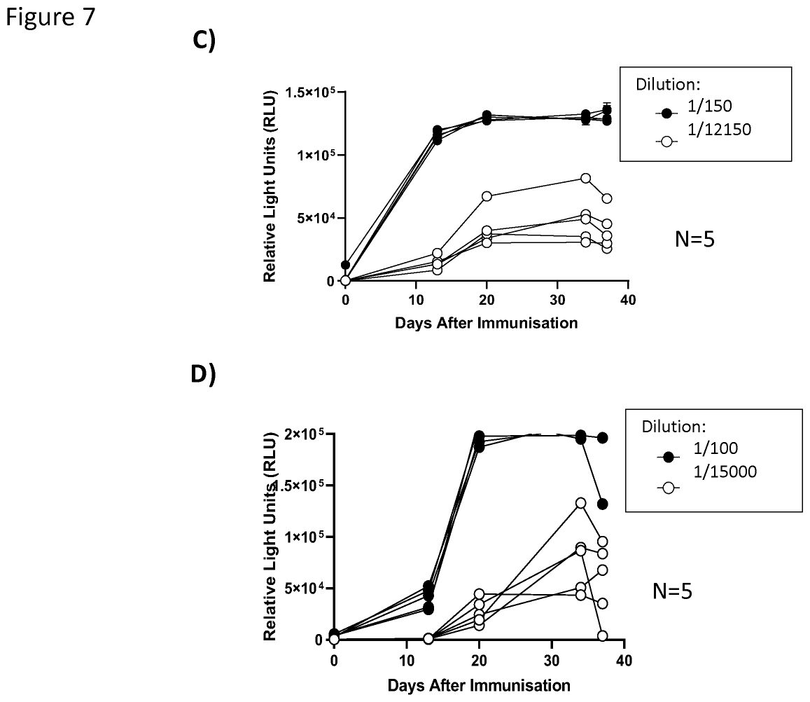

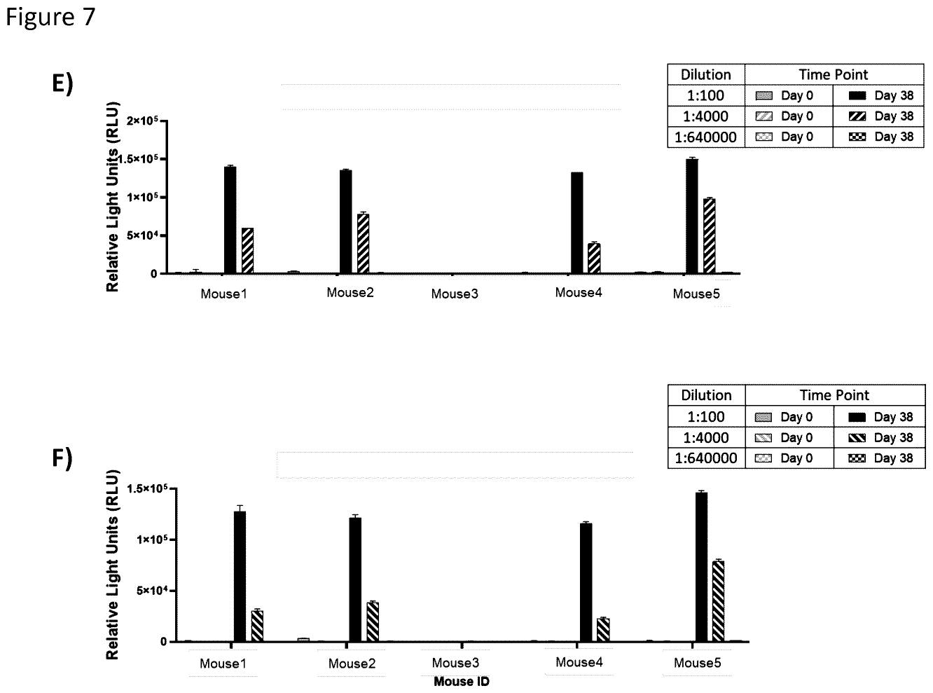

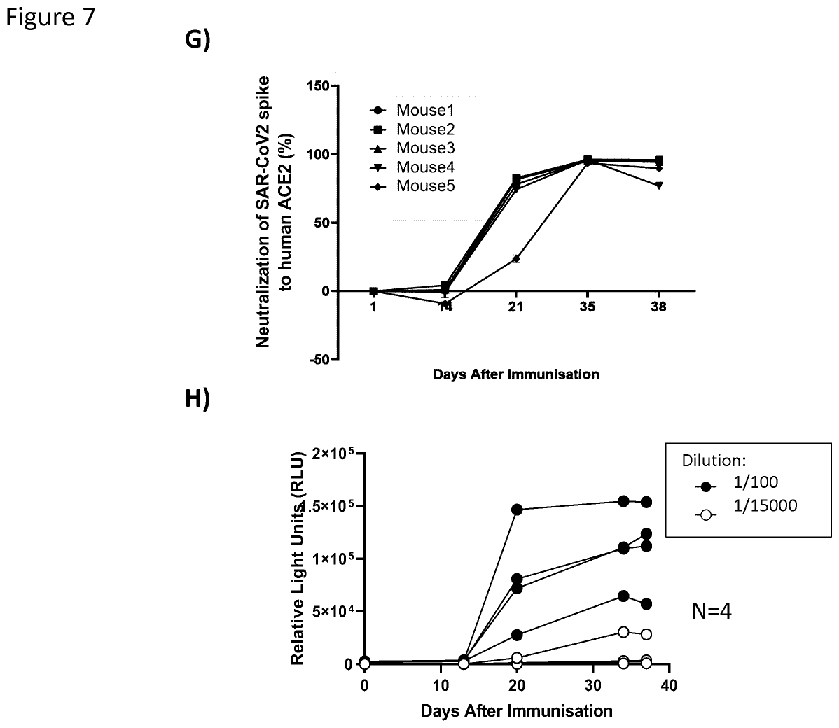

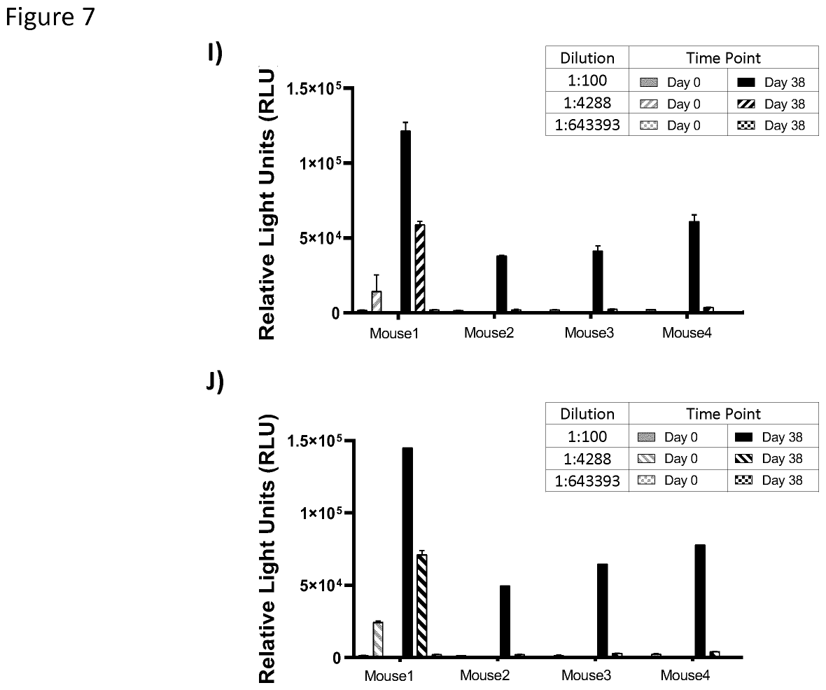

As illustrated in FIG. 7A, ELISA detection showed increased antibodies to Target 1 from serum samples collected from immunized transgenic animals and the titre was maintained at steady level after second immunization boost. Antibody titres remained detectable by ELISA after 15000-fold dilution of serum. Serum samples collected before and after the immunization of Target 1 were used to evaluate sdAb expression. As illustrated in FIG. 7B, increased sdAb expression was observed by Western blot in post-immunization samples for both IgG2a and IgG3 subclasses.

A group of 5 homozygous Transgenic 6 animals was used for an immunization experiment with CD3 as an antigen (Target 2). Briefly, each transgenic mouse was intraperitoneally injected with 100 μL of emulsified human recombinant proteins of CD3 ε and δ subunits at concentration of 0.5 g/μL. A total of 4 immunization injections were performed during the course of 5 weeks. 3 days after the last injection, animals were sacrificed. Serum sample and spleen tissues were collected and RNA extracted to construct a library of variable heavy chains (VHs). Two rounds of panning were performed against the recombinant human CD3 ε and δ subunits using phage display. DNA samples were collected and analyzed by NGS (Miseq, v600 cycle, 25 million reads). In parallel, 96 phage clones were picked from the second round of panning and tested for binding to recombinant human CD3 protein by ELISA and binding to human PBMCs by flow cytometry. The nucleic acid of positive binders was obtained and used to generate antibodies or antibody-like molecules. Serum samples were collected and dilutions of 1/150 and 1/12150 were used to evaluate the antibody titre of anti-CD3 single domain antibodies by ELISA.

As illustrated in FIG. 7C, anti-CD3 antibodies are produced from Transgenic 6 animals. The antibody titre was maintained at steady level after second immunization injection. Antibody titres remained detectable by ELISA after >12000-fold dilution of serum.

A group of 5 homozygous Transgenic 6 animals was used for an immunization experiment with wild type SARS-CoV-2 spike protein (UniProtKB accession: P0DTC2) as an antigen (Target 3). Briefly, each transgenic mouse was intraperitoneally injected with 100 μL of emulsified spike proteins at concentration of 0.5 μg/μL. A total of 4 immunization injections was performed during the course of 5 weeks. 3 days after the last injection, animals were sacrificed. Serum sample and spleen tissues were collected and RNA extracted to construct a library of variable heavy chains (VHs). Serum samples were collected and dilutions of 1/100 and 1/15000 were used to evaluate the antibody titres of anti-Target 3 single domain antibodies by ELISA.

As illustrated in FIG. 7D, anti-spike antibodies were produced from Transgenic 6 animals. The antibody titre was maintained at steady level after third immunization injection. Antibody titres remained detectable by ELISA at 1/15000 dilution of serum.

To assess whether sdAbs generated from Transgenic 6 animals immunized with wild type SARS-CoV-2 spike protein would cross-react with other spike protein variants, day 38 serum samples from all five animals were tested by ELISA for their binding to spike glycoprotein variant B.1.351 (Beta) (FIG. 7E), and spike glycoprotein variant B.1.1.7 (Alpha) (FIG. 7F). In both tests, serum sample from mouse 1, 2, 4, and 5 showed binding to both variant proteins at lower and higher dilutions (1:100 and 1:4000).

To assess neutralization of SARS-CoV-2 spike glycoprotein, day 38 serum of five Transgenic 6 animals, immunized with wild type SARS-CoV-2 spike glycoprotein, was diluted in blocking buffer to 1/350 and tested by a neutralization assay (FIG. 7G). In brief, serum samples were incubated with HRP-conjugated-Spike-glycoprotein binding domain (RBD) at 1:1 volume ratio, then the mixture was added to a hACE2-pre-coated plate and incubated for 15 minutes at room temperature. After incubation, the wells were washed four times with washing buffer. The wells were incubated for 15 minutes at room temperature with TMB solution then stop solution was added to quench the reaction. The plate was read immediately on a SpectraMax™ i3x Multi-Mode Microplate Reader (Molecular Devices). The percent inhibition was calculated using data from day 1 as the baseline to serum collected at different points of immunization. Serum from mice 1, 2, and 3 collected at day 38 competed with RBD and hACE2 and showed >94% inhibition. Serum from mice 4 and 5 collected at day 38 competed with RBD and hACE2 and showed >77% inhibition.

A group of 4 homozygous Transgenic 2 animals were used for an immunization experiment with wild type SARS-CoV-2 spike protein as an antigen (Target 3). Briefly, each transgenic mouse was intraperitoneally injected with 100 μL of emulsified spike proteins at concentration of 0.5 g/μL. A total of 4 immunization injections was performed during the course of 5 weeks. 3 days after the last injection, animals were sacrificed. Serum samples and spleen tissues were collected and RNA extracted to construct a library of variable heavy chains (VHs). Serum samples were collected and dilutions of 1/100 and 1/15000 were used to evaluate the antibody titres of anti-Target 3 single domain antibodies by ELISA.

As illustrated in FIG. 7H, anti-spike antibodies were produced from Transgenic 2 animals. The antibody titre was maintained at steady level after third immunization injection. Antibody titres remained detectable by ELISA at 1/15000 dilution of serum for 2 of 4 animals.

To assess whether sdAbs generated from Transgenic 2 animals immunized with wild type SARS-CoV-2 spike protein would cross-react with other SARS-CoV-2 spike protein variants, day 38 serum samples from all four animals were tested by ELISA for their binding to spike glycoprotein variant B.1.351 (Beta) (FIG. 7I), and spike glycoprotein variant B.1.1.7 (Alpha) (FIG. 7J). In both tests, serum sample from mouse 1 showed binding to both variant proteins at lower and higher dilutions (1:100 and 1:4288), serum samples from mouse 2, 3, and 4 showed binding to both variant proteins only at lower dilution (1:100).

To assess neutralization of SARS-CoV-2 spike glycoprotein, day 38 serum of four Transgenic 2 animals, immunized with wild type SARS-CoV-2 spike glycoprotein, is diluted in blocking buffer to 1/350 and tested by a neutralization assay. Sera are incubated with HRP-conjugated-Spike-glycoprotein binding domain (RBD) then added to a hACE2-pre-coated plate. The percent inhibition is calculated using data from day 1.

In conclusion, immunization of two HCAb transgenic animals (Transgenic 2 and Transgenic 6) disclosed herein with Target 1, CD3 (Target 2), or SARS-CoV-2 wild type spike (Target 3) resulted in successful expression of target-specific single domain antibodies.

### Example 6—Characterization of Antibody Library Obtained by Immunization of Transgenic Mice Carrying CH1 Deletions

DNA samples from Transgenic 6 animals (4 immune libraries from 4 transgenic mice) and alpaca (2 immune libraries from two alpacas) immunized with Target 1 were carried out with amplicon based NGS analysis (Miseq V3, 600 cycles). A total of 2 million of paired reads were obtained from the two alpaca library samples, and a total of 4.7 million of paired reads were obtained from the four transgenic 6 library samples by NGS sequencing.

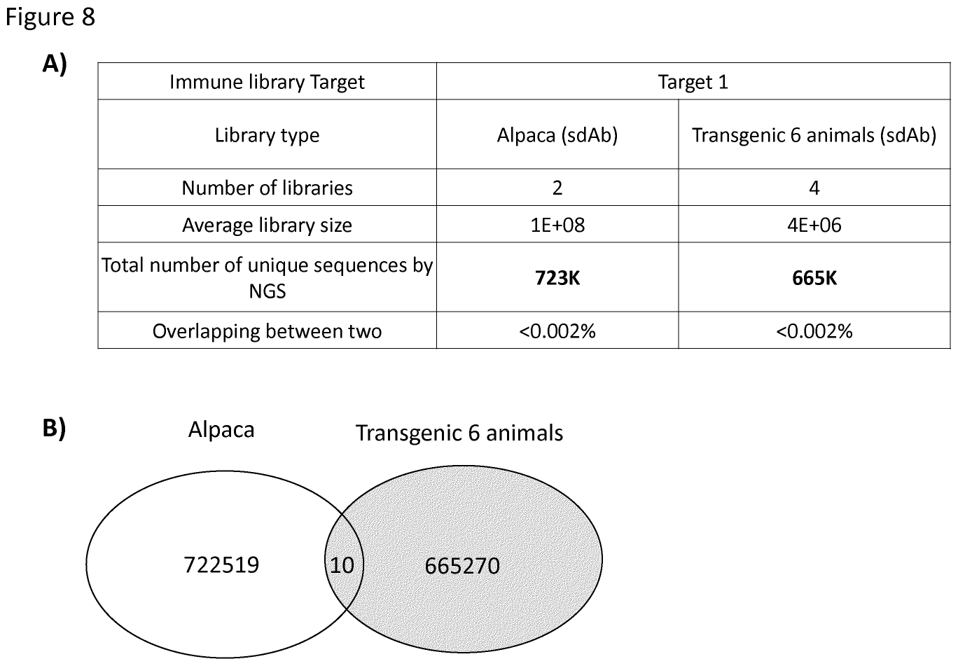

As illustrated in FIG. 8A, the total number of unique sequences was comparable between the transgenic mice and alpaca immune libraries (723,000 in alpaca versus 665,000 in transgenic mouse). The size of the immune library obtained from the Transgenic 6 animals was smaller compared to the alpaca immune libraries (4E+6 in Transgenic 6 animal libraries versus 1E+8 in alpaca libraries).

As illustrated in FIG. 8B, few sequences overlap between Transgenic 6 immune libraries and alpaca immune libraries. Only 10 sequences with 100% sequence identity were found in both libraries.

These results demonstrate that antibodies derived from transgenic animals show little or no sequence overlap to those of alpaca, suggesting a unique antibody repertoire to Target 1, despite being immunized with the same antigen. Though a smaller size of library was generated from Transgenic 6 animals, the number of unique sequences (non-identical sdAb sequences by NGS) was comparable between two species. This suggests a higher complexity of the transgenic animal's immune response to Target 1.

All unique sequences from the four Transgenic 6 immune libraries were assessed for the four camelid VHH hallmark mutations at positions 37, 44, 45, and 47. The percentage was calculated as the total number of sequences with all four camelid VHH mutations/total number of unique sequences. The camelid VHH canonical framework mutations (Table 2) could be detected in all four Transgenic 6 immune libraries. sdAbs containing the VHH hallmark mutations at all four positions ranged from 0.03% to 0.06%. A higher percentage of sdAbs contained at least one of these mutations.

TABLE 2

| Position | 37 | 44 | 45 | 47 |
|---|---|---|---|---|
| Camelid VHH | F/Y | E/Q | R/C | F/G/L/W |
| consensus | | | | |
| Mouse VH | V | G/A/R/S/K | L/P | W |
| consensus | | | | |

Camelid VHH framework mutations help decrease the hydrophobicity, thus, increase the stability of the sdAb structure. With NGS deep sequencing, it was demonstrated that Transgenic 6 animals produced sdAbs with canonical camelid framework mutations possibly due to mutation events during antibody maturation.

### Example 7—Characterization of Selected Single Domain Antibody Species Obtained by Immunization of Transgenic Mice Carrying CH1 Deletions

The variable region of ten randomly picked sdAbs was fused with human IgG1 Fc. The resulting homodimeric binding agents (sdAb-Fc1 to sdAb-Fc10) were produced and isolated from cells and were evaluated by ELISA for binding specificity and affinity for Target 1. In addition, recombinant proteins (recombinant Target 1) from different species were used to assess the antibody cross-reactivity. All antibodies were tested with a single concentration of 357 nM, followed by detection with secondary goat anti-human IgG-Fc antibody (Jackson immune research, cat #109-035-098) at 1/5000 dilution.

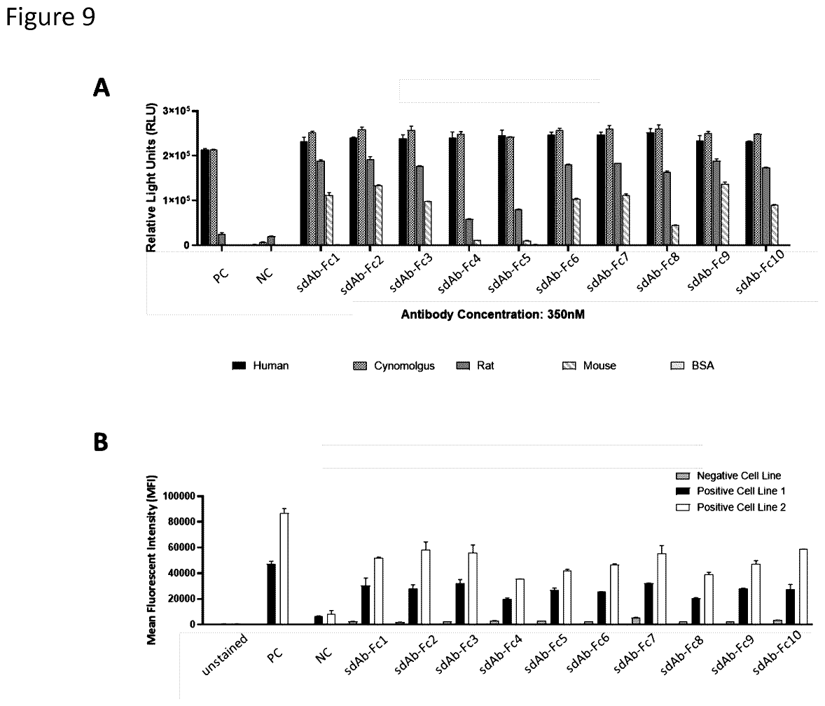

As illustrated in FIG. 9A, all 10 sdAb-Fcs bind to human Target 1 recombinant protein. 8 out of 10 sdAb-Fcs showed cross-reactivity towards Target 1 from all four species tested.

Two Target 1-positive cell lines and one negative cell line were used to assess the binding of all 10 sdAb-Fcs derived from the Transgenic 6 immune library. In each well, 100,000 cells were used to incubate with sdAb-Fc at concentration of 714 nM. Then anti-human IgG Fc antibody (Biolegend, cat #409306) at 1/500 dilution was used as secondary antibody, followed by incubation with human 7AAD (Biolegend, cat #422302) before FACS.

As illustrated in FIG. 9B, all 10 sdAb-Fcs showed specific binding to the Target 1-positive cell lines but not to the negative control.

These data indicate that the variable region sequences derived from Transgenic 6 immune libraries can be used to generate binding agents to Target 1.

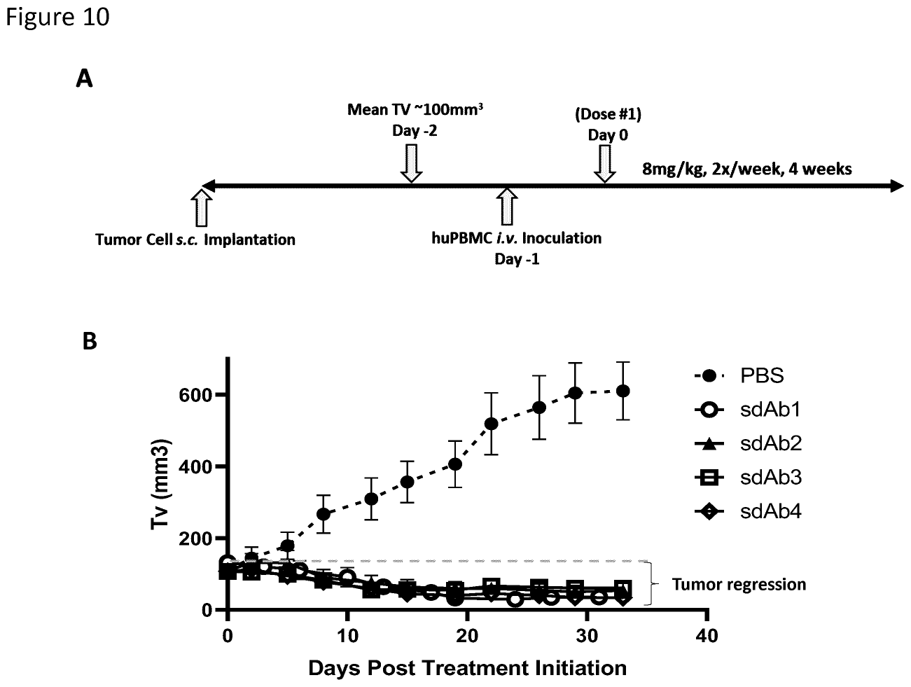

We further selected 4 sdAb-Fcs from the 10 positive binders to evaluate their anti-cancer efficacy using an established triple-negative breast tumor model, MDA-MB-453, in NCG mice. Each treatment group contained 6 NCG mice (female, 5-6 weeks of age, Charles Rivers Laboratories). 5 million of MDA-MB-453 breast tumor cells were subcutaneously injected into mice. When tumor volume reached at least 100 mm³ in size, 10 million of human PBMCs were inoculated by intravenous injection (day −1). On next day (day 0), the mice were randomized into to two groups and for each group antibodies were injected intraperitoneally at a dosage of 8 mg/kg, or PBS two times per week for a total of 4 weeks (treatment scheme illustrated in FIG. 10A). As illustrated in FIG. 10B, tumor regression was observed in animals treated with all 4 sdAb-Fc antibodies.

These data indicate that the variable region sequences derived from Transgenic 6 animal immune libraries can be used to generate functionally active binding agents to Target 1 as all 4 sdAb-Fcs caused tumor regression.

### Example 8—Selection of Sequences from Alpaca, Bactrian, Llama and Dromedary IgH Locus (De Novo Sequencing)

Genomic DNA (gDNA) was extracted from testis tissues of alpaca, Bactrian, llama and dromedary. Purified gDNA samples were digested with HindIII enzyme. Digested fragments of more than 100 Kb were used for constructing distinct BAC libraries for all four species. Primers were designed based on the consensus sequences of the V segments, D segments, J segments, and IgM constant region of each species and used to screen and isolate positive clones from the BAC libraries. For each library, 6 positive clones to the V segments but negative to D and J segments, 2 positive clones to V, D, J segments and IgM constant region, and 2 positive clones to the IgM constant region but negative to J segments were selected and submitted to Single Molecular Real-time (SMRT) sequencing (PacBio). Using this approach, 445 Kb of partial alpaca IgH genomic sequences was identified including a 223 Kb fragment identified previously (GenBank ID AM773729.1, https://www.ncbi.nlm.nih.gov/nuccore/AM773729); 455 kb of llama partial IgH genomic sequences, 445 kb of dromedary partial IgH genomic sequences, and over 773 Kb of Bactrian partial IgH genomic sequences were identified by SMRT sequencing, respectively. To the Applicant's knowledge, these large Bactrian and llama genomic IgH locus sequences have been uncovered by the Applicant for the first time.

Each camelid V segment includes approximately 5 kb upstream and 5 kb downstream of the V segment and therefore includes regulatory sequences, intronic sequences, leader sequences and recombination signal sequences associated with the V segment. Therefore, each camelid V gene segments comprises the originals regulatory sequences that is associated with such V segment in the camelid genomic DNA.

V segments encoding VHs and VHHs were cloned into bacterial artificial chromosomes for generation of ES cells and transgenic animals.

### Example 9—Generation of ES Clones and Transgenic Mice Having Modified Variable Regions

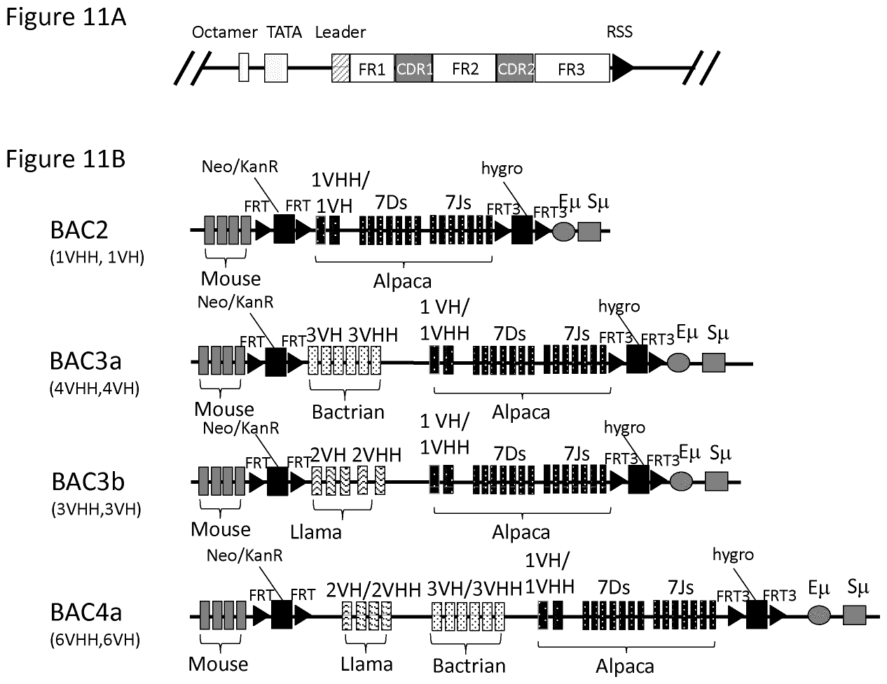

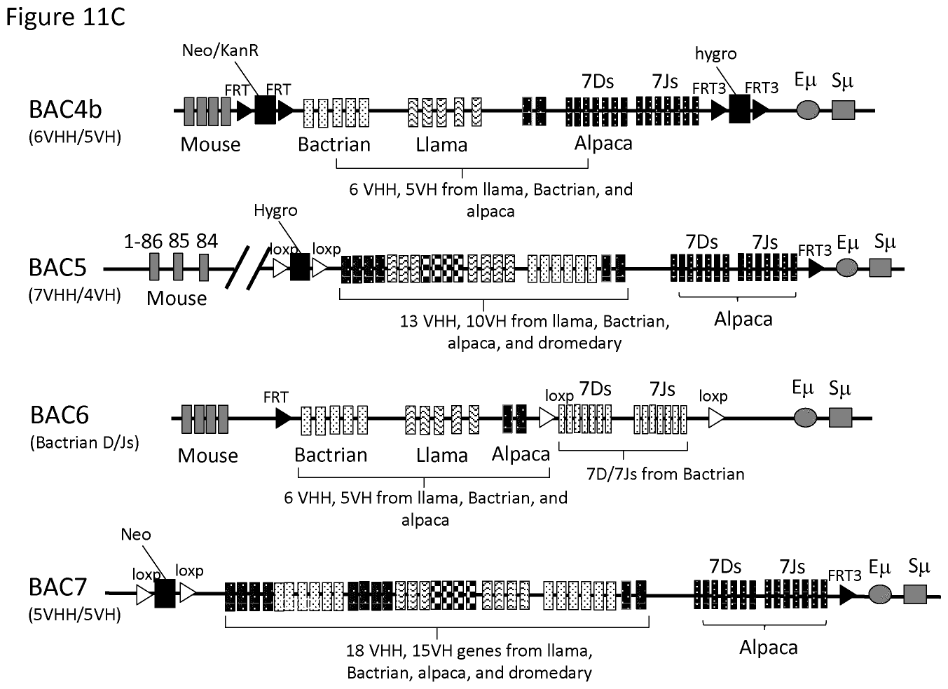

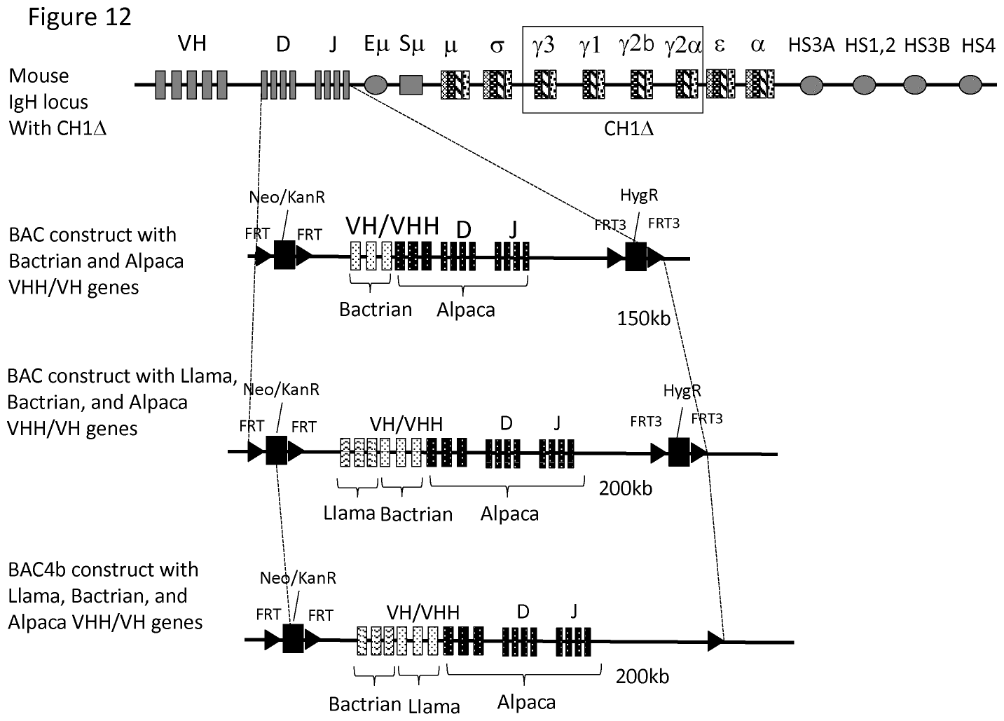

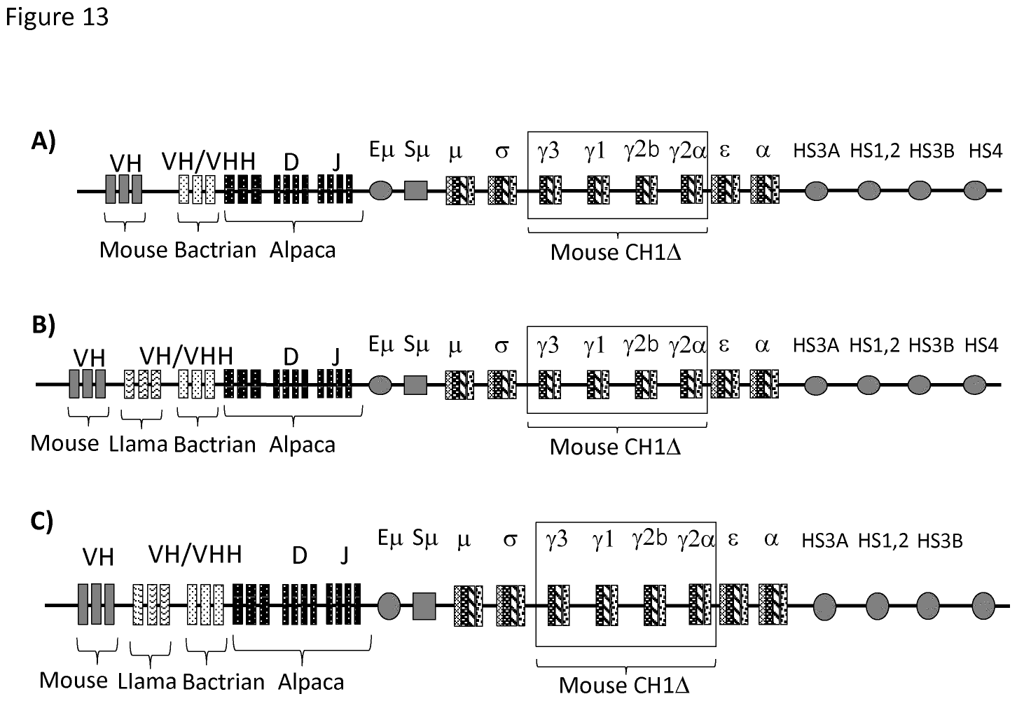

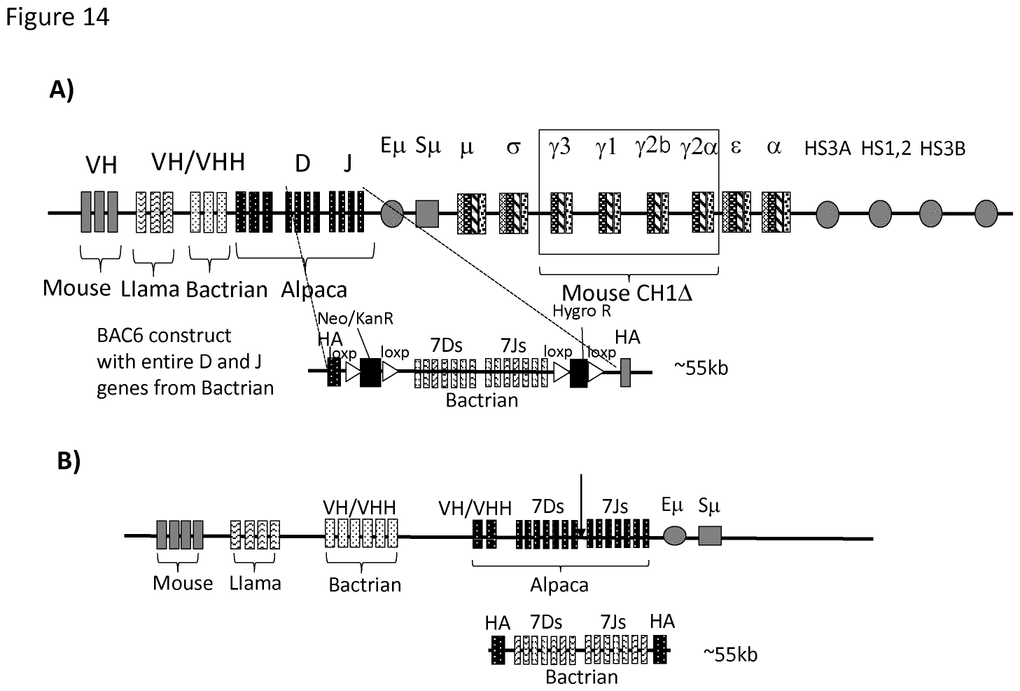

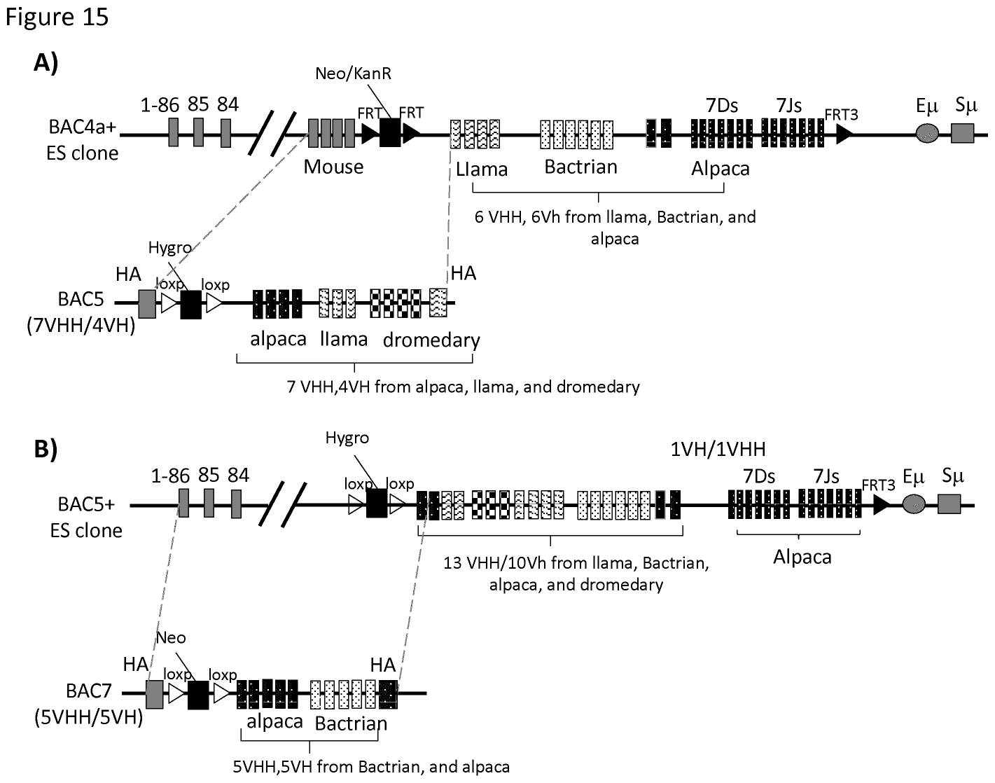

ES cell clones and transgenic mice expressing camelid VH, VHH, D and J sequences are generated by single or successive targeted integration of bacterial artificial chromosomes (e.g., BAC2, BAC3a, BAC3b, BAC4a, BAC4b, BAC5, BAC6, BAC7) in ES cells carrying desired CH1 deletion(s) as exemplified in FIGS. 12, 14 and 15 resulting in ES cell clones and transgenic mice carrying the transgenes exemplified in FIG. 11B, FIG. 11C and FIG. 13

For example, murine ES cells carrying deletion of the CH1 exon of each of the γ3, γ1, γ2b and γ2a constant region gene (as illustrated by Transgenic 2 in FIG. 4) are transfected with BAC constructs comprising camelid V, D and/or J segments and ES clones carrying the proper recombination events (exemplified in FIG. 11B, 11C or 13) are used for making chimeric animals. Chimeric animals are obtained by implantation of blastocysts microinjected with selected ES clone into a pseudopregnant mouse. Chimeric mice are backcrossed to wild type C57/B6 animals to generate F1 heterozygous animals confirmed by PCR genotyping. Homozygous F2 animals are generated by F1 heterozygous crossing.

More particularly, the BAC2 construct was used to target and replace the endogenous mouse D and J gene segments with alpaca D and J gene segments at the mouse IgH locus. Targeted integration of the BAC2 construct was confirmed by PCR genotyping. ES cell clones, comprising the BAC2 transgene illustrated in FIG. 11B were selected. The construct is used as an intermediate for constructing additional BACs including BaC3a.

The BAC3a construct was used to target and replace the endogenous mouse D and J genes and to add camelid VHs and VHHs at the IgH locus. Targeted integration of the BAC3a construct was confirmed by PCR genotyping. ES cell clones, comprising the BAC3a transgene illustrated in FIG. 11B were selected. The construct is used as an intermediate for constructing additional BACs including BaC4a.

The BAC3b construct was used to target and replace the endogenous mouse D and J genes and to add camelid VHs and VHHs at the IgH locus. Targeted integration of the BAC3b construct was confirmed by PCR genotyping. ES cell clones, comprising the BAC3b transgene illustrated in FIG. 11B were selected. The construct is used as an intermediate for constructing additional BACs including BaC4b.

The BAC4a construct and constructs expressing Cas9 or single guide RNA (sgRNA) sequences targeting the inner regions of the mouse homologous arms were co-electroporated into the Δ CH1 ES cell clone 13A10 (carrying Transgene 2). Transfected ES cells were subjected to neomycin (G418). BAC4a targets and replaces the endogenous mouse D and J genes and adds camelid VHs and VHHs at the IgH locus. A total of 228 clones were isolated and screened by 5′ and 3′ long range PCR. Targeted integration of the BAC4a construct was confirmed by PCR genotyping. ES cell clones, comprising the BAC4a transgene illustrated in FIG. 11B including clone 1F4, 3H5, and 1F10 were selected for blastocyst injection. 54/60 survived blastocysts were born. PCR genotyping was performed on tail biopsy samples to confirm presence of the transgene.

The BAC4b construct was electroporated into the Δ CH1 ES cell clone 13A10 (carrying Transgene 2) to target and replace the endogenous mouse D and J genes and to add camelid VHs and VHHs at the IgH locus. After PCR screening, several BAC4b-positive ES clones (>20), including clones 1D4, 5D10, and 5C4 were identified and confirmed for targeted integration of the BAC4b construct as illustrated in FIG. 11C. Clones 1D4, 5D10, and 5C4 were selected for blastocyst injection. 15/40 survived blastocysts were born. A male chimera derived from clone 5D10 was set up with wild type female and yielded a litter of 8 pups. 5 out 8 animals transmitted the BAC4b gene confirmed by PCR genotyping on the tail biopsy samples.

The BAC5 construct was electroporated into the BAC4a-positive ES cell clones 1F4, 3H5, and/or 1F10 for targeted replacement of mouse VH segments and integration of additional VHHs from alpaca, Llama and dromedary. After PCR screening, BAC5-positive ES clones were identified and confirmed for targeted integration of the BAC5 construct as illustrated in FIG. 11C. Clones were selected for further experiments.

The BAC6 construct was electroporated into the BAC4b-positive ES cell clone 5D10 for targeted replacement of alpaca D/J segments with Bactrian D/J segments. After PCR screening, several BAC6-positive ES clones were identified and confirmed for targeted integration of the BAC6 construct as illustrated in FIG. 11C. Clones 1B10, 1E6, and 1D5 were used for blastocyst injection. A total of 24 pups were born.

The BAC7 construct was electroporated into the BAC5 positive ES cell clone for targeted removal of all mouse VHs and insertion of additional alpaca and Bactrian VHHs. After PCR screening, positive BAC7 ES clones are identified and confirmed for targeted integration of the BAC7 construct as illustrated in FIG. 11C. Positive clones were selected for further experiments.

The BAC5 and BAC7 constructs containing 13 novel VHH genes from alpaca, llama, Bactrian and dromedary camels replace the entire mouse endogenous VH genes on IgH locus. In the BAC5 construct, VHH genes from dromedary, a fourth camelid species, were introduced to increase the diversity of camelid VHH gene repertoire. BAC5 and BAC7 were introduced in a stepwise way to target the mouse IgH locus.

The BAC5 construct was introduced first to remove the endogenous Neomycin resistance marker from the BAC4a positive ES cell clone. Bac5 contains a Hygromycin resistance gene cassette to enable selection of Bac5 positive ES cell clones. The BAC7 construct was then introduced into BAC5 positive ES clone(s) to targeted remove the endogenous Neomycin resistance marker on the BAC5 positive ES clone. A Neomycin resistance gene cassette on the Bac7 construct was used to select Bac7 positive ES cell clones. Complete PCR screening was carried out to confirm positive ES cell clones used for blastocyst injection.

Integration of the BAC2, BAC3a, BAC3b, BAC4a, BAC4b, BAC5, BAC6, and/or BAC7 construct in ES cell clones carrying CH1 deletion (e.g., Transgene 2) therefore results in ES cell clones comprising the transgenes illustrated in FIG. 11B, FIG. 11C and FIG. 13 (the Transgene 2 constant region is not illustrated for conciseness). ES cell clones positive for the desired transgene were expanded and stored for further experiments.

Transgenic mice are obtained as described in Example 3. The transgenic mice (chimeric, heterozygous or homozygous) were immunized with a desired antigen in order to produce antigen-specific single domain antibodies.

### Example 10—Expression of Single Domain Antibodies Carrying Camelid V, D and/or J Domains

Western blot experiments were performed on serum samples of pre-immunized animals carrying camelid V, D and J segments under reducing or non-reducing conditions. Briefly, under reducing conditions, serum samples were diluted at a ratio of 1/50 in water and 5 μL of diluted serum samples was loaded on gel (Bis-Tris 4-12%). Secondary antibodies such as HRP-conjugated goat pAbs anti-mouse IgG2a (Abcam ab97245), goat pAbs anti-mouse IgG2b HRP (Abcam ab97250), and RP-conjugated goat pAbs anti-mouse IgG3 (Abcam ab97260) were used at 1/20,000 dilution for detection. Under non-reducing conditions, serum samples were diluted at a ratio of 1/50 in water and 12 μL of diluted serum samples was loaded on gel (Tris glycine 8%). Secondary antibodies such as RP-conjugated goat pAbs anti-mouse IgG2a (Abcam ab97245), HRP-conjugated goat pAbs anti-mouse IgG2b (Abcam ab97250), and HRP-conjugated goat pAbs anti-mouse IgG3 (Abcam ab97260) were used at 1/10,000 dilution for detection.

Quantification of antibody subclass was performed by ELISA or flow cytometry. Serum samples were diluted at a concentration of 250 ng/μL in TBS and 50 μL of diluted antibodies was mixed with detection antibodies that are specific to each of the IgG subclasses (Rapid ELISA Mouse mAb Isotyping Kit, cat. 37503, Thermofisher). Expression of each subclass antibodies in the transgenic animals was normalized to the wild type control serum sample.

Western blot experiments were performed as described above to evaluate expression of camelid sdAb in BAC4b chimeric animals. 2 μl of each mice serum was used under non-reduced conditions and transferred to a nitrocellulose membrane using a semi-dry system. The blot was blocked with 5% milk/PBS-Tween 0.1% and then was probed with a rabbit monoclonal anti-Camelid antibody (A01861, Genscript) followed by an RP-conjugated goat anti-rabbit IgG (H+L) antibody (111-035-045, Jackson ImmunoResearch).

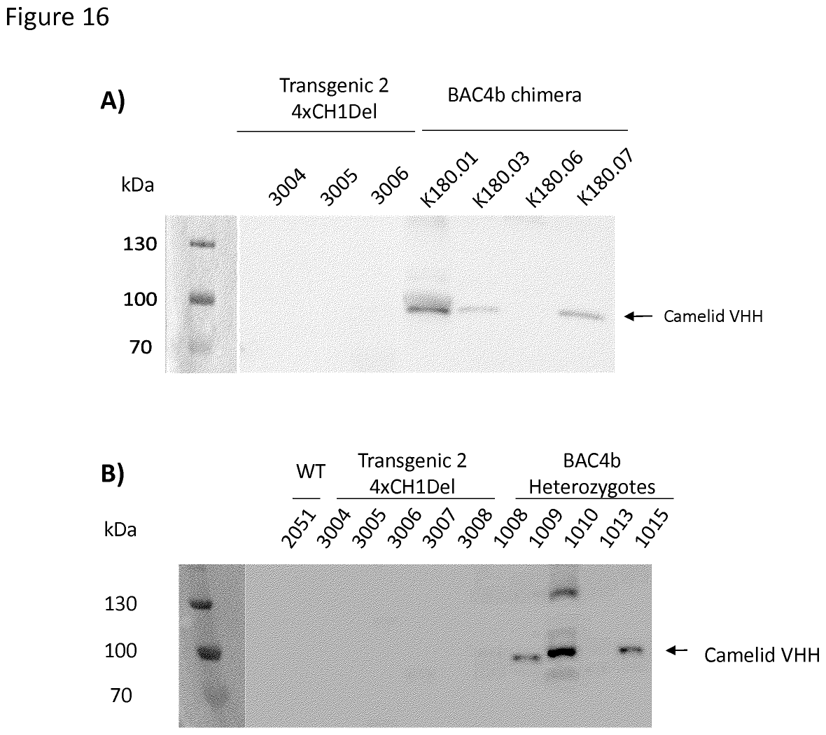

As illustrated in FIG. 16A, 3 out of 4 serum samples obtained from pre-immunized BAC4b chimeras showed camelid VHH expression compared to none from the Transgenic 2 animals with no camelid VHH insertion.

As illustrated in FIG. 16B, serum samples from pre-immunized F1 heterozygous litter of 5 animals derived from founder were used for Western blot detection. 4 out of 5 animals showed camelid VHH expression whereas none of Transgenic 2 animals or wild type control samples showed VHH expression.

These results show that BAC4b transgenic mouse can successfully use camelid VHH, D, and J genes from BAC4b insertion in VDJ recombination to express sdAbs and be detected in circulating blood.

Transgenic mice carrying camelid V, D and J segments are immunized with an antigen such as with Target 1, recombinant human CD3 ε and δ subunits, SARS-Cov-2 spike as described herein or with another antigen. Serum sample and spleen tissues are collected to construct libraries of variable heavy chains (VHHs). Two rounds of panning are performed against the antigen using phage display technology. DNA samples are collected and analyzed by NGS (Miseq, v600 cycle, 25 million reads). In parallel, 96 phage clones are picked from the second round of panning and tested for binding to recombinant protein by ELISA and binding to human PBMCs by flow cytometry. The nucleic acid of positive binders is obtained.

### Example 11—Increasing Diversity of Expressed Variable Regions and Single Domain Antibodies

To increase the diversity of antibodies from antigen immunizations, different homozygous transgenic animals are cross bred to generate heterologous animals carrying different IgH alleles with one or more different V, D and/or J segments. For example, homozygous animals carrying the BAC4b transgene are cross bred with homozygous animals carrying the BAC6 transgene resulting in heterozygous animals carrying both transgenes and thus increasing the diversity of sdAbs produced by immunization.

### Example 12—Generating Monospecific, Multispecific and Multivalent Binding Agents

As described herein, variable regions of selected binders may be cloned to incorporate constant regions, Fc, or into other format including without limitation those disclosed in Deyev, S. M et al. (BioEssays 30:904-918, 2008) and in PCT/CA2020/051753 published on Jun. 24, 2021 under number WO2021119832A1.

The binding agents thus created are tested for binding specificity, affinity and/or biological activity.

The invention is not limited to particular material, methods or experimental conditions described herein as such the material, methods or experimental conditions may vary. The embodiments and examples described herein are illustrative and are not meant to limit the scope of the disclosure as claimed. Those skilled in the art will recognize or be able to ascertain using no more than routine experimentation, many equivalents to the specific embodiments of the invention described herein. Variations of the embodiments, including alternatives, modifications combinations, permutations and equivalents, are intended to be encompassed by the present disclosure.

All documents, patents, journal articles and other material cited in the present application are hereby incorporated herein by reference.

### REFERENCES

The content of all patents, patent applications and publications referred to throughout the application are incorporated herein by reference.
Deyev, S. M et al. BioEssays 30:904-918 (2008).
Drabek et al. Front. Immunol. 7:619 (2016).
Janssens et al., PNAS 103(41):15130-15135 (2006).
Hamers-Casterman C, et al., Naturally-occurring Antibodies Devoid of Light-chains. Nature 1993, 363:446-448.
Muyldermans, S and Smider, 2016. Distinct Antibody Species: Structural Differences Creating Therapeutic Opportunities. Current Opinion in Immunology 2016, 40:7-13.
Muyldermans, S et al., 1994. Sequence and Structure of VH Domain From Naturally Occurring Camel Heavy Chain Immunoglobulins Lacking Light Chains. Protein Eng. 7: 1129-1135.
Sircar et al., The Journal of Immunology, 186, 2011.
U.S. Pat. No. 8,502,014 in the name of Grosveld.
US2011/0145937A1 in the name of Regeneron.
WO2016/062990A1 in the name of Crescendo Biologics Limited.
WO2021119832A1 in the name of KisoJi Biotechnology Inc.
Zhou et al. J. Immunol., 175(6):3369-79 (2005).

1. A transgenic non-human animal comprising germline modifications at an immunoglobulin heavy chain (IgH) locus, wherein the IgH locus comprises a) unrearranged heavy chain variable (V), diversity (D) and joining (J) gene segments and wherein the D and/or J gene segments comprise camelid D and/or J gene segments and b) at least one IgG constant region gene lacking a functional CH1 domain

2. The transgenic non-human animal of claim 1, wherein the transgenic non-human animal is capable of expressing heavy chain only antibodies (HCAbs) or nucleic acids encoding same.

3. The transgenic non-human animal of claim 1 or 2, wherein the modifications comprise a) replacement of one or more endogenous non-human D and/or J gene segments for one or more unrearranged camelid D and/or J gene segments and b) partial or complete deletion of the CH1 domain of at least one IgG constant region gene.

4. The transgenic non-human animal of claim 1 or 2, wherein the modifications comprise a) insertion of one or more unrearranged camelid D and/or J gene segments and b) partial or complete deletion of the CH1 domain of at least one IgG constant region gene.

5. The transgenic non-human animal of claim 1 or 2, wherein the modifications comprise a) replacement of one or more endogenous non-human D and/or J gene segments for one or more unrearranged camelid D and/or J gene segments or insertion of one or more unrearranged camelid D and/or J gene segments and b) modification of the CH1 domain of at least one IgG constant region gene.

6. The transgenic non-human animal of any of claims 1 to 5, wherein the modifications comprise replacement of all endogenous non-human D and J segments with unrearranged camelid D and J gene segments.

7. The transgenic non-human animal of any one of claims 1 to 6, wherein the camelid D and/or J gene segments are from a single camelid species.

8. The transgenic non-human animal of any one of claims 1 to 6, wherein the camelid D and/or J gene segments are from at least two, at least three or at least four camelid species.

9. The transgenic non-human animal of any of claims 1 to 8, wherein the modifications further comprise replacement of one or more endogenous non-human V gene segments with V gene segments of multiple mammal species or insertion of V gene segments of multiple mammal species.

10. The transgenic non-human animal of any of claims 1 to 8, wherein the modifications further comprise replacement of one or more endogenous non-human V gene segments with one or more camelid V gene segments or insertion of camelid V gene segments.

11. The transgenic non-human animal of claim 10, wherein the modifications comprise replacement of all endogenous non-human V segments for camelid V gene segments.

12. The transgenic non-human animal of any one of claims 1 to 11, wherein the V gene segments are from at least two species.

13. The transgenic non-human animal of any one of claims 1 to 11, wherein the V gene segments are from at least three species.

14. The transgenic non-human animal of any one of claims 1 to 11, wherein the V gene segments are from at least four species.

15. The transgenic non-human animal of any of the preceding claims, wherein the V gene segments encode a VH or VHH polypeptide.

16. The transgenic non-human animal of claim 15, wherein the VH or VHH polypeptide is a camelid VH or camelid VHH polypeptide.

17. The transgenic non-human animal of claim 16, wherein the camelid VH polypeptide is from an alpaca, a llama, a Bactrian, a Vicuna or a dromedary.

18. The transgenic non-human animal of claim 16, wherein the camelid VHH polypeptide is from an alpaca, a llama, a Bactrian, a Vicuna or a dromedary.

19. The transgenic non-human animal of any of the preceding claims, wherein the transgenic non-human animal comprises V segments from an alpaca, V segments from a Bactrian, V segments from a llama, V segments from a Vicuna and/or V segments from a dromedary.

20. The transgenic non-human animal of any of the preceding claims, wherein the camelid D and/or J gene segments are from an alpaca.

21. The transgenic non-human animal of any of the preceding claims, wherein the camelid D and/or J gene segments are from a Bactrian.

22. The transgenic non-human animal of any of the preceding claims, wherein the camelid D and/or J gene segments are from a llama.

23. The transgenic non-human animal of any of the preceding claims, wherein the camelid D and/or J gene segments are from a dromedary.

24. The transgenic non-human animal of any of the preceding claims, wherein the camelid D and/or J gene segments are from a Vicuna.

25. The transgenic non-human animal of any of the preceding claims, wherein the IgH locus comprises from one to at least seven D gene segments of alpacas.

26. The transgenic non-human animal of any of the preceding claims, wherein the IgH locus comprises from one to at least seven J gene segments of alpacas.

27. The transgenic non-human animal of any of the preceding claims, wherein the IgH locus comprises from one to at least seven Bactrian D gene segments.

28. The transgenic non-human animal of any of the preceding claims, wherein the IgH locus comprises from one to at least seven Bactrian J gene segments.

29. The transgenic non-human animal of any of the preceding claims, wherein the transgenic non-human animal comprises from one to at least six V gene segment of alpacas.

30. The transgenic non-human animal of any of the preceding claims, wherein the transgenic non-human animal comprises from one to at least ten V gene segment of Bactrians.

31. The transgenic non-human animal of any of the preceding claims, wherein the transgenic non-human animal comprises from one to at least ten V gene segment of llamas.

32. The transgenic non-human animal of any of the preceding claims, wherein the transgenic non-human animal comprises from one to at least six V gene segment of dromedaries.

33. The transgenic non-human animal of any of the preceding claims, wherein the transgenic non-human animal comprises from one to at least six V gene segment of Vicunias.

34. The transgenic non-human animal of any of the preceding claims, wherein the V gene segments, D gene segments and/or J gene segments encode a naturally occurring sequence.

35. The transgenic non-human animal of any of the preceding claims, wherein the V gene segments, D gene segments and/or J gene segments encode a mutated sequence.

36. The transgenic non-human animal of any of the preceding claims, wherein the IgG constant region gene is an endogenous non-human IgG constant region gene or a portion thereof.

37. The transgenic non-human animal of any of the preceding claims, wherein the IgG constant region gene is a γ3 constant region gene, a γ1 constant region gene, a γ2b constant region gene or a γ2a constant region gene.

38. The transgenic non-human animal of any of the preceding claims, wherein at least two IgG constant region genes comprise a partial or complete deletion in the region encoding the CH1 domain.

39. The transgenic non-human animal of any of the preceding claims, wherein at least three IgG constant region genes comprise a partial or complete deletion in the region encoding the CH1 domain.

40. The transgenic non-human animal of any of the preceding claims, wherein all IgG constant region genes comprise a partial or complete deletion in the region encoding the CH1 domain.

41. The transgenic non-human animal of any of the preceding claims, wherein at least one IgG constant region gene comprises a partial or complete deletion in the region encoding the CH1 domain and at least one other IgG constant region gene is completely or partially deleted.

42. The transgenic non-human animal of any of the preceding claims, wherein the IgH locus comprises a γ3 constant region gene comprising a partial or complete deletion in the region encoding the CH1 domain at one or both alleles.

43. The transgenic non-human animal of any of the preceding claims, wherein the IgH locus comprises a γ1 constant region gene comprising a partial or complete deletion in the region encoding the CH1 domain at one or both alleles.

44. The transgenic non-human animal of any of the preceding claims, wherein the IgH locus comprises a γ2b constant region gene comprising a partial or complete deletion in the region encoding the CH1 domain at one or both alleles.

45. The transgenic non-human animal of any of the preceding claims, wherein the IgH locus comprises a γ2a constant region gene comprising a partial or complete deletion in the region encoding the CH1 domain at one or both alleles.

46. The transgenic non-human animal of any one of the preceding claims, wherein at least one allele of the transgenic non-human animal genome comprises an IgH locus comprising an IgG constant region gene selected from the group consisting of a γ3 constant region gene comprising a partial or complete deletion in the region encoding the CH1 domain, a yl constant region gene comprising a partial or complete deletion in the region encoding the CH1 domain, a γ2b constant region gene comprising a partial or complete deletion in the region encoding the CH1 domain, a γ2a constant region gene comprising a partial or complete deletion in the region encoding the CH1 domain or combination thereof.

47. The transgenic non-human animal of any one of the preceding claims, wherein one allele of the transgenic non-human animal genome comprises an IgH locus comprising a partial or complete deletion of the γ3 and γ2b constant region genes and γ1 and γ2a constant region genes comprising a partial or complete deletion in the region encoding the CH1 domain.

48. The transgenic non-human animal of any the preceding claims, wherein one allele of the transgenic non-human animal genome comprises an IgH locus comprising a γ2b constant region gene comprising a partial or complete deletion in the region encoding the CH1 domain.

49. The transgenic non-human animal of any of the preceding claims, wherein one allele of the transgenic non-human animal genome comprises an IgH locus comprising a γ3 constant region gene comprising a partial or complete deletion in the region encoding the CH1 domain.

50. The transgenic non-human animal of any of the preceding claims, wherein one allele of the transgenic non-human animal genome comprises an IgH locus comprising a γ3 constant region gene comprising a partial or complete deletion in the region encoding the CH1 domain and a γ2a constant region gene comprising a partial or complete deletion in the region encoding the CH1 domain.

51. The transgenic non-human animal of any of the preceding claims, wherein one allele of the transgenic non-human animal genome comprises an IgH locus comprising a γ3 constant region gene comprising a partial or complete deletion in the region encoding the CH1 domain, a γ2a constant region gene comprising a partial or complete deletion in the region encoding the CH1 domain and a partial or complete deletion of the γ2b constant region gene.

52. The transgenic non-human animal of any of the preceding claims, wherein one allele of the transgenic non-human animal genome comprises an IgH locus comprising a γ3 constant region gene comprising a partial or complete deletion in the region encoding the CH1 domain and a γ2b constant region gene comprising a partial or complete deletion in the region encoding the CH1 domain.

53. The transgenic non-human animal of any one of claims 46 to 52, wherein the other allele of the transgenic non-human animal genome comprises an identical IgH locus or an identical IgG constant region gene.

54. The transgenic non-human animal of any one of claims 46 to 52, wherein the other allele of the transgenic non-human animal genome comprises a wild type IgH locus or a wild type an IgG constant region gene.

55. The transgenic non-human animal of any one of claims 46 to 52, wherein the other allele of the transgenic non-human animal genome comprises an IgH locus comprising a modification selected from a partial or complete deletion in the region encoding the CH1 domain of at least one or all IgG constant region genes, a complete or partial deletion of at least one or all other IgG constant region genes or a combination thereof.

56. The transgenic non-human animal of any one of claims 46 to 52, wherein the other allele comprises an IgH locus comprising wild type non-human γ3, γ1, γ2b and γ2a constant region genes.

57. The transgenic non-human animal of any one of claims 46 to 52, wherein the other allele comprises an IgH locus comprising a partial or complete deletion of the γ3, γ1 and γ2b constant region genes and a γ2a constant region gene comprising a partial or complete deletion in the region encoding the CH1 domain.

58. The transgenic non-human animal of any one of claims 46 to 52, wherein the other allele comprises an IgH locus comprising γ3 and γ2a constant region genes comprising a partial or complete deletion in the region encoding the CH1 domain and a partial or complete deletion of the γ2b constant region gene.

59. The transgenic non-human animal of any one of claims 46 to 52, wherein the other allele comprises an IgH locus comprising a γ3 constant region gene comprising a partial or complete deletion in the region encoding the CH1 domain.

60. The transgenic non-human animal of any one of claims 46 to 52, wherein the other allele comprises an IgH locus comprising a partial or complete deletion of the γ3, γ1 and γ2b constant region genes.

61. The transgenic non-human animal of any one of claims 46 to 52, wherein the other allele comprises an IgH locus comprising γ3 and γ2a constant region genes comprising a partial or complete deletion in the region encoding the CH1 domain and a partial or complete deletion of γ2b constant region gene.

62. The transgenic non-human animal of any one of claims 1 to 37, wherein both alleles of the transgenic non-human animal genome comprise an IgH locus comprising a γ2b constant region gene comprising a partial or complete deletion in the region encoding the CH1 domain.

63. The transgenic non-human animal of any one of claims 1 to 37, wherein both alleles of the transgenic non-human animal genome comprise an IgH locus comprising γ3 and γ2a constant region genes comprising a partial or complete deletion in the region encoding the CH1 domain and a partial or complete deletion of γ2b constant region gene.

64. The transgenic non-human animal of any one of claims 1 to 37, wherein both alleles of the transgenic non-human animal genome comprise an IgH locus comprising γ3, γ1, γ2b and γ2a constant region genes comprising a partial or complete deletion in the region encoding the CH1 domain.

65. The transgenic non-human animal of any one of claims 1 to 37, wherein both alleles of the transgenic non-human animal genome comprise an IgH locus comprising γ3, γ1, γ2b and γ2a constant region genes comprising a complete deletion in the region encoding the CH1 domain.

66. The transgenic non-human animal of any of the preceding claims, wherein the non-human animal genome comprises at least one different V gene segment on each allele.

67. The transgenic non-human animal of any of the preceding claims, wherein the non-human animal genome comprises at least one V gene segment of one species in one of its alleles and at least one V gene segment of another species in the other allele.

68. The transgenic non-human animal of any of the preceding claims, wherein the transgenic non-human animal further comprises a germline modification of an IgM constant region gene, wherein the modification comprises replacement of the IgM CH1 domain for a camelid CH1 domain.

69. The transgenic non-human animal of any of the preceding claims wherein the non-human animal comprises at least about 10 kb, at least about 20 kb, at least about 30 kb, at least about 40 kb or at least about 50 kb of camelid V gene segments of llama, Bactrian and/or alpaca species.

70. The transgenic non-human animal of any of the preceding claims, wherein the V gene segments, D gene segments and J gene segments are capable of VDJ rearrangement.

71. A transgenic non-human animal comprising germline modifications at an immunoglobulin heavy chain (IgH) locus, wherein the modification is selected from the group consisting of:
a. deletion of the CH1 domain of an endogenous non-human animal γ3 gene, γ1 gene, γ2b gene and/or or γ2a gene, or;
b. deletion of the CH1 domain of at least one endogenous non-human animal gene selected from γ3 gene, γ1 gene, γ2b gene and/or or γ2a gene in combination with a complete or partial deletion of at least one endogenous non-human animal gene selected from γ3 gene, γ1 gene, γ2b gene and/or or γ2a gene.

72. A transgenic non-human animal comprising germline modifications at an immunoglobulin heavy chain (IgH) locus, wherein the modification is selected from the group consisting of:
a. modification of the CH1 domain of an endogenous non-human animal γ3 gene, yl gene, γ2b gene and/or or γ2a gene, or;
b. modification of the CH1 domain of at least one endogenous non-human animal gene selected from γ3 gene, γ1 gene, γ2b gene and/or or γ2a gene in combination with a complete or partial deletion of at least one endogenous non-human animal gene selected from γ3 gene, γ1 gene, γ2b gene and/or or γ2a gene.

73. The transgenic non-human animal of claim 71 or 72, wherein the transgenic non-human animal comprises a V, D and/or J segments selected from mouse V, D and/or J, camelid V, D and/or J or human V, D and/or J or combination thereof.

74. The transgenic non-human animal of any of the preceding claims, wherein the transgenic non-human animal is heterozygous.

75. The transgenic non-human animal of any of the preceding claims, wherein the transgenic non-human animal is homozygous.

76. The transgenic non-human animal of any of the preceding claims, wherein the transgenic non-human animal is a transgenic rat.

77. The transgenic non-human animal of any of the preceding claims, wherein the transgenic non-human animal is a transgenic mouse.

78. The transgenic non-human animal of any of the preceding claims, wherein the non-human animal is a transgenic mouse comprising an IgG constant region gene encoding a mouse IgG1, a mouse IgG2a, a mouse IgG2b or a mouse IgG3 constant region lacking a CH1 domain.

79. The transgenic non-human animal of claim 78, wherein at least two IgG constant region genes selected from the mouse IgG1, a mouse IgG2a, a mouse IgG2b or a mouse IgG3 constant region lack a CH1 domain.

80. The transgenic non-human animal of claim 78, wherein at least three IgG constant region genes selected from the mouse IgG1, a mouse IgG2a, a mouse IgG2b or a mouse IgG3 constant region lack a CH1 domain.

81. The transgenic non-human animal of claim 78, wherein each of the mouse IgG1, mouse IgG2a, mouse IgG2b and mouse IgG3 constant region lack a CH1 domain.

82. The transgenic non-human animal of any one of claims 78 to 81, wherein at least one of the mouse IgG1, a mouse IgG2a, a mouse IgG2b or a mouse IgG3 is partially or completely deleted.

83. The transgenic non-human animal of any of the preceding claims, wherein the transgenic non-human animal is a transgenic mouse and wherein all endogenous mouse D and J gene segments are replaced with unrearranged camelid D and J gene segments.

84. The transgenic non-human animal of any of the preceding claims, wherein the transgenic non-human animal is a transgenic mouse and wherein the IgH locus of the transgenic mouse comprises one or more mouse V gene segments and unrearranged camelid V gene segments.

85. The transgenic non-human animal of any of the preceding claims, wherein the transgenic non-human animal is a transgenic mouse and wherein all endogenous mouse V gene segments are replaced with unrearranged camelid V gene segments.

86. The transgenic non-human animal of any of the preceding claims, wherein the transgenic non-human animal is a transgenic mouse having at least one endogenous mouse IgG constant region gene lacking a functional CH1 domain.

87. The transgenic non-human animal of any of the preceding claims, wherein the transgenic non-human animal is a transgenic mouse having all endogenous mouse IgG constant region genes of one allele lacking a functional CH1 domain.

88. The transgenic non-human animal of any of the preceding claims, wherein the transgenic non-human animal is a transgenic mouse having all endogenous mouse IgG constant region genes of both alleles lacking a functional CH1 domain.

89. The transgenic non-human animal of any one of claims 78 to 88, wherein the transgenic mouse is heterozygous and wherein one allele of the mouse genome comprises a partial or complete deletion in the region encoding the CH1 domain of at least one IgG constant region genes and optionally a complete or partial deletion of at least one other IgG constant region genes and the other allele is wild type.

90. The transgenic non-human animal of any one of claims 78 to 88, wherein the transgenic mouse is heterozygous and one allele of the mouse genome comprises a partial or complete deletion in the region encoding the CH1 domain of at least one IgG constant region genes and optionally a complete or partial deletion of at least one or all other IgG constant region genes and the other allele optionally comprises a partial or complete deletion in the region encoding the CH1 domain of at least one IgG constant region genes or a complete or partial deletion of at least one or all other IgG constant region genes or a combination thereof.

91. The transgenic non-human animal of any of the preceding claims, wherein the modification comprises deletion of the CH1 domain of the endogenous γ3 gene.

92. The transgenic non-human animal of any of the preceding claims, wherein the modification comprises deletion of the CH1 domain of the endogenous γ2b gene.

93. The transgenic non-human animal of any of the preceding claims, wherein the modification comprises deletion of the CH1 domain of each of the endogenous γ3 gene, γ1 gene, γ2b gene and γ2a gene.

94. The transgenic non-human animal of any of the preceding claims, wherein the modification comprises deletion of the CH1 domain of the endogenous γ2a gene and deletion of the endogenous γ2b gene.

95. The transgenic non-human animal of any of the preceding claims, wherein the modification comprises deletion of the CH1 domain of each of the endogenous γ3 gene and γ2a gene and deletion of the γ2b gene.

96. The transgenic non-human animal of any one of claims 78 to 95, wherein the transgenic mouse is homozygous.

97. The transgenic non-human animal of any one of the preceding claims, wherein the transgenic non-human animal is a transgenic mouse and wherein the germline modifications at the IgH locus comprise a) replacement of the endogenous mouse D and J gene segments for unrearranged camelid D and J gene segments a) replacement of one or more of the endogenous mouse V gene segments for one or more unrearranged camelid V gene segments or insertion of one or more unrearranged camelid V gene segments and c) deletion or modification of the CH1 domain of at least one or all of endogenous mouse γ1, γ2a, γ2b or γ3 gene so that a polypeptide expressed from said endogenous mouse γ1, γ2a, γ2b or γ3 gene does not comprise a functional CH1 domain.

98. The transgenic non-human animal of any one of the preceding claims, wherein the transgenic non-human animal is a transgenic mouse comprising germline modifications at an immunoglobulin heavy chain (IgH) locus, wherein the modification comprises deletion of the CH1 domain of each of the endogenous mouse γ3 gene, γ1 gene, γ2b gene and γ2a gene, replacement of endogenous mouse D and J gene segments for unrearranged camelid D and J gene segments, insertion of camelid V gene segments from multiple camelid species and optionally deletion of at least one or all endogenous mouse V gene segments.

99. The transgenic non-human animal of any one of the preceding claims, wherein the unrearranged camelid V gene segments include associated introns comprising recombination signal sequences for VDJ rearrangement.

100. The transgenic non-human animal of any one of the preceding claims, wherein the camelid V segments encodes VH and VHH polypeptides.

101. The transgenic non-human animal of any one of the preceding claims, wherein the transgenic non-human animal is capable of expressing heavy chain only antibodies (HCAbs) or nucleic acids encoding same following immunization with an antigen.

102. The transgenic non-human animal of any one of the preceding claims, wherein the transgenic non-human animal is a transgenic mouse capable of expressing heavy chain only antibodies (HCAbs) comprising a mouse VH polypeptide comprising camelid canonical framework mutations at position 37, 44, 45 and/or 47.

103. The transgenic non-human animal of any one of the preceding claims, wherein the camelid V segments encodes VH and/or VHH polypeptides from an alpaca, a Bactrian and a llama.

104. The transgenic non-human animal of any one of the preceding claims, wherein the camelid V segments encodes VH and/or VHH polypeptides from an alpaca, a Bactrian, a llama and a dromedary.

105. The transgenic non-human animal of any one of claims 78 to 104, wherein the transgenic mouse has an MHC haplotype characterized as H-2^{b}.

106. A transgenic mouse comprising endogenous mouse V, D and J segments and at least one endogenous mouse IgG constant region gene lacking a functional CH1 domain, wherein the transgenic mouse is capable of expressing heavy chain only antibodies (HCAbs).

107. The transgenic mouse of claim 106, wherein the transgenic mouse does not comprise foreign V, D or J segments.

108. The transgenic mouse of claim 106, wherein the transgenic mouse comprises camelid V, D and/or J segments.

109. The transgenic mouse of any one of claims 106 to 109, wherein the transgenic mouse is capable of expressing heavy chain only antibodies (HCAbs) comprising a mouse VH polypeptide comprising camelid canonical framework mutations at position 37, 44, 45 and/or 47.

110. A method for obtaining antigen-specific heavy chain only antibodies (HCAbs) or nucleic acids encoding an antigen-binding domain of the HCAbs or a portion thereof, the method comprising immunizing the transgenic non-human animal of any one of the preceding claims with an antigen.

111. The method of claim 110, wherein the transgenic non-human animal produces a plurality of HCAbs upon immunization with the antigen and wherein the plurality of HCAbs comprises at least one HCAb species comprising a V portion encoded by a V segment of a first mammal species and a second HCAb species comprising V portion encoded by a V segment of a second mammal species.

112. The method of claim 110 or 111, wherein the method further comprises collecting total RNA or messenger RNAs from the transgenic non-human animal's PBMCs.

113. The method of any of the preceding claims further comprising determining the amino acid sequence or nucleic acid sequence of one or more complementarity determining regions or variable region of the HCAb species.

114. The method of claim 113, further comprising using a computer-based method or software for organizing the sequence information in clusters based on predetermined parameters.

115. The method of claim 114, further comprising selecting one or more sequences to make a binding agent.

116. The method of claim 115, wherein the binding agent is an antibody or an antigen binding fragment thereof.

117. The method of claim 115, wherein the binding agent comprises a VHH.

118. The method of claim 115, wherein the binding agent comprises a single domain antibody.

119. The method of any of the preceding claims, wherein the transgenic non-human animal is a transgenic mouse comprising germline modifications at an IgH locus comprising a) replacement of one or more of the endogenous mouse V gene segments for one or more unrearranged camelid V gene segments or insertion of unrearranged camelid V gene segments, b) replacement of at least one or all of the endogenous mouse D and J segments with camelid D and J segments and c) deletion or modification of the CH1 domain of at least one or all of endogenous mouse γ1, γ2a, γ2b and γ3 gene so that a polypeptide expressed from said endogenous mouse γ1, γ2a, γ2b and γ3 gene does not comprise a functional CH1 domain.

120. The method of any of the preceding claims, wherein the transgenic non-human animal is a transgenic mouse comprising germline modifications at an immunoglobulin heavy chain (IgH) locus, wherein the modification comprises deletion of the CH1 domain of each of the endogenous mouse γ3 gene, γ1 gene, γ2b gene and γ2a gene, replacement of mouse D and J gene segments for unrearranged camelid D and J gene segments, insertion of camelid V gene segments from multiple camelid species and optionally deletion of at least one or all endogenous mouse V gene segments.

121. A method for making a binding agent, the method comprising immunizing the transgenic non-human animal of any of the preceding claims with an antigen, obtaining the amino acid sequence or nucleic acid sequence of an antigen-binding domain of at least one HCAb species and generating a binding agent comprising the amino acid sequence.

122. The method of claim 121, wherein the antigen-binding domain comprises one or more complementarity determining regions or variable region of at least one HCAb species.

123. The method of claim 121 or 122, wherein the amino acid sequence or nucleic acid sequence of one or more complementarity determining regions or variable region of a plurality of HCAb species is obtained and a binding agent comprising a most represented or a common sequence is generated.

124. The method of claim 121 or 122, wherein the amino acid sequence or nucleic acid sequence of one or more complementarity determining regions or variable region of a plurality of HCAb species is obtained and a binding agent comprising a least represented or a unique sequence is generated.

125. A binding agent comprising an amino acid sequence or encoded by a nucleic acid sequence obtained by the method of any one of claims 121 to 124.

126. A binding agent comprising an amino acid sequence or encoded by a nucleic acid sequence obtained by immunizing the transgenic non-human animal of any one of claims 1-105.

127. A nucleic acid construct for targeted replacement of non-human animal genomic D and/or J segments or insertion of camelid D and/or J segments at an IgH locus, wherein the nucleic acid construct comprises genomic camelid D and/or J segments and optionally comprises genomic camelid V segments and wherein the nucleic acid construct comprises introns comprising recombination signal sequences for VDJ rearrangement.

128. The nucleic acid construct of claim 127, wherein the nucleic acid construct comprises genomic camelid V segments from at least one species.

129. The nucleic acid construct of claim 127, wherein the nucleic acid construct comprises camelid V segments from at least two species.

130. The nucleic acid construct of claim 127, wherein the nucleic acid construct comprises camelid V segments from at least three species.

131. The nucleic acid construct of claim 127, wherein the nucleic acid construct comprises camelid V segments from at least four species.

132. The nucleic acid construct of any one of claims 127 to 131, wherein the nucleic acid construct comprises D and J segments from at least one camelid species.

133. The nucleic acid construct of any one of claims 127 to 131, wherein the nucleic acid construct comprises D and J segments from at least two camelid species.

134. The nucleic acid construct of any one of claims 127 to 133, wherein the nucleic acid construct comprises from one to at least seven alpaca D gene segments.

135. The nucleic acid construct of any one of claims 127 to 134, wherein the nucleic acid construct comprises from one to at least seven alpaca J gene segments.

136. The nucleic acid construct of any one of claims 127 to 135, wherein the nucleic acid construct comprises from one to at least seven Bactrian D gene segments.

137. The nucleic acid construct of any one of claims 127 to 136, wherein the nucleic acid construct comprises from one to at least seven Bactrian J gene segments.

138. The nucleic acid construct of any one of claims 127 to 137, wherein the nucleic acid construct comprises from one to at least six alpaca V gene segments.

139. The nucleic acid construct of any one of claims 127 to 138, wherein the nucleic acid construct comprises from one to at least ten Bactrians V gene segments.

140. The nucleic acid construct of any one of claims 127 to 139, wherein the nucleic acid construct comprises from one to at least ten llama V gene segments.

141. The nucleic acid construct of any one of claims 127 to 140, wherein the nucleic acid construct comprises from one to at least six dromedaries V gene segments.

142. The nucleic acid construct of any of the preceding claims, wherein the nucleic acid comprises in a 5′ to 3′ fashion, alpaca VH and/or VHH segments, alpaca D segments and alpaca J segments.

143. The nucleic acid construct of any of the preceding claims, wherein the nucleic acid comprises in a 5′ to 3′ fashion, Bactrian VH and/or VHH segments, alpaca VH and/or VHH segments, alpaca D segments and alpaca J segments.

144. The nucleic acid construct of any of the preceding claims, wherein the nucleic acid comprises in a 5′ to 3′ fashion, llama VH and/or VHH segments, alpaca VH and/or VHH segments, alpaca D segments and alpaca J segments.

145. The nucleic acid construct of any of the preceding claims, wherein the nucleic acid comprises in a 5′ to 3′ fashion, llama VH and/or VHH segments, Bactrian VH and/or VHH segments, alpaca VH and/or VHH segments, alpaca D segments and alpaca J segments.

146. The nucleic acid construct of any of the preceding claims, wherein the nucleic acid comprises in a 5′ to 3′ fashion, Bactrian VH and/or VHH segments, llama VH and/or VHH segments, alpaca VH and/or VHH segments, alpaca D segments and alpaca J segments.

147. The nucleic acid construct of any of the preceding claims, wherein the nucleic acid comprises in a 5′ to 3′ fashion, llama VH and/or VHH segments, Bactrian VH and/or VHH segments, alpaca VH and/or VHH segments, alpaca D segments, Bactrian D segments, Bactrian J segments, and alpaca J segments.

148. The nucleic acid construct of any of the preceding claims, wherein the nucleic acid comprises in a 5′ to 3′ fashion, llama VH and/or VHH segments, Bactrian VH and/or VHH segments, alpaca VH and/or VHH segments, Bactrian D segments and Bactrian J segments.

149. The nucleic acid construct of any of the preceding claims, wherein the nucleic acid comprises in a 5′ to 3′ fashion, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

150. The nucleic acid construct of any of the preceding claims, wherein the nucleic acid comprises in a 5′ to 3′ fashion, alpaca VH and/or VHH segments, Bactrian VH and/or VHH segments, alpaca VH and/or VHH segments, llama VH and/or VHH segments, dromedary VH and/or VHH segments, llama VH and/or VHH segments, Bactrian VH and/or VHH segments, alpaca VH and/or VHH segments, alpaca D segments and alpaca J segments.

151. The nucleic acid construct of any one of claims 127 to 150, wherein the nucleic acid construct comprises mouse VH segments at a 5′ end or 3′ end.

152. The nucleic acid construct of any one of claims 127 to 150, wherein the nucleic acid construct does not comprise mouse VH segments at a 5′ or 3′ end.

153. The nucleic acid construct of any one of claims 127 to 151, wherein the nucleic acid construct is an artificial chromosome.

154. Use of the nucleic acid construct of any one of claims 127 to 152 for modifying embryonic non-human stem cells or for making a transgenic non-human animal.

155. Isolated embryonic non-human stem cells modified by the nucleic acid construct of any one of claims 127 to 154.

156. A cell isolated from the transgenic non-human animal of any one of the preceding claims.

157. Isolated embryonic non-human stem cells comprising germline modifications at an immunoglobulin heavy chain (IgH) locus, wherein the IgH locus comprises a) unrearranged heavy chain variable (V), diversity (D) and joining (J) gene segments and wherein the D and/or J gene segments comprise camelid D and/or J gene segments and b) at least one IgG constant region gene lacking a functional CH1 domain.

158. The isolated embryonic non-human stem cell of claim 157, wherein isolated embryonic non-human stem cell is a mouse embryonic stem cell and wherein the modification comprises a) replacement of one or more of the endogenous mouse V gene segments for one or more unrearranged camelid V gene segments or insertion of unrearranged camelid V gene segments, b) replacement of at least one or all of the endogenous mouse D and J segments with camelid D and J segments and c) deletion or modification of the CH1 domain of at least one or all of endogenous mouse γ1, γ2a, γ2b and γ3 gene so that a polypeptide expressed from said endogenous mouse γ1, γ2a, γ2b and γ3 gene does not comprise a functional CH1 domain.

159. The isolated embryonic non-human stem cell of claim 157, wherein isolated embryonic non-human stem cell is a mouse embryonic stem cell and wherein the modification comprises deletion of the CH1 domain of each of the endogenous γ3 gene, γ1 gene, γ2b gene and γ2a gene, replacement of endogenous mouse D and J gene segments for unrearranged camelid D and J gene segments, insertion of camelid V gene segments from multiple camelid species and optionally deletion of at least one or all endogenous mouse V gene segments.

160. Use of the embryonic non-human stem cells of claim 155 or 157-159, in the making of a transgenic non-human animal.

161. A process of producing a transgenic non-human animal, the process comprising the step of injecting the embryonic non-human stem cells of claim 155 or 157-159 into a mouse blastocyst, implanting the mouse embryo into a pseudopregnant mouse and selecting the mouse progeny carrying the germline modifications.

162. A method of making a transgenic animal comprising use of the nucleic acid construct of any one of claims 127 to 153.

163. A method of making a transgenic animal comprising introducing a nucleic acid construct into a stem cell, the nucleic acid comprising a genomic camelid D and/or J segments and optionally comprises genomic camelid V segments and wherein the nucleic acid construct comprises introns comprising recombination signal sequences for VDJ rearrangement.

164. The method of claim 163, wherein the nucleic acid comprises V, D and/or J genetic sequences from at least two, three or four distinct species.

165. The method of claim 163, wherein the nucleic acid comprises V, D and/or J genetic sequences from at least two, three or four camelid species.

166. A method of making a transgenic mouse comprising implanting a blastocyst microinjected with embryonic stem cells genetically modified with the nucleic acid construct of any one of claims 127 to 153 into a pseudopregnant mouse, selecting chimeric mice from litter and optionally generating F1 heterozygous animals by backcrossing a chimeric mouse with a wild type mouse and optionally generating F2 homozygous animals by crossing F1 animals.

167. A method of making a transgenic mouse comprising implanting a blastocyst microinjected with the embryonic stem cells of any one of claim 154 or 157-159, selecting chimeric mice from litter and optionally generating F1 heterozygous animals by backcrossing a chimeric mouse with a wild type animal and optionally generating F2 homozygous animals by crossing F1 animals.
